SEQUENCE LISTING (281 sequences)

SEQ ID NO: 1 - DNA, 25 bp, Artificial Sequence
  synthetic
ttaggtgtatgggaggaagtacgga

SEQ ID NO: 2 - DNA, 25 bp, Artificial Sequence
  synthetic
aaaaaaaacgcgatctccaacgaaa

SEQ ID NO: 3 - DNA, 25 bp, Artificial Sequence
  synthetic
cgtcgttttgttggttatttgcgtg

SEQ ID NO: 4 - DNA, 23 bp, Artificial Sequence
  synthetic
accgaacgcgacccctttattcg

SEQ ID NO: 5 - DNA, 25 bp, Artificial Sequence
  synthetic
tatggattgtacggtagtcgggcgg

SEQ ID NO: 6 - DNA, 32 bp, Artificial Sequence
  synthetic
acgcaaacctcttaaccttctctcacaaatcg

SEQ ID NO: 7 - DNA, 24 bp, Artificial Sequence
  synthetic
ggcgttggtttgcggaaaggttac

SEQ ID NO: 8 - DNA, 25 bp, Artificial Sequence
  synthetic
cacccaccaaaactcgaaatacgct

SEQ ID NO: 9 - DNA, 25 bp, Artificial Sequence
  synthetic
tcgtcgtcgttattattaatacgtt

SEQ ID NO: 10 - DNA, 20 bp, Artificial Sequence
  synthetic
ataacctaccccgcccacgt

SEQ ID NO: 11 - DNA, 25 bp, Artificial Sequence
  synthetic
gagtttggtagtgggaggagatcgt

SEQ ID NO: 12 - DNA, 25 bp, Artificial Sequence
  synthetic
ccgaaacgaactaaaactactcgat

SEQ ID NO: 13 - DNA, 28 bp, Artificial Sequence
  synthetic
ggggcgatttttagtaagtttttttcgt

SEQ ID NO: 14 - DNA, 27 bp, Artificial Sequence
  synthetic
ctaccctctctatcctaacccctcgac

SEQ ID NO: 15 - DNA, 25 bp, Artificial Sequence
  synthetic
tcgggatttttttaggggtaatcga

SEQ ID NO: 16 - DNA, 27 bp, Artificial Sequence
  synthetic
gaaaaaacgaccgcaaaaattcacgta

SEQ ID NO: 17 - DNA, 25 bp, Artificial Sequence
  synthetic
ttgtatttttgtttgcgacgggggc

SEQ ID NO: 18 - DNA, 25 bp, Artificial Sequence
  synthetic
ccgaaaatcgataaaaacccgcgaa

SEQ ID NO: 19 - DNA, 25 bp, Artificial Sequence
  synthetic
ttttttttagttgttcgttcgtcgg

SEQ ID NO: 20 - DNA, 23 bp, Artificial Sequence
  synthetic
gacactccttaaaaccacgcgtt

SEQ ID NO: 21 - DNA, 23 bp, Artificial Sequence
  synthetic
gggtttataagttcggaggtcga

SEQ ID NO: 22 - DNA, 20 bp, Artificial Sequence
  synthetic
cacccaacacctaacgacga

SEQ ID NO: 23 - DNA, 25 bp, Artificial Sequence
  synthetic
gggcgaggttagaggggttgttttc

SEQ ID NO: 24 - DNA, 26 bp, Artificial Sequence
  synthetic
gcctcctactaaaactccaacccccg

SEQ ID NO: 25 - DNA, 28 bp, Artificial Sequence
  synthetic
acgtcgcgtattgtaaatatttttcgtg

SEQ ID NO: 26 - DNA, 28 bp, Artificial Sequence
  synthetic
cttcctaatacgaatcaacgaatcgtcc

SEQ ID NO: 27 - DNA, 26 bp, Artificial Sequence
  synthetic
cgaaagatcgatcgattgttcgacgg

SEQ ID NO: 28 - DNA, 21 bp, Artificial Sequence
  synthetic
ttataacgacgaattccgaaa

SEQ ID NO: 29 - DNA, 22 bp, Artificial Sequence
  synthetic
cggtcggaatttcggttttcgc

SEQ ID NO: 30 - DNA, 26 bp, Artificial Sequence
  synthetic
aacactaaccgcgcctaatatcgcta

SEQ ID NO: 31 - DNA, 26 bp, Artificial Sequence
  synthetic
cgggatagaggcgagagttcgaattc

SEQ ID NO: 32 - DNA, 32 bp, Artificial Sequence
  synthetic
gaatatttccaaacaaaaaccccaaatcctcg

SEQ ID NO: 33 - DNA, 20 bp, Artificial Sequence
  synthetic
cggttttgattttagggcgt

SEQ ID NO: 34 - DNA, 27 bp, Artificial Sequence
  synthetic
ttaactatcgaaaacgatttccgcgaa

SEQ ID NO: 35 - DNA, 25 bp, Artificial Sequence
  synthetic
tttttcgagtcgttttatttcgcgg

SEQ ID NO: 36 - DNA, 25 bp, Artificial Sequence
  synthetic
gaactccgaacgccgcttaaacgta

SEQ ID NO: 37 - DNA, 25 bp, Artificial Sequence
  synthetic
atttttgattatgggttttggtcgg

SEQ ID NO: 38 - DNA, 21 bp, Artificial Sequence
  synthetic
gaattcacctcgcccctcgct

SEQ ID NO: 39 - DNA, 32 bp, Artificial Sequence
  synthetic
taaataaaatattggtttgtagacggacgcgg

SEQ ID NO: 40 - DNA, 25 bp, Artificial Sequence
  synthetic
cgaactaacgctaaactcgcgcgat

SEQ ID NO: 41 - DNA, 25 bp, Artificial Sequence
  synthetic
ggggcgatagtatttaggtcgtcga

SEQ ID NO: 42 - DNA, 25 bp, Artificial Sequence
  synthetic
tcaccccaactaaaaaaactccgaa

SEQ ID NO: 43 - DNA, 25 bp, Artificial Sequence
  synthetic
tcgtagaggttatcggatatagcga

SEQ ID NO: 44 - DNA, 24 bp, Artificial Sequence
  synthetic
aatacccgaaccacaaaacccgcc

SEQ ID NO: 45 - DNA, 25 bp, Artificial Sequence
  synthetic
ttgcggaggcgacggagatattatc

SEQ ID NO: 46 - DNA, 24 bp, Artificial Sequence
  synthetic
caaaataaccacgcgaacgacgaa

SEQ ID NO: 47 - DNA, 28 bp, Artificial Sequence
  synthetic
tcgtagagggcggatattaaattagcgg

SEQ ID NO: 48 - DNA, 26 bp, Artificial Sequence
  synthetic
cgaccgaaaaacaacgtttattcgct

SEQ ID NO: 49 - DNA, 25 bp, Artificial Sequence
  synthetic
ttttcggcggagtcgggattttttc

SEQ ID NO: 50 - DNA, 20 bp, Artificial Sequence
  synthetic
gatctccgctcgcctcgacg

SEQ ID NO: 51 - DNA, 24 bp, Artificial Sequence
  synthetic
gttgcggggtatcgggtttgattc

SEQ ID NO: 52 - DNA, 25 bp, Artificial Sequence
  synthetic
cctaccctctcccaaaaaacgcgtc

SEQ ID NO: 53 - DNA, 25 bp, Artificial Sequence
  synthetic
cggatagcggagtttcgagtcgttc

SEQ ID NO: 54 - DNA, 26 bp, Artificial Sequence
  synthetic
aaacgtctccttaattccccgcgctt

SEQ ID NO: 55 - DNA, 25 bp, Artificial Sequence
  synthetic
taagcgcggggaattaaggagacgt

SEQ ID NO: 56 - DNA, 27 bp, Artificial Sequence
  synthetic
cgaaaacgcgaaactaaaatcgacgta

SEQ ID NO: 57 - DNA, 20 bp, Artificial Sequence
  synthetic
gttggcggaggcggttcgag

SEQ ID NO: 58 - DNA, 20 bp, Artificial Sequence
  synthetic
cgacgtacgtccctacgaaa

SEQ ID NO: 59 - DNA, 25 bp, Artificial Sequence
  synthetic
atttgttttgtcgggaaggggacgg

SEQ ID NO: 60 - DNA, 25 bp, Artificial Sequence
  synthetic
caaataacccaaactcctatacgca

SEQ ID NO: 61 - DNA, 27 bp, Artificial Sequence
  synthetic
cgtttgagaattttaggagttgagcgg

SEQ ID NO: 62 - DNA, 25 bp, Artificial Sequence
  synthetic
cgaaacaacgaaactaaaacgcgct

SEQ ID NO: 63 - DNA, 24 bp, Artificial Sequence
  synthetic
gaggagaggtaggagaggttacgg

SEQ ID NO: 64 - DNA, 22 bp, Artificial Sequence
  synthetic
aataattcccactctacgcgaa

SEQ ID NO: 65 - DNA, 29 bp, Artificial Sequence
  synthetic
tcgaaaagataattaaaaatcgtacgcgt

SEQ ID NO: 66 - DNA, 25 bp, Artificial Sequence
  synthetic
cgaaaacccatttaaccaaaccgaa

SEQ ID NO: 67 - DNA, 24 bp, Artificial Sequence
  synthetic
ggtggttcggcgtaattaaagcgt

SEQ ID NO: 68 - DNA, 25 bp, Artificial Sequence
  synthetic
cgtacgccttccgcaaatatacgaa

SEQ ID NO: 69 - DNA, 25 bp, Artificial Sequence
  synthetic
ttttacgtagaaataaaggacgcgt

SEQ ID NO: 70 - DNA, 25 bp, Artificial Sequence
  synthetic
aaaaaaccgaaaccccaaaaacgac

SEQ ID NO: 71 - DNA, 25 bp, Artificial Sequence
  synthetic
gttcgttttcgataagcgtttcggt

SEQ ID NO: 72 - DNA, 20 bp, Artificial Sequence
  synthetic
aatccccactccctccgata

SEQ ID NO: 73 - DNA, 25 bp, Artificial Sequence
  synthetic
aaggtattgtcgcgggttcgttcgt

SEQ ID NO: 74 - DNA, 32 bp, Artificial Sequence
  synthetic
taaaataaatcatttaacccataataaccgaa

SEQ ID NO: 75 - DNA, 31 bp, Artificial Sequence
  synthetic
ggtttaggtcgatgaaggttaggttcgcgta

SEQ ID NO: 76 - DNA, 24 bp, Artificial Sequence
  synthetic
ctatcgaccaacatcgcgctaccg

SEQ ID NO: 77 - DNA, 28 bp, Artificial Sequence
  synthetic
ttagtttcgttttcgtttaggtgcgttg

SEQ ID NO: 78 - DNA, 25 bp, Artificial Sequence
  synthetic
aaatacgactcccgataacccgcga

SEQ ID NO: 79 - DNA, 29 bp, Artificial Sequence
  synthetic
cggttgtagggaggatttcgaggaagttc

SEQ ID NO: 80 - DNA, 20 bp, Artificial Sequence
  synthetic
acgccaaaaaacccaacgca

SEQ ID NO: 81 - DNA, 21 bp, Artificial Sequence
  synthetic
tcgggatagaggcgagagttc

SEQ ID NO: 82 - DNA, 25 bp, Artificial Sequence
  synthetic
gaaacccgcttttctttttccaaac

SEQ ID NO: 83 - DNA, 25 bp, Artificial Sequence
  synthetic
cgagtgcgttttttattagcgtagc

SEQ ID NO: 84 - DNA, 29 bp, Artificial Sequence
  synthetic
gttaaaaaaactattaacgataacgccgc

SEQ ID NO: 85 - DNA, 26 bp, Artificial Sequence
  synthetic
ggttcgaaggtatagtgagtttcgtc

SEQ ID NO: 86 - DNA, 21 bp, Artificial Sequence
  synthetic
aaaacccaccgaatccttcga

SEQ ID NO: 87 - DNA, 16 bp, Artificial Sequence
  synthetic
ttcgttcgtcggggcg

SEQ ID NO: 88 - DNA, 32 bp, Artificial Sequence
  synthetic
gtttcctacttaccaaaataaaaaaaacgaaa

SEQ ID NO: 89 - DNA, 25 bp, Artificial Sequence
  synthetic
tgttgggtttttttcgttggagatc

SEQ ID NO: 90 - DNA, 18 bp, Artificial Sequence
  synthetic
gaaccccgcgtacttccg

SEQ ID NO: 91 - DNA, 22 bp, Artificial Sequence
  synthetic
cgcggttatggttagtagcggc

SEQ ID NO: 92 - DNA, 18 bp, Artificial Sequence
  synthetic
gaacgacgcgaactccga

SEQ ID NO: 93 - DNA, 23 bp, Artificial Sequence
  synthetic
ggcgttaggaggtttagcgtatc

SEQ ID NO: 94 - DNA, 25 bp, Artificial Sequence
  synthetic
ctccaaaaccttctccctaaatcga

SEQ ID NO: 95 - DNA, 20 bp, Artificial Sequence
  synthetic
agttcgcgttatcgtcgtcg

SEQ ID NO: 96 - DNA, 17 bp, Artificial Sequence
  synthetic
ccgcccacgtaaaaccg

SEQ ID NO: 97 - DNA, 25 bp, Artificial Sequence
  synthetic
gttttagaggtcgttaagtttcggc

SEQ ID NO: 98 - DNA, 22 bp, Artificial Sequence
  synthetic
ataaaaccgcaaaaaccaccga

SEQ ID NO: 99 - DNA, 20 bp, Artificial Sequence
  synthetic
tttcgttggttttcgagcgc

SEQ ID NO: 100 - DNA, 23 bp, Artificial Sequence
  synthetic
cgaaatatacgtacgccttccgc

SEQ ID NO: 101 - DNA, 21 bp, Artificial Sequence
  synthetic
gtaggaggcgaggtttaagcg

SEQ ID NO: 102 - DNA, 16 bp, Artificial Sequence
  synthetic
cccgccccctaatccg

SEQ ID NO: 103 - DNA, 20 bp, Artificial Sequence
  synthetic
ggttcgttcgttcgttcgtc

SEQ ID NO: 104 - DNA, 24 bp, Artificial Sequence
  synthetic
aaaacttactaaaaatcgccccga

SEQ ID NO: 105 - DNA, 25 bp, Artificial Sequence
  synthetic
attttgacgtttcgtttttgatcgc

SEQ ID NO: 106 - DNA, 23 bp, Artificial Sequence
  synthetic
gaaacaacgaaactaaaacgcgc

SEQ ID NO: 107 - DNA, 20 bp, Artificial Sequence
  synthetic
tcggcgttatcgcggttatc

SEQ ID NO: 108 - DNA, 25 bp, Artificial Sequence
  synthetic
aaaacaaaaaactttctcaacgcga

SEQ ID NO: 109 - DNA, 20 bp, Artificial Sequence
  synthetic
aaggtattgtcgcgggttcg

SEQ ID NO: 110 - DNA, 18 bp, Artificial Sequence
  synthetic
aatccccactccctccga

SEQ ID NO: 111 - DNA, 30 bp, Artificial Sequence
  synthetic
tgttttttaaatgatttaacgtcgggattc

SEQ ID NO: 112 - DNA, 19 bp, Artificial Sequence
  synthetic
cgaacgccgaacacttcga

SEQ ID NO: 113 - DNA, 20 bp, Artificial Sequence
  synthetic
cggaatttcggttttcgcgg

SEQ ID NO: 114 - DNA, 25 bp, Artificial Sequence
  synthetic
ccgaaaaactttcaaacactaaccg

SEQ ID NO: 115 - DNA, 20 bp, Artificial Sequence
  synthetic
ggagcggatagcggagtttc

SEQ ID NO: 116 - DNA, 25 bp, Artificial Sequence
  synthetic
ggggcgatagtatttaggtcgtcga

SEQ ID NO: 117 - DNA, 24 bp, Artificial Sequence
  synthetic
acgaaaacgcgaaactaaaatcga

SEQ ID NO: 118 - DNA, 25 bp, Artificial Sequence
  synthetic
gatctcaacacaactcgttactcga

SEQ ID NO: 119 - DNA, 25 bp, Artificial Sequence
  synthetic
ggtttttggtgatatcgtattcgcg

SEQ ID NO: 120 - DNA, 22 bp, Artificial Sequence
  synthetic
cgggggcgagatagatgatttc

SEQ ID NO: 121 - DNA, 22 bp, Artificial Sequence
  synthetic
gactaactccctaaccgaaccg

SEQ ID NO: 122 - DNA, 19 bp, Artificial Sequence
  synthetic
tctcaaaatcacccccgcg

SEQ ID NO: 123 - DNA, 26 bp, Artificial Sequence
  synthetic
gcgtggtttttattatagttcggtcg

SEQ ID NO: 124 - DNA, 19 bp, Artificial Sequence
  synthetic
cgccgtacacgaataccga

SEQ ID NO: 125 - DNA, 18 bp, Artificial Sequence
  synthetic
ggagtttcgtagcgggcg

SEQ ID NO: 126 - DNA, 20 bp, Artificial Sequence
  synthetic
cctcccaaaaaaacgacgcg

SEQ ID NO: 127 - DNA, 21 bp, Artificial Sequence
  synthetic
ggaatggtttcgtagttgcgc

SEQ ID NO: 128 - DNA, 20 bp, Artificial Sequence
  synthetic
ggtcggtgcgttcgtttttc

SEQ ID NO: 129 - DNA, 28 bp, Artificial Sequence
  synthetic
cgaattaactatcgaaaacgatttccgc

SEQ ID NO: 130 - DNA, 24 bp, Artificial Sequence
  synthetic
tggtatttttcggggaaagtttcg

SEQ ID NO: 131 - DNA, 24 bp, Artificial Sequence
  synthetic
cacgcgtctaaccataaactacac

SEQ ID NO: 132 - DNA, 19 bp, Artificial Sequence
  synthetic
cccgactcgaaaacctccg

SEQ ID NO: 133 - DNA, 18 bp, Artificial Sequence
  synthetic
tcgttgggtgttcgtcgc

SEQ ID NO: 134 - DNA, 23 bp, Artificial Sequence
  synthetic
ttacttactacctccgactccgc

SEQ ID NO: 135 - DNA, 27 bp, Artificial Sequence
  synthetic
tcgtattcgggtttatttcgtttttcg

SEQ ID NO: 136 - DNA, 17 bp, Artificial Sequence
  synthetic
cgcgccctctaacgacc

SEQ ID NO: 137 - DNA, 34 bp, Artificial Sequence
  synthetic
aggccacggacgcgaattcggttttagtgttgga

SEQ ID NO: 138 - DNA, 28 bp, Artificial Sequence
  synthetic
cgcgccgaggcgcggtcggtttgtttat

SEQ ID NO: 139 - DNA, 34 bp, Artificial Sequence
  synthetic
aggccacggacgcgtttttcggatttggttttcg

SEQ ID NO: 140 - DNA, 26 bp, Artificial Sequence
  synthetic
cgcgccgagggcgcgtttttgcgttt

SEQ ID NO: 141 - DNA, 37 bp, Artificial Sequence
  synthetic
aggccacggacgcgcgttttttttaaatttttgtgag

SEQ ID NO: 142 - DNA, 30 bp, Artificial Sequence
  synthetic
cgcgccgaggcgtttcggttacgtttaagc

SEQ ID NO: 143 - DNA, 30 bp, Artificial Sequence
  synthetic
aggccacggacgcgcgtcgagttttttcgg

SEQ ID NO: 144 - DNA, 27 bp, Artificial Sequence
  synthetic
cgcgccgagggcgacccgtcgaataac

SEQ ID NO: 145 - DNA, 36 bp, Artificial Sequence
  synthetic
aggccacggacgcgattcgtagtattcgggttatag

SEQ ID NO: 146 - DNA, 29 bp, Artificial Sequence
  synthetic
cgcgccgaggcgggattcgcggtttttag

SEQ ID NO: 147 - DNA, 32 bp, Artificial Sequence
  synthetic
aggccacggacggatccgaactcctaaacccg

SEQ ID NO: 148 - DNA, 35 bp, Artificial Sequence
  synthetic
cgcgccgaggcgcgcgtttttttttttttataaat

SEQ ID NO: 149 - DNA, 30 bp, Artificial Sequence
  synthetic
aggccacggacgcggtcgaggtttttacgg

SEQ ID NO: 150 - DNA, 29 bp, Artificial Sequence
  synthetic
cgcgccgaggcgtgatcgtcgttttgttg

SEQ ID NO: 151 - DNA, 34 bp, Artificial Sequence
  synthetic
aggccacggacggttcgttttcgataagcgtttc

SEQ ID NO: 152 - DNA, 30 bp, Artificial Sequence
  synthetic
cgcgccgaggcgtagggagttttcgattcg

SEQ ID NO: 153 - DNA, 33 bp, Artificial Sequence
  synthetic
aggccacggacggcgcctaatatcgctaaaaac

SEQ ID NO: 154 - DNA, 31 bp, Artificial Sequence
  synthetic
cgcgccgaggcgagtcgttcgtagagtaagc

SEQ ID NO: 155 - DNA, 30 bp, Artificial Sequence
  synthetic
aggccacggacgcggcgatttgagtgttgt

SEQ ID NO: 156 - DNA, 27 bp, Artificial Sequence
  synthetic
cgcgccgagggaacctacgacgccctc

SEQ ID NO: 157 - DNA, 33 bp, Artificial Sequence
  synthetic
aggccacggacgcgcgggtttttatcgattttc

SEQ ID NO: 158 - DNA, 25 bp, Artificial Sequence
  synthetic
cgcgccgagggcggtaggttggggt

SEQ ID NO: 159 - DNA, 31 bp, Artificial Sequence
  synthetic
aggccacggacggaaaccctatactcccccg

SEQ ID NO: 160 - DNA, 29 bp, Artificial Sequence
  synthetic
cgcgccgaggcgttgttttcgcgttttcg

SEQ ID NO: 161 - DNA, 31 bp, Artificial Sequence
  synthetic
aggccacggacgcgcgtttttcgttcgattg

SEQ ID NO: 162 - DNA, 35 bp, Artificial Sequence
  synthetic
cgcgccgagggaaacgaactaaaactactcgatcc

SEQ ID NO: 163 - DNA, 28 bp, Artificial Sequence
  synthetic
aggccacggacgcgcggttaggtttgcg

SEQ ID NO: 164 - DNA, 26 bp, Artificial Sequence
  synthetic
cgcgccgaggcgcggttaggtttgcg

SEQ ID NO: 165 - DNA, 28 bp, Artificial Sequence
  synthetic
cgcgccgaggcgcgaaacgaaaacacct

SEQ ID NO: 166 - DNA, 29 bp, Artificial Sequence
  synthetic
aggccacggacggcgattcccaaaacccg

SEQ ID NO: 167 - DNA, 26 bp, Artificial Sequence
  synthetic
cgcgccgaggcggcggttttcgaagc

SEQ ID NO: 168 - DNA, 143 bp, Artificial Sequence
  synthetic
agttgcggcccgggacagaggcgagagctcgaactcggtcccagtgctggagcatccccaggagagaggggggcccgaggacttggggcttctgcttggaaacattcgtctggaaaaagaaaagcgggccccgggctcaggac

SEQ ID NO: 169 - DNA, 143 bp, Artificial Sequence
  synthetic
agttgcggttcgggatagaggcgagagttcgaattcggttttagtgttggagtatttttaggagagaggggggttcgaggatttggggtttttgtttggaaatattcgtttggaaaaagaaaagcgggtttcgggtttaggat

SEQ ID NO: 170 - DNA, 131 bp, Artificial Sequence
  synthetic
gttcgggatagaggcgagagttcgaatacggttttagtgttggagtatttttaggagagaggggggttcgaggatttggggtttttgtttggaaatattcgtttggaaaaagaaaagcgggtttcgggttt

SEQ ID NO: 171 - DNA, 33 bp, Artificial Sequence
  synthetic
aggccacggacggaattcggttttagtgttgga

SEQ ID NO: 172 - DNA, 200 bp, Artificial Sequence
  synthetic
cttctaccagaacaaccggcgctacggcgtgtcccgcgacgacgcgcagctgagcggactccccagcgcgctgcgccaccctgtcaacgagtgcgccccctaccagcgcagcgcggccggcctgcccatcgcgccctgcggcgccatcgccaacagcctcttcaacgactccttctcgctttggcaccagcgccagcccg

SEQ ID NO: 173 - DNA, 200 bp, Artificial Sequence
  synthetic
tttttattagaataatcggcgttacggcgtgtttcgcgacgacgcgtagttgagcggattttttagcgcgttgcgttattttgttaacgagtgcgttttttattagcgtagcgcggtcggtttgtttatcgcgttttgcggcgttatcgttaatagtttttttaacgattttttttcgttttggtattagcgttagttcg

SEQ ID NO: 174 - DNA, 88 bp, Artificial Sequence
  synthetic
ttaacgagtgcgttttttattagcgtagcgcggtcggtttgtttatcgcgttttgcggcgttatcgttaatagtttttttaacgattt

SEQ ID NO: 175 - DNA, 230 bp, Artificial Sequence
  synthetic
gccttccctgcaagtgaaacttctttccaagtggacgctttgcagaaatccggcccccgagcagagtgtaggtccgaaggcacagtgagccccgccgtctcccggacctggcttccgcggactgggaagaagaccccccgcctggccaggccgaaggattcggtgggctcctcagtggagcccacggtcccgacacccgcggtcgtggggcccctgggcctgtgcacccg

SEQ ID NO: 176 - DNA, 230 bp, Artificial Sequence
  synthetic
gttttttttgtaagtgaaatttttttttaagtggacgttttgtagaaattcggttttcgagtagagtgtaggttcgaaggtatagtgagtttcgtcgtttttcggatttggttttcgcggattgggaagaagatttttcgtttggttaggtcgaaggattcggtgggttttttagtggagtttacggtttcgatattcgcggtcgtggggtttttgggtttgtgtattcg

SEQ ID NO: 177 - DNA, 114 bp, Artificial Sequence
  synthetic
agtgtaggttcgaaggtatagtgagtttcgtcgtttttcggatttggttttcgcggattgggaagaagatttttcgtttggttaggtcgaaggattcggtgggttttttagtgg

SEQ ID NO: 178 - DNA, 68 bp, Artificial Sequence
  synthetic
cggggcgcgcgctcctgcgccccctccccttagcccccgccccccccactctggcaagcaggaaacgc

SEQ ID NO: 179 - DNA, 68 bp, Artificial Sequence
  synthetic
cggggcgcgcgtttttgcgttttttttttttagttttcgttttttttattttggtaagtaggaaacgt

SEQ ID NO: 180 - DNA, 88 bp, Artificial Sequence
  synthetic
gcccgcccgccggggcgcgcgctcctgcgccccctccccttagcccccgccccccccactctggcaagcaggaaacgcgtggccctaa

SEQ ID NO: 181 - DNA, 88 bp, Artificial Sequence
  synthetic
gttcgttcgtcggggcgcgcgtttttgcgttttttttttttagttttcgttttttttattttggtaagtaggaaacgtgtggttttaa

SEQ ID NO: 182 - DNA, 83 bp, Artificial Sequence
  synthetic
gttcgttcgtcggggcgcgcgtttttgcgttttttttttttagttttcgttttttttattttggtaagtaggaaacgtgtggt

SEQ ID NO: 183 - DNA, 88 bp, Artificial Sequence
  synthetic
ttagggccacgcgtttcctgcttgccagagtggggggggcgggggctaaggggagggggcgcaggagcgcgcgccccggcgggcgggc

SEQ ID NO: 184 - DNA, 88 bp, Artificial Sequence
  synthetic
ttagggttatgcgttttttgtttgttagagtggggggggcgggggttaaggggagggggcgtaggagcgcgcgtttcggcgggcgggt

SEQ ID NO: 185 - DNA, 157 bp, Artificial Sequence
  synthetic
ggcaggcccccggccaggtgcatgggaggaagcacggagaatttacaagcctctcgattcctcagtccagacgctgttgggtcccctccgctggagatcgcgcttcccccaaatctttgtgagcgttgcggaagcacgcggggtccgggtcgctgag

SEQ ID NO: 186 - DNA, 157 bp, Artificial Sequence
  synthetic
ggtaggttttcggttaggtgtatgggaggaagtacggagaatttataagtttttcgattttttagtttagacgttgttgggtttttttcgttggagatcgcgttttttttaaatttttgtgagcgttgcggaagtacgcggggttcgggtcgttgag

SEQ ID NO: 187 - DNA, 76 bp, Artificial Sequence
  synthetic
tgttgggtttttttcgttggagatcgcgttttttttaaatttttgtgagcgttgcggaagtacgcggggttcgggt

SEQ ID NO: 188 - DNA, 149 bp, Artificial Sequence
  synthetic
cggcggcttgtccttctacggcctggcgggtggcgccagcctcttctaccgcttcctggcagccttcctcgagccgctctaccccgcggtcatggtcagcagcggcgcctcggccacgctcaagcggcgcccggagcccgcgccgcccg

SEQ ID NO: 189 - DNA, 149 bp, Artificial Sequence
  synthetic
cggcggtttgtttttttacggtttggcgggtggcgttagttttttttattgttttttggtagtttttttcgagtcgttttatttcgcggttatggttagtagcggcgtttcggttacgtttaagcggcgttcggagttcgcgtcgttcg

SEQ ID NO: 190 - DNA, 68 bp, Artificial Sequence
  synthetic
tttcgcggttatggttagtagcggcgtttcggttacgtttaagcggcgttcggagttcgcgtcgttcg

SEQ ID NO: 191 - DNA, 86 bp, Artificial Sequence
  synthetic
cggcgccaggaggcccagcgcaccgcgccgagcttcctcggagtcctccctacagccgactcagggagaaggtcctggagggcccg

SEQ ID NO: 192 - DNA, 86 bp, Artificial Sequence
  synthetic
cggcgttaggaggtttagcgtatcgcgtcgagttttttcggagttttttttatagtcgatttagggagaaggttttggagggttcg

SEQ ID NO: 193 - DNA, 86 bp, Artificial Sequence; identical to SEQ ID NO: 192
  synthetic

SEQ ID NO: 194 - DNA, 95 bp, Artificial Sequence
  synthetic
cggaagagcccgcgccaccgccgccgccaccaccaacacgttgcaaggagtcattcgacgggccgcggccccacgtgggcggggcaggccacccg

SEQ ID NO: 195 - DNA, 95 bp, Artificial Sequence
  synthetic
cggaagagttcgcgttatcgtcgtcgttattattaatacgttgtaaggagttattcgacgggtcgcggttttacgtgggcggggtaggttattcg

SEQ ID NO: 196 - DNA, 95 bp, Artificial Sequence; identical to SEQ ID NO: 195
  synthetic

SEQ ID NO: 197 - DNA, 227 bp, Artificial Sequence
  synthetic
gcctcctcgaggcggaggcccctgttccagaggccgccaagccccggcgattcgcagtacccgggccacagggcccgccatgggcgctgccaggcctgaggtcgctgtatccggtggcctctgcggttccaccacccccaccgcgggatgggcggtctctggggaacaccgcccgcccgcccgcatgaccgcgctggccttggagagggagccaggggacccaaccg

SEQ ID NO: 198 - DNA, 227 bp, Artificial Sequence
  synthetic
gttttttcgaggcggaggtttttgttttagaggtcgttaagtttcggcgattcgtagtattcgggttatagggttcgttatgggcgttgttaggtttgaggtcgttgtattcggtggtttttgcggttttattatttttatcgcgggatgggcggtttttggggaatatcgttcgttcgttcgtatgatcgcgttggttttggagagggagttaggggatttaatcg

SEQ ID NO: 199 - DNA, 126 bp, Artificial Sequence
  synthetic
tgttttagaggtcgttaagtttcggcgattcgtagtattcgggttatagggttcgttatgggcgttgttaggtttgaggtcgttgtattcggtggtttttgcggttttattatttttatcgcggga

SEQ ID NO: 200 - DNA, 135 bp, Artificial Sequence
  synthetic
cggccctgcggattccgttggtttccgagcgcgggatccgcggcctctagtgggcgcagggcaggtggctcggcgtaaccaaagcgccttctctggacctctccgcatatctgcggaaggcgcacgcacatcccg

SEQ ID NO: 201 - DNA, 135 bp, Artificial Sequence
  synthetic
cggttttgcggatttcgttggttttcgagcgcgggattcgcggtttttagtgggcgtagggtaggtggttcggcgtaattaaagcgttttttttggattttttcgtatatttgcggaaggcgtacgtatatttcg

SEQ ID NO: 202 - DNA, 131 bp, Artificial Sequence
  synthetic
tttgcggatttcgttggttttcgagcgcgggattcgcggtttttagtgggcgtagggtaggtggttcggcgtaattaaagcgttttttttggattttttcgtatatttgcggaaggcgtacgtatatttcg

SEQ ID NO: 203 - DNA, 181 bp, Artificial Sequence
  synthetic
cgggcagggcggagagggcgaggtcagaggggctgtctccgtgctgggaaggggtgtggcctgcgggggttggagccctagcaggaggcgaggtctaagcgggtggggcgaagtggattctgaattaggtaggggcgggcctaggagtccggaccggatcagggggcggggccggagaggc

SEQ ID NO: 204 - DNA, 181 bp, Artificial Sequence
  synthetic
cgggtagggcggagagggcgaggttagaggggttgttttcgtgttgggaaggggtgtggtttgcgggggttggagttttagtaggaggcgaggtttaagcgggtggggcgaagtggattttgaattaggtaggggcgggtttaggagttcggatcggattagggggcggggtcggagaggt

SEQ ID NO: 205 - DNA, 102 bp, Artificial Sequence
  synthetic
tttagtaggaggcgaggtttaagcgggtggggcgaagtggattttgaattaggtaggggcgggtttaggagttcggatcggattagggggcggggtcggaga

SEQ ID NO: 206 - DNA, 170 bp, Artificial Sequence
  synthetic
cggcccgctcgctcgctcgccgcgcgctctcttcctcttacaaactttcgggttctgcagtcgacaagaaaccggggcgaccctcagcaagtcttccccgtgcaggctggggggaactgaaggggacgggacgggtgagtaggccgaggggccaggacagagagggcagc

SEQ ID NO: 207 - DNA, 170 bp, Artificial Sequence
  synthetic
cggttcgttcgttcgttcgtcgcgcgtttttttttttttataaattttcgggttttgtagtcgataagaaatcggggcgatttttagtaagtttttttcgtgtaggttggggggaattgaaggggacgggacgggtgagtaggtcgaggggttaggatagagagggtagt

SEQ ID NO: 208 - DNA, 100 bp, Artificial Sequence
  synthetic
cggttcgttcgttcgttcgtcgcgcgtttttttttttttataaattttcgggttttgtagtcgataagaaatcggggcgatttttagtaagtttttttcg

SEQ ID NO: 209 - DNA, 124 bp, Artificial Sequence
  synthetic
cggcctcgcctgagaactccaggagctgagcggattttgacgccccgcccttgaccgcggtcgaggcccccacggcgccccagcgcgtctcagccccgctgtcccgcccgaactccgaaccccg

SEQ ID NO: 210 - DNA, 124 bp, Artificial Sequence
  synthetic
cggtttcgtttgagaattttaggagttgagcggattttgacgtttcgtttttgatcgcggtcgaggtttttacggcgttttagcgcgttttagtttcgttgtttcgttcgaatttcgaatttcg

SEQ ID NO: 211 - DNA, 84 bp, Artificial Sequence
  synthetic
agcggattttgacgtttcgtttttgatcgcggtcgaggtttttacggcgttttagcgcgttttagtttcgttgtttcgttcgaa

SEQ ID NO: 212 - DNA, 449 bp, Artificial Sequence
  synthetic
ccggcgctatcgcggccatcgtgatcgccgccctgctggccacctgcgtggtgctggcgctcgtggtcgtcgcgctgagaaagttttctgcctcctgaagcgaataaaggggccgcgcccggccgcggcgcgactcggctgcactcctcacgcgcctgtatgtccgcgcgtgcgtgtccgcgcatgcaggtgtgccagagcgtgagcgcgcgcaggcgagcgctcagggcgggggtgcgcgcggggccgagggtgggtgccgtgcacgcgcgcgggtccggaggcgcgcccgaattccccgcaggggcgccggggcgtgcgtgagcgcacagcatgtccgagcccgcctgcgtgtggcgtgcacctgagcgcgcgcagggccgcgcggctcggcgtccccgtgcaccacggctggagtgcctcaggagcgcgccgcatgtgcgtgcccg

SEQ ID NO: 213 - DNA, 201 bp, Artificial Sequence
  synthetic
ccggcgctatcgcggccatcgtgatcgccgccctgctggccacctgcgtggtgctggcgctcgtggtcgtcgcgctgagaaagttttctgcctcctgaagcgaataaaggggccgcgcccggccgcggcgcgactcggctgcactcctcacgcgcctgtatgtccgcgcgtgcgtgtccgcgcatgcaggtgtgccagagc

SEQ ID NO: 214 - DNA, 201 bp, Artificial Sequence
  synthetic
tcggcgttatcgcggttatcgtgatcgtcgttttgttggttatttgcgtggtgttggcgttcgtggtcgtcgcgttgagaaagttttttgttttttgaagtgaataaaggggtcgcgttcggtcgcggcgcgattcggttgtattttttatgcgtttgtatgttcgcgcgtgcgtgttcgcgtatgtaggtgtgttagagt

SEQ ID NO: 215 - DNA, 101 bp, Artificial Sequence
  synthetic
tcggcgttatcgcggttatcgtgatcgtcgttttgttggttatttgcgtggtgttggcgttcgtggtcgtcgcgttgagaaagttttttgttttttgaagt

SEQ ID NO: 216 - DNA, 107 bp, Artificial Sequence
  synthetic
aaggcactgccgcgggtccgcccgcccccgataagcgcctcggccattatgggccaaatgattcattttaatttggcaatttcaccggagggagtggggactggttg

SEQ ID NO: 217 - DNA, 107 bp, Artificial Sequence
  synthetic
aaggtattgtcgcgggttcgttcgttttcgataagcgtttcggttattatgggttaaatgatttattttaatttggtaattttatcggagggagtggggattggttg

SEQ ID NO: 218 - DNA, 107 bp, Artificial Sequence; identical to SEQ ID NO: 217
  synthetic

SEQ ID NO: 219 - DNA, 217 bp, Artificial Sequence
  synthetic
gatgaggcccggggcgccccgtgtcgccgccggagaggcaccctgccccgggagcatcaccgctttctggacctgtccttcaaatgatccaacgccgggactcgcagggagccctcgacccgcccagctgagcctgccccaaactacagctcccgaagtgctcggcgttcggagcgggcttccgttcccagcgtgcccagtgagccgggcgccggaa

SEQ ID NO: 220 - DNA, 217 bp, Artificial Sequence
  synthetic
gatgaggtttggggcgtttcgtgtcgtcgtcggagaggtattttgtttcgggagtattatcgttttttggatttgttttttaaatgatttaacgtcgggattcgtagggagttttcgattcgtttagttgagtttgttttaaattatagttttcgaagtgttcggcgttcggagcgggttttcgtttttagcgtgtttagtgagtcgggcgtcggaa

SEQ ID NO: 221 - DNA, 106 bp, Artificial Sequence
  synthetic
atttgttttttaaatgatttaacgtcgggattcgtagggagttttcgattcgtttagttgagtttgttttaaattatagttttcgaagtgttcggcgttcggagcg

SEQ ID NO: 222 - DNA, 75 bp, Artificial Sequence
  synthetic
gccggaactccggttttcgcggttctcagcgatattaggcgcggccagtgtctgaaagctcctcggggttacgtc

SEQ ID NO: 223 - DNA, 75 bp, Artificial Sequence
  synthetic
gtcggaatttcggttttcgcggtttttagcgatattaggcgcggttagtgtttgaaagtttttcggggttacgtt

SEQ ID NO: 224 - DNA, 75 bp, Artificial Sequence; identical to SEQ ID NO: 223
  synthetic

SEQ ID NO: 225 - DNA, 361 bp, Artificial Sequence
  synthetic
cgggcgctgggctttttcgacttttcctttaaaaagaaaaaagtttttcaagctgtaggttccaagaacaggcaggaggggggagaagggggggggggttgcagaaaaggcgcctggtcggttatgagtcacaagtgagttataaaagggtcgcacgttcgcaggcgcgggcttcctgtgcgcggccgagcccgggcccagcgccgcctgcagcctcgggaagggagcggatagcggagccccgagccgcccgcagagcaagcgcggggaaccaaggagacgctcctggcactgcaggtacgccgacttcagtctcgcgctcccgcccgcctttcctctcttgaacgtggcagggacgccg

SEQ ID NO: 226 - DNA, 161 bp, Artificial Sequence
  synthetic
gcgccgcctgcagcctcgggaagggagcggatagcggagccccgagccgcccgcagagcaagcgcggggaaccaaggagacgctcctggcactgcaggtacgccgacttcagtctcgcgctcccgcccgcctttcctctcttgaacgtggcagggacgccg

SEQ ID NO: 227 - DNA, 161 bp, Artificial Sequence
  synthetic
gcgtcgtttgtagtttcgggaagggagcggatagcggagtttcgagtcgttcgtagagtaagcgcggggaattaaggagacgtttttggtattgtaggtacgtcgattttagtttcgcgttttcgttcgtttttttttttttgaacgtggtagggacgtcg

SEQ ID NO: 228 - DNA, 113 bp, Artificial Sequence
  synthetic
gaagggagcggatagcggagtttcgagtcgttcgtagagtaagcgcggggaattaaggagacgtttttggtattgtaggtacgtcgattttagtttcgcgttttcgttcgttt

SEQ ID NO: 229 - DNA, 114 bp, Artificial Sequence
  synthetic
cggtctcagcacagctcgttgctcgatgcctccattttctcggggcgacagcactcaggtcgccgatctgagggattcggagcttccccagctggggtgagcctggcgttcccg

SEQ ID NO: 230 - DNA, 114 bp, Artificial Sequence
  synthetic
cggttttagtatagttcgttgttcgatgtttttattttttcggggcgatagtatttaggtcgtcgatttgagggattcggagtttttttagttggggtgagtttggcgttttcg

SEQ ID NO: 231 - DNA, 114 bp, Artificial Sequence
  synthetic
cgggaacgccaggctcaccccagctggggaagctccgaatccctcagatcggcgacctgagtgctgtcgccccgagaaaatggaggcatcgagcaacgagctgtgctgagaccg

SEQ ID NO: 232 - DNA, 114 bp, Artificial Sequence
  synthetic
cgggaacgttaggtttattttagttggggaagtttcgaattttttagatcggcgatttgagtgttgtcgtttcgagaaaatggaggtatcgagtaacgagttgtgttgagatcg

SEQ ID NO: 233 - DNA, 97 bp, Artificial Sequence
  synthetic
ttttagttggggaagtttcgaattttttagatcggcgatttgagtgttgtcgtttcgagaaaatggaggtatcgagtaacgagttgtgttgagatcg

SEQ ID NO: 234 - DNA, 484 bp, Artificial Sequence
  synthetic
cggcagccacgggatcgcaacgcgggccgtcctgccctctggcccattaccttgttgagatgagggttcctggtgacatcgtatccgcgcttcttcaggggcacagggcgcgccgggccaggcttggagtggcagccttgggcgggcgcggtgggtgtccagcgcgggagggcgccgcaggcccggcccggccagggagccagccgcgtgcctgtccccagcgcggcacccatggtccttggcagacctggcacgggagagaaagcaaggtcagggcctccttccagccagccaggacccacgcgcgctgcggggcaccgggcctgatccggggacgcgccctctgggagagggcaggtgctcccaaagggttaaatgttccagcccccgccactcctcggctgccacctgctcggctccgcgcggggcgccaggcctagccaagagccaggcaaagcgaaaccaagccctggaagcagatccg

SEQ ID NO: 235 - DNA, 484 bp, Artificial Sequence
  synthetic
cggtagttacgggatcgtaacgcgggtcgttttgttttttggtttattattttgttgagatgagggtttttggtgatatcgtattcgcgtttttttaggggtatagggcgcgtcgggttaggtttggagtggtagttttgggcgggcgcggtgggtgtttagcgcgggagggcgtcgtaggttcggttcggttagggagttagtcgcgtgtttgtttttagcgcggtatttatggtttttggtagatttggtacgggagagaaagtaaggttagggtttttttttagttagttaggatttacgcgcgttgcggggtatcgggtttgattcggggacgcgttttttgggagagggtaggtgtttttaaagggttaaatgttttagttttcgttatttttcggttgttatttgttcggtttcgcgcggggcgttaggtttagttaagagttaggtaaagcgaaattaagttttggaagtagattcg

SEQ ID NO: 236 - DNA, 210 bp, Artificial Sequence
  synthetic
cggcagccacgggatcgcaacgcgggccgtcctgccctctggcccattaccttgttgagatgagggttcctggtgacatcgtatccgcgcttcttcaggggcacagggcgcgccgggccaggcttggagtggcagccttgggcgggcgcggtgggtgtccagcgcgggagggcgccgcaggcccggcccggccagggagccagccgcgtg

SEQ ID NO: 237 - DNA, 28 bp, Artificial Sequence
  synthetic
gttggggaagtttcgaattttttagatc

SEQ ID NO: 238 - DNA, 152 bp, Artificial Sequence
  synthetic
gatgagggtttttggtgatatcgtattcgcgtttttttaggggtatagggcgcgtcgggttaggtttggagtggtagttttgggcgggcgcggtgggtgtttagcgcgggagggcgtcgtaggttcggttcggttagggagttagtcgcgtg

SEQ ID NO: 239 - DNA, 218 bp, Artificial Sequence
  synthetic
cgggaccccttgcaggaactgtatccctgcctgcgacgggggcgagatagatgattccgcgggccctcaccgacctccggctctaaggaaggagtgaaggaagcgcgggggtgattttgagaaatccccttaaaaacttcctcccgaaagggggacctcccctggggacagacttgacagatcgcgagggtgggttcgcccgagtccgcgactagccg

SEQ ID NO: 240 - DNA, 218 bp, Artificial Sequence
  synthetic
cgggattttttgtaggaattgtatttttgtttgcgacgggggcgagatagatgatttcgcgggtttttatcgattttcggttttaaggaaggagtgaaggaagcgcgggggtgattttgagaaatttttttaaaaattttttttcgaaagggggattttttttggggatagatttgatagatcgcgagggtgggttcgttcgagttcgcgattagtcg

SEQ ID NO: 241 - DNA, 94 bp, Artificial Sequence
  synthetic
gcgacgggggcgagatagatgatttcgcgggtttttatcgattttcggttttaaggaaggagtgaaggaagcgcgggggtgattttgagaaatt

SEQ ID NO: 242 - DNA, 641 bp, Artificial Sequence
  synthetic
gggccagcttccacatctgcccagtggggcatttccccttgtcccacccagcgactggttgacaggaagcagggtgtctatggactggcggcgggcgccccttttcctcacctccctgcgcggccagcgggggcccggggtgtggtcaggtgggaggcccgcgccacgtgcggcaggaaccgcctggcgagtggaacccgagcttgcacggggacagctgatgcctttctggtcgctcctctagagctccgcgggctccggcaaacccccccgttagaacttgcggttccaacgcgcagcgccaaatctggcccttacttgacccgtcagcgcctccagtcgtccccatgtataaagtaggaccgaggagccacctcgccaagctttcactgcgcggaaacgcgcccatcaggctggaaaggcgtggttcctactacagctcggccgcggcaggctggggccgagaaccggcacccgtgcacggcggactaagacgagggtgagggacacgggtgggccgggagagccccgttcccatagccggcttccttgagcccctccccaagttctgggactccccaagttcccaccccgcaagccagtgtggtttcttcagatgaattttattcttactctccacg

SEQ ID NO: 243 - DNA, 200 bp, Artificial Sequence
  synthetic
tataaagtaggaccgaggagccacctcgccaagctttcactgcgcggaaacgcgcccatcaggctggaaaggcgtggttcctactacagctcggccgcggcaggctggggccgagaaccggcacccgtgcacggcggactaagacgagggtgagggacacgggtgggccgggagagccccgttcccatagccggcttcct

SEQ ID NO: 244 - DNA, 130 bp, Artificial Sequence
  synthetic
gatcgaggagttatttcgttaagtttttattgcgcggaaacgcgtttattaggttggaaaggcgtggtttttattatagttcggtcgcggtaggttggggtcgagaatcggtattcgtgtacggcggatt

SEQ ID NO: 245 - DNA, 130 bp, Artificial Sequence; identical to SEQ ID NO: 244
  synthetic

SEQ ID NO: 246 - DNA, 345 bp, Artificial Sequence
  synthetic
cggactcgcagcggtgacagcggaggcggggacgggggtggacaaactgcggatcaactctccgagaagacggccacctgccggaacctgccttcctccattccgcccaggagcctcgcagcggggcgctggcctgcggaaaggccacgggggagcacagggctccgcgccgccctcctgggagggtccggagtccagcgcaccccgagccctggtgggtgagcccggcgggaggcgcggcaggtgcggcgggagactggaccccgacctaccttcacaaagagctcgatctcggggtccctgtcgtccccgttagctgtcgccgagtctgtcatgccgttggcg

SEQ ID NO: 247 - DNA, 200 bp, Artificial Sequence
  synthetic
cggactcgcagcggtgacagcggaggcggggacgggggtggacaaactgcggatcaactctccgagaagacggccacctgccggaacctgccttcctccattccgcccaggagcctcgcagcggggcgctggcctgcggaaaggccacgggggagcacagggctccgcgccgccctcctgggagggtccggagtccagcg

SEQ ID NO: 248 - DNA, 199 bp, Artificial Sequence
  synthetic
cggattcgtagcggtgatagcggaggcggggacgggggtggataaattgtggattaatttttcgagaagacggttatttgtcggaatttgtttttttttatttcgtttaggagtttcgtagcgggcgttggtttgcggaaaggttacgggggagtatagggtttcgcgtcgtttttttgggagggttcggagtttagcg

SEQ ID NO: 249 - DNA, 89 bp, Artificial Sequence
  synthetic
tcgtttaggagtttcgtagcgggcgttggtttgcggaaaggttacgggggagtatagggtttcgcgtcgtttttttgggagggttcgga

SEQ ID NO: 250 - DNA, 106 bp, Artificial Sequence
  synthetic
cggccctgacctcagggcgctcaggtggaatggccccgcagctgcgcgctgctcccgcgcccccgccttgccccgcggaaatcgcccccgacagtcaacccgaccg

SEQ ID NO: 251 - DNA, 106 bp, Artificial Sequence
  synthetic
cggttttgattttagggcgtttaggtggaatggtttcgtagttgcgcgttgttttcgcgttttcgttttgtttcgcggaaatcgttttcgatagttaattcgatcg

SEQ ID NO: 252 - DNA, 88 bp, Artificial Sequence
  synthetic
gcgtttaggtggaatggtttcgtagttgcgcgttgttttcgcgttttcgttttgtttcgcggaaatcgttttcgatagttaattcgat

SEQ ID NO: 253 - DNA, 96 bp, Artificial Sequence
  synthetic
cgcgtctagccatggactgcacggcagtcgggcggggaacgcggagagcgagcgcaccgacctgtgagagaaggccaagaggtctgcgctgccgac

SEQ ID NO: 254 - DNA, 96 bp, Artificial Sequence
  synthetic
cgcgtttagttatggattgtacggtagtcgggcggggaacgcggagagcgagcgtatcgatttgtgagagaaggttaagaggtttgcgttgtcgat

SEQ ID NO: 255 - DNA, 102 bp, Artificial Sequence
  synthetic
gtcggtagcgtagattttttggtttttttttataggtcggtgcgttcgtttttcgcgtttttcgttcgattgtcgtgtagtttatggttagacgcgtcggat

SEQ ID NO: 256 - DNA, 102 bp, Artificial Sequence; identical to SEQ ID NO: 255
  synthetic

SEQ ID NO: 257 - DNA, 138 bp, Artificial Sequence
  synthetic
ggagggggccagggctggcatctcccggggaaagtttcgagcctggcagtgggaggagaccgtctgggcagagttgtgatgaactggggaccgagcagccccagctcgcctcggaggttcccgagccgggcgggagat

SEQ ID NO: 258 - DNA, 138 bp, Artificial Sequence
  synthetic
ggagggggttagggttggtatttttcggggaaagtttcgagtttggtagtgggaggagatcgtttgggtagagttgtgatgaattggggatcgagtagttttagttcgtttcggaggttttcgagtcgggcgggagat

SEQ ID NO: 259 - DNA, 138 bp, Artificial Sequence; identical to SEQ ID NO: 258
  synthetic

SEQ ID NO: 260 - DNA, 269 bp, Artificial Sequence
  synthetic
acctgcggcctgagttggccccgggcgcacgcgagggaaatggatgcagtgcccgtgaacttggggcctgaataattgataacaacccgctgggtgcccgtcgcgcggccaggcctgcggggagcggagccggaggcagcaagcaaaggaaacgcacgagccgggagcgcgggcgcgcggggccacgaaagaccgaccgactgtccgacgggcggtgggacaagaggacggatgctgtgggggggccccggaacccgccgccacaactc

SEQ ID NO: 261 - DNA, 269 bp, Artificial Sequence
  synthetic
atttgcggtttgagttggtttcgggcgtacgcgagggaaatggatgtagtgttcgtgaatttggggtttgaataattgataataattcgttgggtgttcgtcgcgcggttaggtttgcggggagcggagtcggaggtagtaagtaaaggaaacgtacgagtcgggagcgcgggcgcgcggggttacgaaagatcgatcgattgttcgacgggcggtgggataagaggacggatgttgtgggggggtttcggaattcgtcgttataattt

SEQ ID NO: 262 - DNA, 92 bp, Artificial Sequence
  synthetic
gataataattcgttgggtgttcgtcgcgcggttaggtttgcggggagcggagtcggaggtagtaagtaaaggaaacgtacgagtcgggagcg

SEQ ID NO: 263 - DNA, 100 bp, Artificial Sequence
  synthetic
gcgcactcgtacccgggttcacctcgcccctcgctcattcctcccgaccaaggcccatggtcagaggtgtcctcgccccgcggccgtcagagggcgcggc

SEQ ID NO: 264 - DNA, 100 bp, Artificial Sequence
  synthetic
gcgtattcgtattcgggtttatttcgtttttcgtttatttttttcgattaaggtttatggttagaggtgttttcgtttcgcggtcgttagagggcgcggt

SEQ ID NO: 265 - DNA, 100 bp, Artificial Sequence; identical to SEQ ID NO: 264
  synthetic

SEQ ID NO: 266 - DNA, 90 bp, Artificial Sequence
  synthetic
gccgcgccctctgacggccgcggggcgaggacacctctgaccatgggccttggtcgggaggaatgagcgaggggcgaggtgaacccgggt

SEQ ID NO: 267 - DNA, 90 bp, Artificial Sequence
  synthetic
gtcgcgttttttgacggtcgcggggcgaggatatttttgattatgggttttggtcgggaggaatgagcgaggggcgaggtgaattcgggt

SEQ ID NO: 268 - DNA, 85 bp, Artificial Sequence
  synthetic
gtcgcgttttttgacggtcgcggggcgaggatatttttgattatgggttttggtcgggaggaatgagcgaggggcgaggtgaatt

SEQ ID NO: 269 - DNA, 91 bp, Artificial Sequence
  synthetic
cgggaccagtaatgtcaaataaaacattggcttgtagacggacgcgggccttgggaatcgcgcgagcccagcgttagcccggggtttcacc

SEQ ID NO: 270 - DNA, 19 bp, Artificial Sequence
  synthetic
cgcgttttttgacggtcgc

SEQ ID NO: 271 - DNA, 18 bp, Artificial Sequence
  synthetic
cctcgctcattcctcccg

SEQ ID NO: 272 - DNA, 29 bp, Artificial Sequence
  synthetic
cgcgccgaggcggggcgaggatatttttg

SEQ ID NO: 273 - DNA, 91 bp, Artificial Sequence
  synthetic
cgggattagtaatgttaaataaaatattggtttgtagacggacgcgggttttgggaatcgcgcgagtttagcgttagttcggggttttatt

SEQ ID NO: 274 - DNA, 36 bp, Artificial Sequence
  synthetic
gtaatgttaaataaaatattggtttgtagacggacg

SEQ ID NO: 275 - DNA, 21 bp, Artificial Sequence
  synthetic
cgaactaacgctaaactcgcg

SEQ ID NO: 276 - DNA, 91 bp, Artificial Sequence; identical to SEQ ID NO: 273
  synthetic

SEQ ID NO: 277 - DNA, 167 bp, Artificial Sequence
  synthetic
gccgccgcggagccggccgggaggcagcgtttgttcgctggggttctgctagaagtcattctgtctcccgctggtttaatatccgccctctgcggtctccgctcgcctcggcggctcccgaagcgcccccgggagggtcccggctccgccgaaaactgcagcctgcc

SEQ ID NO: 278 - DNA, 167 bp, Artificial Sequence
  synthetic
gtcgtcgcggagtcggtcgggaggtagcgtttgttcgttggggttttgttagaagttattttgtttttcgttggtttaatattcgttttttgcggttttcgttcgtttcggcggttttcgaagcgttttcgggagggtttcggtttcgtcgaaaattgtagtttgtt

SEQ ID NO: 279 - DNA, 23 bp, Artificial Sequence
  synthetic
tttttgcggttttcgttcgtttc

SEQ ID NO: 280 - DNA, 19 bp, Artificial Sequence
  synthetic
cgaaaccgaaaccctcccg

SEQ ID NO: 281 - DNA, 95 bp, Artificial Sequence
  synthetic
ttcgttggtttaatattcgttttttgcggttttcgttcgtttcggcggttttcgaagcgttttcgggagggtttcggtttcgtcgaaaattgtag

(19) United States
(12) Patent Application Publication
(10) Pub. No.: US 2023/0151433 A1
(43) Pub. Date: May 18, 2023

(54) DETECTING MELANOMA

(71) Applicants: Mayo Foundation for Medical Education and Research, Rochester, MN (US); Exact Sciences Corporation, Madison, WI (US)

(72) Inventors: David A. Ahlquist, Rochester, MN (US); John B. Kisiel, Rochester, MN (US); William R. Taylor, Lake City, MN (US); Douglas W. Mahoney, Elgin, MN (US); Calise K. Berger, Rochester, MN (US); Hatim T. Allawi, Madison, WI (US); Viatcheslav Katerov, Madison, WI (US)

(21) Appl. No.: 17/921,711

(22) PCT Filed: May 4, 2021
(86) PCT No.: PCT/US2021/030638

§ 371 Date: Oct. 27, 2022

Related U.S. Application Data
(60) Provisional application No. 63/019,753, filed on May 4, 2020.

(51) Int. Cl. C12Q 1/6886; C12Q 1/6858

(52) U.S. Cl. CPC C12Q 1/6886; C12Q 1/6858; C12Q 2600/154

(57) ABSTRACT

Provided herein is technology for primary cutaneous melanoma screening and particularly, but not exclusively, to methods, compositions, and related uses for detecting the presence of primary cutaneous melanoma.

### CROSS-REFERENCE TO RELATED APPLICATION

The present application claims priority to U.S. Provisional Patent Application No. 63/019,753, filed May 4, 2020, which is hereby incorporated by reference in its entirety.

### FIELD OF INVENTION

Provided herein is technology for primary cutaneous melanoma screening and particularly, but not exclusively, to methods, compositions, and related uses for detecting the presence of primary cutaneous melanoma.

### BACKGROUND

Melanoma is a serious form of skin cancer in humans. It arises from the pigment cells (melanocytes), usually in the skin. The incidence of melanoma is increasing at the fastest rate of all cancers in the United States with a lifetime risk of 1 in 68. Although melanoma accounts for only 4% of all dermatologic cancers, it is responsible for 80% of all deaths from skin cancers. It has long been realized that recognition and diagnosis of melanoma, when it is early stage disease, is key to its cure. There are several types of melanoma, defined by where they first appear, including skin and eye melanoma and in rare instances in the GI tract or lymph nodes.

Primary cutaneous melanoma (PCM) is a common life-threatening malignancy responsible for 80% of skin cancer-related deaths (see, Miller, A. J. and M. C. Mihm Jr, New England Journal of Medicine, 2006. 355(1): p. 51-65). While the number of PCM diagnoses is ever increasing with a projected 150,000+ cases (in situ and invasive) in 2017 (American Cancer Society, 2017), the prognosis depends upon disease stage at diagnosis with a five-year overall survival rate of 98.1% for those with localized disease, 63.6% for those with regional disease, and 16.1% for those with distant metastases (American Cancer Society, 2017). Current disease surveillance of pre-metastatic PCM patients requires in-office visits with health care providers and interval PET/CT imaging. The cost of care already exceeds the resources of many patients with payers unable or unwilling to reimburse PET/CT. Lower socioeconomic status (SES) as evidenced by Medicaid insurance has been shown to delay melanoma care substantially (see, Adamson, A. S., et al., JAMA Dermatol, 2017. 153(11): p. 1106-1113).

As sophisticated hospital-based imaging procedures will only increase in price over time there is a need for novel affordable outpatient surveillance solutions such as those described in this application. Indeed, to lessen the heavy toll of melanoma, effective screening approaches are urgently needed. There is an imperative for innovation that will deliver accurate, affordable, and safe screening tools for the pre-symptomatic detection of earliest stage of melanoma and advanced melanoma.

The present invention addresses such needs. Indeed, the present invention provides novel methylated DNA markers that discriminate cases of melanoma and its various subtypes (e.g., metastatic melanoma, primary melanoma) within various biological samples (e.g., tissue, blood).

### SUMMARY

Blood tests for melanoma screening have typically been based on marker detection in the clear portion of blood (plasma or serum) and have been insensitive or nonspecific for earliest stage disease. To address these historical deficiencies, experiments conducted herein explored a rational new approach anchored on discriminant methylated DNA markers. Marker discovery and validation occurred through utilization of high quality flash-frozen bio-banked tissues. Feasibility of marker detection in blood compartments was established by use of an exquisitely sensitive analytical platform (e.g., a quantitative methylation-specific PCR; qMSP). Such results indicate that this blood testing approach establishes a requisite analytical sensitivity to detect curable-stage cancers while accurately predicting tumor site and avoiding frequent false positives.

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression. In a biologically attractive mechanism, acquired methylation events in promoter regions of tumor suppressor genes are thought to silence expression, thus contributing to oncogenesis. DNA methylation may be a more chemically and biologically stable diagnostic tool than RNA or protein expression (Laird (2010) Nat Rev Genet 11: 191-203). Furthermore, in other cancers like sporadic colon cancer, methylation markers offer excellent specificity and are more broadly informative and sensitive than individual DNA mutations (Zou et al (2007) Cancer Epidemiol Biomarkers Prev 16: 2686-96).

Analysis of CpG islands has yielded important findings when applied to animal models and human cell lines. For example, Zhang and colleagues found that amplicons from different parts of the same CpG island may have different levels of methylation (Zhang et al. (2009) PLoS Genet 5: e1000438). Further, methylation levels were distributed bi-modally between highly methylated and unmethylated sequences, further supporting the binary switch-like pattern of DNA methyltransferase activity (Zhang et al. (2009) PLoS Genet 5: e1000438). Analysis of murine tissues in vivo and cell lines in vitro demonstrated that only about 0.3% of high CpG density promoters (HCP, defined as having >7% CpG sequence within a 300 base pair region) were methylated, whereas areas of low CpG density (LCP, defined as having <5% CpG sequence within a 300 base pair region) tended to be frequently methylated in a dynamic tissue-specific pattern (Meissner et al. (2008) Nature 454: 766-70). HCPs include promoters for ubiquitous housekeeping genes and highly regulated developmental genes. Among the HCP sites methylated at >50% were several established markers such as Wnt 2, NDRG2, SFRP2, and BMP3 (Meissner et al. (2008) Nature 454: 766-70).

Epigenetic methylation of DNA at cytosine-phosphate-guanine (CpG) island sites by DNA methyltransferases has been studied as a potential class of biomarkers in the tissues of most tumor types.

Several methods are available to search for novel methylation markers. While micro-array-based interrogation of CpG methylation is a reasonable, high-throughput approach, this strategy is biased towards known regions of interest, mainly established tumor suppressor promotors. Alternative methods for genome-wide analysis of DNA methylation have been developed in the last decade. There are four basic approaches. The first employs digestion of DNA by restriction enzymes which recognize specific methylated sites, followed by several possible analytic techniques which provide methylation data limited to the enzyme recognition site or the primers used to amplify the DNA in quantification steps (such as methylation-specific PCR; MSP). A second approach enriches methylated fractions of genomic DNA using anti-bodies directed to methyl-cytosine or other methylation-specific binding domains followed by microarray analysis or sequencing to map the fragment to a reference genome. This approach does not provide single nucleotide resolution of all methylated sites within the fragment. A third approach begins with bisulfite treatment of the DNA to convert all unmethylated cytosines to uracil, followed by restriction enzyme digestion and complete sequencing of all fragments after coupling to an adapter ligand. The choice of restriction enzymes can enrich the fragments for CpG dense regions, reducing the number of redundant sequences which may map to multiple gene positions during analysis. A fourth approach involves a bisulfite-free treatment of the DNA that describe a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines (see, Liu et al., 2019, Nat Biotechnol. 37, pp. 424-429). In some embodiments, regardless of the specific enzymatic conversion approach, only the methylated cytosines are converted.

Reduced Representation Bisulfite Sequencing (RRBS) yields CpG methylation status data at single nucleotide resolution of 80-90% of all CpG islands and a majority of tumor suppressor promoters at medium to high read coverage. In cancer case—control studies, analysis of these reads results in the identification of differentially methylated regions (DMRs). In previous RRBS analysis of pancreatic cancer specimens, hundreds of DMRs were uncovered, many of which had never been associated with carcinogenesis and many of which were unannotated. Further validation studies on independent tissue sample sets confirmed marker CpGs which were 100% sensitive and specific in terms of performance.

Provided herein is technology for melanoma and various melanoma subtypes (e.g., metastatic melanoma, primary cutaneous melanoma) screening and particularly, but not exclusively, to methods, compositions, and related uses for detecting the presence of melanoma and various melanoma subtypes (e.g., metastatic melanoma, primary cutaneous melanoma).

Indeed, as described in Examples I and II, experiments conducted during the course for identifying embodiments for the present invention identified a novel set of differentially methylated regions (DMRs) for discriminating 1) melanoma derived DNA from non-neoplastic control tissue, 2) DNA derived from metastatic melanoma tissue from non-neoplastic control DNA, and 3) DNA derived from primary cutaneous melanoma tissue from non-neoplastic control DNA.

Such experiments list and describe 331 novel DNA methylation markers distinguishing melanoma tissue from benign tissue (see, Tables 1A, 1B, 4, 5A, 5B, 5C, 7A, and 7B; Examples I and II), metastatic melanoma tissue from benign tissue (see, Tables 5C; Example I), primary cutaneous melanoma tissue from benign tissue (see, Tables 5C; Example I), and detecting melanoma within a blood sample (see, Tables 2A, 2B, 4, 5A, 6 and 8A; Examples I and II).

From these 331 novel DNA methylation markers, further experiments identified the following markers and/or panels of markers capable of distinguishing melanoma tissue from benign tissue:
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Table 4, 5A, 5B; Example I);
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Table 7A and 7B; Example II);
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10, 11; Example II).

From these 331 novel DNA methylation markers, further experiments identified the following markers and/or panels of markers capable of distinguishing metastatic melanoma tissue from benign tissue:
MAX.chr20.21080958-21081038, MAX.chr7.155259597-155259763, FOXL2NB, and HOXA9_A (see, Table 5C, Example I); and
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10, 11; Example II).

From these 331 novel DNA methylation markers, further experiments identified the following markers and/or panels of markers capable of distinguishing primary cutaneous melanoma tissue from benign tissue:
MAX.chr20.21080958-21081038, MAX.chr7.155259597-155259763, FOXL2NB, and HOXA9_A (see, Table 5C, Example I);
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10, 11; Example II).

From these 331 novel DNA methylation markers, further experiments identified the following markers and/or panels of markers for detecting melanoma in blood samples (e.g., plasma samples, whole blood samples, leukocyte samples, serum samples):
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Table 4, 5A, 5B, 6; Example I);
one or more of the markers recited in Tables 8A and 8B; and
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10, 11; Example II).

As described herein, the technology provides a number of methylated DNA markers and subsets thereof (e.g., sets of 2, 3, 4, 5, 6, 7, or 8 markers) with high discrimination for melanoma overall and various types of melanoma (e.g., metastatic melanoma, primary cutaneous melanoma). Experiments applied a selection filter to candidate markers to identify markers that provide a high signal to noise ratio and a low background level to provide high specificity for purposes of melanoma screening or diagnosis.

In some embodiments, the technology is related to assessing the presence of and methylation state of one or more of the markers identified herein in a biological sample (e.g., melanoma tissue, plasma sample). These markers comprise one or more differentially methylated regions (DMR) as discussed herein, e.g., as provided in Tables 1A, 2A, 7A, 8A, and 9. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-sensitive arbitrarily primed PCR (Gonzalgo et al. (1997) Cancer Res. 57: 594-599). In some embodiments, changes in methylation patterns at specific CpG sites are monitored by digestion of genomic DNA with methylation-sensitive restriction enzymes followed by Southern analysis of the regions of interest (digestion-Southern method). In some embodiments, analyzing changes in methylation patterns involves a PCR-based process that involves digestion of genomic DNA with methylation-sensitive restriction enzymes or methylation-dependent restriction enzymes prior to PCR amplification (Singer-Sam et al. (1990) Nucl. Acids Res. 18: 687). In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated (or non-bisulfite treated (see, Liu et al., 2019, Nat Biotechnol. 37, pp. 424-429)) and the resulting methylation-specific sequence is determined using either a real-time PCR or other exponential amplification, e.g., a QuARTS assay (e.g., as provided by U.S. Pat. Nos. 8,361,720; 8,715,937; 8,916,344; and 9,212,392).

For example, in some embodiments. methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amount of methylated DNAs relative to the total amount of DNAs in the population, e.g., (number of methylated DNAs)/(the number of methylated DNAs+number of unmethylated DNAs)×100.

In some embodiments, the plurality of different target regions comprise a reference target region, and in certain preferred embodiments, the reference target region comprises 3-actin and/or ZDHHC1, and/or B3GALT6.

Also provided herein are compositions and kits for practicing the methods. For example, in some embodiments, reagents (e.g., primers, probes) specific for one or more MDMs are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane, or other assays). In some embodiments, the kits contain a reagent capable of modifying DNA in a methylation-specific manner (e.g., a methylation-sensitive restriction enzyme, a methylation-dependent restriction enzyme, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane), and/or an agent capable of detecting an increased level of a protein marker described herein. In some embodiments, the kits containing one or more reagents necessary, sufficient, or useful for conducting a method are provided. Also provided are reaction mixtures containing the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMR, e.g., DMR 1-331 as provided in Tables 1A, 2A, 7A, 8A, and 9); comparing methylation states (e.g., of one or more DMR, e.g., DMR 1-331 as provided in Tables 1A, 2A, 7A, 8A, and 9); generating standard curves; determining a Ct value; calculating a fraction, frequency, or percentage of methylation (e.g., of one or more DMR, e.g., DMR 1-331 as provided in Tables 1A, 2A, 7A, 8A, and 9); identifying a CpG island; determining a specificity and/or sensitivity of an assay or marker; calculating an ROC curve and an associated AUC; sequence analysis; all as described herein or is known in the art.

In some embodiments, a microprocessor or computer uses methylation state data in an algorithm to predict a site of a cancer.

In some embodiments, a software or hardware component receives the results of multiple assays and determines a single value result to report to a user that indicates a cancer risk based on the results of the multiple assays (e.g., determining the methylation state of multiple DMR, e.g., as provided in Tables 1A, 2A, 7A, 8A, and 9). Related embodiments calculate a risk factor based on a mathematical combination (e.g., a weighted combination, a linear combination) of the results from multiple assays, e.g., determining the methylation states of multiple markers (such as multiple DMR, e.g., as provided in Tables 1A, 2A, 7A, 8A, and 9). In some embodiments, the methylation state of a DMR defines a dimension and may have values in a multidimensional space and the coordinate defined by the methylation states of multiple DMR is a result, e.g., to report to a user, e.g., related to a cancer risk.

Some embodiments comprise a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

Accordingly, provided herein is technology related to a method of screening for melanoma and/or various forms of melanoma (e.g., metastatic melanoma, primary cutaneous melanoma) in a sample obtained from a subject, the method comprising assaying a methylation state of a marker in a sample obtained from a subject (e.g., melanoma tissue) (e.g., tissue sample, plasma sample) and identifying the subject as having melanoma and/or various forms of melanoma (e.g., metastatic melanoma, primary cutaneous melanoma) when the methylation state of the marker is different than a methylation state of the marker assayed in a subject that does not have such cancer, wherein the marker comprises a base in a differentially methylated region (DMR) selected from a group consisting of DMR 1-331 as provided in Tables 1A, 2A, 7A, 8A, and 9.

In some embodiments wherein the sample obtained from the subject is skin tissue and the methylation state of one or more of the following markers is different than a methylation state of the one or more markers assayed in a subject that does not have melanoma indicates the subject has melanoma: c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, and 5B).

In some embodiments wherein the sample obtained from the subject is skin tissue and the methylation state of one or more of the following markers is different than a methylation state of the one or more markers assayed in a subject that does not have melanoma indicates the subject has melanoma: MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

In some embodiments wherein the sample obtained from the subject is skin tissue and the methylation state of one or more of the following markers is different than a methylation state of the one or more markers assayed in a subject that does not have melanoma indicates the subject has melanoma: AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B).

In some embodiments wherein the sample obtained from the subject is skin tissue and the methylation state of one or more of the following markers is different than a methylation state of the one or more markers assayed in a subject that does not have metastatic melanoma indicates the subject has metastatic melanoma: MAX.chr20.21080958-21081038, MAX.chr7.155259597-155259763, FOXL2NB, and HOXA9_A (see, Table 5C, Example I).

In some embodiments wherein the sample obtained from the subject is skin tissue and the methylation state of one or more of the following markers is different than a methylation state of the one or more markers assayed in a subject that does not have metastatic melanoma indicates the subject has metastatic melanoma: MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

In some embodiments wherein the sample obtained from the subject is skin tissue and the methylation state of one or more of the following markers is different than a methylation state of the one or more markers assayed in a subject that does not have primary cutaneous melanoma indicates the subject has primary cutaneous melanoma: MAX.chr20.21080958-21081038, MAX.chr7.155259597-155259763, FOXL2NB, and HOXA9_A (see, Table 5C, Example I).

In some embodiments wherein the sample obtained from the subject is skin tissue and the methylation state of one or more of the following markers is different than a methylation state of the one or more markers assayed in a subject that does not have primary cutaneous melanoma indicates the subject has primary cutaneous melanoma: MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have melanoma indicates the subject has melanoma: c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Table 4, 5A, 5B, 6; Example I).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have melanoma indicates the subject has melanoma: MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have melanoma indicates the subject has melanoma: one or more of the markers recited in Table 8A.

The technology is related to identifying and discriminating melanoma and/or various forms of melanoma (e.g., metastatic melanoma, primary cutaneous melanoma). Some embodiments provide methods comprising assaying a plurality of markers, e.g., comprising assaying 1, 2, 3, 2 to 11 to 100 or 120 or 331 markers (e.g., 1-4, 1-6, 1-7, 1-8, 1-9, 1-10, 1-11, 1-12, 1-13, 1-14, 1-15, 1-16, 1-17, 1-18, 1-19, 1-20, 1-25, 1-50, 1-75, 1-100, 1-200, 1-300, 1-331) (e.g., 2-4, 2-6, 2-7, 2-8, 2-9, 2-10, 2-11, 2-12, 2-13, 2-14, 2-15, 2-16, 2-17, 2-18, 2-19, 2-20, 2-25, 2-50, 2-75, 2-100, 2-200, 2-300, 2-331) (e.g., 3-4, 3-6, 3-7, 3-8, 3-9, 3-10, 3-11, 3-12, 3-13, 3-14, 3-15, 3-16, 3-17, 3-18, 3-19, 3-20, 3-25, 3-50, 3-75, 3-100, 3-200, 3-300, 3-331) (e.g., 4-5, 4-6, 4-7, 4-8, 4-9, 4-10, 4-11, 4-12, 4-13, 4-14, 4-15, 4-16, 4-17, 4-18, 4-19, 4-20, 4-25, 4-50, 4-75, 4-100, 4-200, 4-300, 4-331) (e.g., 5-6, 5-7, 5-8, 5-9, 5-10, 5-11, 5-12, 5-13, 5-14, 5-15, 5-16, 5-17, 5-18, 5-19, 5-20, 5-25, 5-50, 5-75, 5-100, 5-200, 5-300, 5-331).

The technology is not limited in the methylation state assessed. In some embodiments assessing the methylation state of the marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample (e.g., skin tissue sample), lymphatic tissue, deep tissue biopsy, a blood sample (e.g., plasma, serum, whole blood), an excretion, or a urine sample. In some embodiments, the sample is a fine needle aspirate. In some embodiments, the sample is taken by using a sampling device such as a swab or tape with adhesive to collect cells on the skin surface. Malignant melanoma is characterized by spread to other organs and deep tissue types, especially to lymph nodes, and to other organs such as the lungs, liver, bone or brain.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

The technology provides reagents for detecting a DMR, e.g., in some embodiments are provided a set of oligonucleotides comprising the sequences provided by SEQ ID NO: 1-167 (see, Table 3, 10 and 11). In some embodiments are provided an oligonucleotide comprising a sequence complementary to a chromosomal region having a base in a DMR, e.g., an oligonucleotide sensitive to methylation state of a DMR.

The technology provides various panels of markers use for identifying melanoma, e.g., in some embodiments the marker comprises a chromosomal region having an annotation that is c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, 5B).

The technology provides various panels of markers use for identifying melanoma, e.g., in some embodiments the marker comprises a chromosomal region having an annotation that is MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

The technology provides various panels of markers use for identifying melanoma, e.g., in some embodiments the marker comprises a chromosomal region having an annotation that is AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B).

The technology provides various panels of markers use for identifying metastatic melanoma, e.g., in some embodiments the marker comprises a chromosomal region having an annotation that is MAX.chr20.21080958-21081038, MAX.chr7.155259597-155259763, FOXL2NB, and HOXA9_A (see, Table 5C, Example I).

The technology provides various panels of markers use for identifying metastatic melanoma, e.g., in some embodiments the marker comprises a chromosomal region having an annotation that is MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

The technology provides various panels of markers use for identifying primary cutaneous melanoma, e.g., in some embodiments the marker comprises a chromosomal region having an annotation that is MAX.chr20.21080958-21081038, MAX.chr7.155259597-155259763, FOXL2NB, and HOXA9_A (see, Table 5C, Example I).

The technology provides various panels of markers use for identifying primary cutaneous melanoma, e.g., in some embodiments the marker comprises a chromosomal region having an annotation that is MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11).

The technology provides various panels of markers use for identifying melanoma, e.g., in some embodiments the marker comprises a chromosomal region having an annotation that is recited in Table 8A (see, Example II).

Kit embodiments are provided, e.g., a kit comprising a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and a control nucleic acid comprising one or more sequences from DMR 1-331 (from Tables 1A, 2A, 7A, 8A, and 9) and having a methylation state associated with a subject who does not have cancer. In some embodiments, kits comprise a bisulfite reagent and an oligonucleotide as described herein. In some embodiments, kits comprise a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and a control nucleic acid comprising one or more sequences from DMR 1-331 (from Tables 1A, 2A, 7A, 8A, and 9) and having a methylation state associated with a subject who has a specific type of cancer. Some kit embodiments comprise a sample collector for obtaining a sample from a subject (e.g., a stool sample; tissue sample; plasma sample, serum sample, whole blood sample); a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and an oligonucleotide as described herein.

The technology is related to embodiments of compositions (e.g., reaction mixtures). In some embodiments are provided a composition comprising a nucleic acid comprising a DMR and a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

Additional related method embodiments are provided for screening for melanoma and/or various forms of melanoma (e.g., metastatic melanoma, primary cutaneous melanoma) in a sample obtained from a subject (e.g., a stool sample; skin tissue sample; fine needle aspirate, deep tissue sample, plasma sample, serum sample, whole blood sample), e.g., a method comprising determining a methylation state of a marker in the sample comprising a base in a DMR that is one or more of DMR 1-331 (from Tables 1A, 2A, 7A, 8A, and 9); comparing the methylation state of the marker from the subject sample to a methylation state of the marker from a normal control sample from a subject who does not have melanoma (e.g., melanoma and/or a form of melanoma: metastatic melanoma, primary cutaneous melanoma); and determining a confidence interval and/or a p value of the difference in the methylation state of the subject sample and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a DMR with a reagent capable of modifying nucleic acid in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane) to produce, for example, nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have a specific type of cancer to identify differences in the two sequences; and identifying the subject as having melanoma and/or a form of melanoma (e.g., metastatic melanoma, primary cutaneous melanoma) when a difference is present.

Systems for screening for melanoma in a sample obtained from a subject are provided by the technology. Exemplary embodiments of systems include, e.g., a system for screening for melanoma and/or types of melanoma (e.g., metastatic melanoma, primary cutaneous melanoma) in a sample obtained from a subject (e.g., a stool sample; skin tissue sample; fine needle aspirate, deep tissue sample, plasma sample, serum sample, whole blood sample), the system comprising an analysis component configured to determine the methylation state of a sample, a software component configured to compare the methylation state of the sample with a control sample or a reference sample methylation state recorded in a database, and an alert component configured to alert a user of a melanoma-associated methylation state. An alert is determined in some embodiments by a software component that receives the results from multiple assays (e.g., determining the methylation states of multiple markers, e.g., DMR, e.g., as provided in Tables 1A, 2A, 7A, 8A, and 9) and calculating a value or result to report based on the multiple results. Some embodiments provide a database of weighted parameters associated with each DMR provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays that is indicative of a cancer risk in a subject.

In some embodiments of systems, a sample comprises a nucleic acid comprising a DMR. In some embodiments the system further comprises a component for isolating a nucleic acid, a component for collecting a sample such as a component for collecting a stool sample. In some embodiments, the system comprises nucleic acid sequences comprising a DMR. In some embodiments the database comprises nucleic acid sequences from subjects who do not have melanoma and/or specific types of melanoma (e.g., metastatic melanoma, primary cutaneous melanoma). Also provided are nucleic acids, e.g., a set of nucleic acids, each nucleic acid having a sequence comprising a DMR. In some embodiments the set of nucleic acids wherein each nucleic acid has a sequence from a subject who does not have melanoma and/or specific types of melanoma. Related system embodiments comprise a set of nucleic acids as described and a database of nucleic acid sequences associated with the set of nucleic acids. Some embodiments further comprise a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane). Some embodiments further comprise a nucleic acid sequencer.

In certain embodiments, methods for characterizing a sample (e.g., a stool sample; skin tissue sample; fine needle aspirate, deep tissue sample, plasma sample, serum sample, whole blood sample) from a human patient are provided. For example, in some embodiments such embodiments comprise obtaining DNA from a sample of a human patient; assaying a methylation state of a DNA methylation marker comprising a base in a differentially methylated region (DMR) selected from a group consisting of DMR 1-331 from Tables TA, 2A, 7A, 8A, and 9; and comparing the assayed methylation state of the one or more DNA methylation markers with methylation level references for the one or more DNA methylation markers for human patients not having melanoma and/or specific types of melanoma (e.g., metastatic melanoma, primary cutaneous melanoma).

Such methods are not limited to a particular type of sample from a human patient. In some embodiments, the sample is a skin tissue sample. In some embodiments, the sample is a plasma sample. In some embodiments, the sample is a stool sample, a tissue sample, a skin tissue sample, a blood sample (e.g., a stool sample; skin tissue sample; fine needle aspirate, deep tissue sample, plasma sample, serum sample, whole blood sample).

In some embodiments, such methods comprise assaying a plurality of DNA methylation markers (e.g., 1-4, 1-6, 1-7, 1-8, 1-9, 1-10, 1-11, 1-12, 1-13, 1-14, 1-15, 1-16, 1-17, 1-18, 1-19, 1-20, 1-25, 1-50, 1-75, 1-100, 1-200, 1-300, 1-331) (e.g., 2-4, 2-6, 2-7, 2-8, 2-9, 2-10, 2-11, 2-12, 2-13, 2-14, 2-15, 2-16, 2-17, 2-18, 2-19, 2-20, 2-25, 2-50, 2-75, 2-100, 2-200, 2-300, 2-331) (e.g., 3-4, 3-6, 3-7, 3-8, 3-9, 3-10, 3-11, 3-12, 3-13, 3-14, 3-15, 3-16, 3-17, 3-18, 3-19, 3-20, 3-25, 3-50, 3-75, 3-100, 3-200, 3-300, 3-331) (e.g., 4-5, 4-6, 4-7, 4-8, 4-9, 4-10, 4-11, 4-12, 4-13, 4-14, 4-15, 4-16, 4-17, 4-18, 4-19, 4-20, 4-25, 4-50, 4-75, 4-100, 4-200, 4-300, 4-331) (e.g., 5-6, 5-7, 5-8, 5-9, 5-10, 5-11, 5-12, 5-13, 5-14, 5-15, 5-16, 5-17, 5-18, 5-19, 5-20, 5-25, 5-50, 5-75, 5-100, 5-200, 5-300, 5-331). In some embodiments, such methods comprise assaying 2 to 11 DNA methylation markers. In some embodiments, such methods comprise assaying 12 to 120 DNA methylation markers. In some embodiments, such methods comprise assaying 2 to 331 DNA methylation markers. In some embodiments, such methods comprise assaying the methylation state of the one or more DNA methylation markers in the sample comprises determining the methylation state of one base. In some embodiments, such methods comprise assaying the methylation state of the one or more DNA methylation markers in the sample comprises determining the extent of methylation at a plurality of bases. In some embodiments, such methods comprise assaying a methylation state of a forward strand or assaying a methylation state of a reverse strand.

In some embodiments, the DNA methylation marker is a region of 100 or fewer bases. In some embodiments, the DNA methylation marker is a region of 500 or fewer bases. In some embodiments, the DNA methylation marker is a region of 1000 or fewer bases. In some embodiments, the DNA methylation marker is a region of 5000 or fewer bases. In some embodiments, the DNA methylation marker is one base. In some embodiments, the DNA methylation marker is in a high CpG density promoter.

In some embodiments, the assaying comprises using methylation specific polymerase chain reaction, nucleic acid sequencing, mass spectrometry, methylation specific nuclease, mass-based separation, or target capture.

In some embodiments, the assaying comprises use of a methylation specific oligonucleotide. In some embodiments, the methylation specific oligonucleotide is selected from the group consisting of SEQ ID NO: 1-80 (Table 3).

In some embodiments, a chromosomal region having an annotation selected from the group consisting of c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, 5B and 6) comprises the DNA methylation marker.

In some embodiments, a chromosomal region having an annotation selected from the group consisting of AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B) comprises the DNA methylation marker.

In some embodiments, a chromosomal region having an annotation selected from the group consisting of MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11) comprises the DNA methylation marker.

In some embodiments, a chromosomal region having an annotation recited in Table 8A (see, Example II) comprises the DNA methylation marker.

In some embodiments, a chromosomal region having an annotation selected from the group consisting of MAX.chr20.21080958-21081038, MAX.chr7.155259597-155259763, FOXL2NB, and HOXA9_A (see, Table 5C, Example I) comprises the DNA methylation marker.

In some embodiments, such methods comprise determining the methylation state of two DNA methylation markers. In some embodiments, such methods comprise determining the methylation state of a pair of DNA methylation markers provided in Tables 1A, 2A, 7A 8A, and/or 9.

In certain embodiments, the technology provides methods for characterizing a sample (e.g., a stool sample; skin tissue sample; fine needle aspirate, deep tissue sample, plasma sample, serum sample, whole blood sample) obtained from a human patient. In some embodiments, such methods comprise determining a methylation state of a DNA methylation marker in the sample comprising a base in a DMR selected from a group consisting of DMR 1-331 from Tables 1A, 2A, 7A, 8A, and 9; comparing the methylation state of the DNA methylation marker from the patient sample to a methylation state of the DNA methylation marker from a normal control sample from a human subject who does not have a melanoma and/or a specific form of melanoma (e.g., metastatic melanoma, primary cutaneous melanoma); and determining a confidence interval and/or a p value of the difference in the methylation state of the human patient and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001.

In certain embodiments, the technology provides methods for characterizing a sample obtained from a human subject (e.g., a stool sample; skin tissue sample; fine needle aspirate, deep tissue sample, plasma sample, serum sample, whole blood sample), the method comprising reacting a nucleic acid comprising a DMR with a reagent capable of modifying DNA in a methylation-specific manner (e.g., a methylation-sensitive restriction enzyme, a methylation-dependent restriction enzyme, and a bisulfite reagent) to produce nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have melanoma to identify differences in the two sequences.

In certain embodiments, the technology provides systems for characterizing a sample obtained from a human subject (e.g., a stool sample; skin tissue sample; fine needle aspirate, deep tissue sample, plasma sample, serum sample, whole blood sample), the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a melanoma-associated methylation state. In some embodiments, the sample comprises a nucleic acid comprising a DMR.

In some embodiments, such systems further comprise a component for isolating a nucleic acid. In some embodiments, such systems further comprise a component for collecting a sample.

In some embodiments, the sample is a stool sample, a tissue sample, a skin tissue sample, fine needle aspirate, a deep tissue sample, a lymph node sample, a blood sample (e.g., plasma sample, whole blood sample, serum sample), or a urine sample.

In some embodiments, the database comprises nucleic acid sequences comprising a DMR. In some embodiments, the database comprises nucleic acid sequences from subjects who do not have a melanoma.

In some embodiments, the sample is a stool sample, a tissue sample (e.g., skin tissue), a blood sample (e.g., plasma sample, whole blood sample, serum sample), or a urine sample. In some embodiments, the sample comprises blood, serum, plasma, gastric secretions, pancreatic juice, a cerebral spinal fluid (CSF) sample, a gastrointestinal biopsy sample, and/or cells recovered from stool. In some embodiments, the subject is human. The sample may include cells, secretions, or tissues from the lymph gland, breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, gastric section, pancreatic fluid, fluid obtained during endoscopy, blood.

Additional embodiments will be apparent to persons skilled in the relevant art based on the teachings contained herein.

### BRIEF DESCRIPTION OF THE DRAWINGS

FIG. 1: Marker chromosomal regions used for the methylated DNA markers recited in Table 9 and related primer and probe information (Tables 10 and 11).

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and poly adenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-beta replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics 4:560). Finally, thermostable template-dependant DNA polymerases (e.g., Taq and Pfu DNA polymerases), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction. As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., β-actin, ZDHHC1, and B3GALT6 (see, e.g., U.S. patent application Ser. Nos 14/966,617 and 62/364,082, each incorporated herein by reference.

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In particularly preferred embodiments, plasmid calibrators are digested, e.g., with one or more restriction enzymes, to release calibrator portion from the plasmid vector. See, e.g., WO 2015/066695, which is included herein by reference.

As used herein “ZDHHC1” refers to a gene encoding a protein characterized as a zinc finger, DHHC-type containing 1, located in human DNA on Chr 16 (16q22.1) and belonging to the DHHC palmitoyltransferase family. As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

In some embodiments, the sample is a stool sample, a tissue sample (e.g., skin tissue), a blood sample (e.g., plasma sample, whole blood sample, serum sample), or a urine sample. In some embodiments, the sample comprises blood, serum, plasma, gastric secretions, pancreatic juice, a cerebral spinal fluid (CSF) sample, a gastrointestinal biopsy sample, and/or cells recovered from stool. In some embodiments, the subject is human. The sample may include cells, secretions, or tissues from the lymph gland, breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, gastric section, pancreatic fluid, fluid obtained during endoscopy, blood.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, and a bisulfite reagent.

A change in the nucleic acid nucleotide sequence by a methylation-specific reagent can also result in a nucleic acid molecule in which each methylated nucleotide is modified to a different nucleotide.

As used herein, the term “UDP glucose modified with a chemoselective group” refers to a uridine diphosphoglucose molecule that has been functionalized, particularly at the 6-hydroxyl position, with a functional group capable of reaction with an affinity tag via click chemistry.

The term “oxidized 5-methylcytosine” refers to an oxidized 5-methylcytosine residue that has been oxidized at the 5-position. Oxidized 5-methylcytosine residues thus include 5-hydroxymethylcytosine, 5-formylcytosine, and 5-carboxymethylcytosine. The oxidized 5-methylcytosine residues that undergo reaction with an organic borane according to one embodiment of the invention are 5-formylcytosine and 5-carboxymethylcytosine.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Nat. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, a “remote sample” as used in some contexts relates to a sample collected from a site that is not the cell, tissue, or organ source of the sample.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including race horses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “nevi” or “mole” refers to a typically noncancerous skin growth made up of cells (melanocytes or nevus cells) that produce color (pigment). Moles can appear anywhere on the skin, alone or in groups.

As used herein, the term “lesion” refers to a mole that is under examination (e.g., is suspected of being cancerous or has been diagnosed as cancerous) and may or may not be cancerous. In some embodiments, “lesion” is used interchangeably with “mole” or “nevi.”

As used herein, the term “melanoma” or “malignant melanoma” refers to a serious form of skin cancer that may affect the skin only or may spread (metastasize) through the blood or lymph systems to organs and bones. Melanoma can develop in an existing mole or other mark on the skin or on unmarked skin.

As used herein, the term “metastatic melanoma” refers to melanoma that has spread to other tissues or organs.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DETAILED DESCRIPTION

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

Provided herein is technology for melanoma screening and particularly, but not exclusively, to methods, compositions, and related uses for detecting the presence of melanoma and/or specific forms of melanoma (e.g., metastatic melanoma, primary cutaneous melanoma). As the technology is described herein, the section headings used are for organizational purposes only and are not to be construed as limiting the subject matter in any way.

Indeed, as described in Examples I and II, experiments conducted during the course for identifying embodiments for the present invention identified a novel set of 331 differentially methylated regions (DMRs) for discriminating melanoma derived DNA from non-neoplastic control DNA. From these 331 novel DNA methylation markers, further experiments identified markers capable of distinguishing different types of melanoma from normal tissue. For example, separate sets of DMRs were identified capable of distinguishing 1) metastatic melanoma tissue from normal tissue, and 2) primary cutaneous melanoma tissue from normal tissue.

Although the disclosure herein refers to certain illustrated embodiments, it is to be understood that these embodiments are presented by way of example and not by way of limitation.

In particular aspects, the present technology provides compositions and methods for identifying, determining, and/or classifying a cancer such as melanoma and/or a sub-type of melanoma (e.g., metastatic melanoma, primary cutaneous melanoma). The methods comprise determining the methylation status of at least one methylation marker in a biological sample isolated from a subject (e.g., a stool sample; skin tissue sample; fine needle aspirate, deep tissue sample, plasma sample, serum sample, whole blood sample), wherein a change in the methylation state of the marker is indicative of the presence, class, or site of melanoma and/or a sub-type thereof. Particular embodiments relate to markers comprising a differentially methylated region (DMR, e.g., DMR 1-331, see Tables 1A, 2A, 7A, 8A, and 9) that are used for diagnosis (e.g., screening) of melanoma and various types of melanoma (e.g., metastatic melanoma, primary cutaneous melanoma).

In addition to embodiments wherein the methylation analysis of at least one marker, a region of a marker, or a base of a marker comprising a DMR (e.g., DMR 1-331) provided herein and listed in Tables 1A, 2A, 7A, 8A, and 9 is analyzed, the technology also provides panels of markers comprising at least one marker, region of a marker, or base of a marker comprising a DMR with utility for the detection of cancers, in particular melanoma.

Some embodiments of the technology are based upon the analysis of the CpG methylation status of at least one marker, region of a marker, or base of a marker comprising a DMR.

In some embodiments, the present technology provides for the use of a reagent that modifies DNA in a methylation-specific manner (e.g., a methylation-sensitive restriction enzyme, a methylation-dependent restriction enzyme, and a bisulfite reagent) in combination with one or more methylation assays to determine the methylation status of CpG dinucleotide sequences within at least one marker comprising a DMR (e.g., DMR 1-331, see Tables 1A, 2A, 7A, 8A, and 9). Genomic CpG dinucleotides can be methylated or unmethylated (alternatively known as up- and down-methylated respectively). However, the methods of the present invention are suitable for the analysis of biological samples of a heterogeneous nature, e.g., a low concentration of tumor cells, or biological materials therefrom, within a background of a remote sample (e.g., blood, organ effluent, or stool). Accordingly, when analyzing the methylation status of a CpG position within such a sample one may use a quantitative assay for determining the level (e.g., percent, fraction, ratio, proportion, or degree) of methylation at a particular CpG position.

According to the present technology, determination of the methylation status of CpG dinucleotide sequences in markers comprising a DMR has utility both in the diagnosis and characterization of cancers such as melanoma.

### Combinations of Markers

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392).

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components. Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides according to the present invention (e.g., see Table V) and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In some embodiments, the technology relates to assessing the methylation state of combinations of markers comprising a DMR from Tables 1A, 2A, 7A, 8A, and 9 (e.g., DMR Nos. 1-331). In some embodiments, assessing the methylation state of more than one marker increases the specificity and/or sensitivity of a screen or diagnostic for identifying a neoplasm in a subject (e.g., melanoma).

In another embodiment, the invention provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with an organic borane. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5 mC or 5hmC, e.g., with potassium perruthenate (KRuO₄) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The organic borane may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine.

The reaction of the organic borane with the oxidized 5mC residue in cell-free DNA is advantageous insofar as non-toxic reagents and mild reaction conditions can be employed; there is no need for any bisulfite, nor for any other potentially DNA-degrading reagents. Furthermore, conversion of an oxidized 5mC residue to dihydrouracil with the organic borane can be carried out without need for isolation of any intermediates, in a “one-pot” or “one-tube” reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the invention also provides a reaction mixture related to the aforementioned method. The reaction mixture comprises a sample of cell-free DNA containing at least one oxidized 5-methylcytosine residue selected from 5caC, 5fC, and combinations thereof, and an organic borane effective to reduce, deaminate, and either decarboxylate or deformylate the at least one oxidized 5-methylcytosine residue. The organic borane is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the invention, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and an organic borane effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps:

(a) modifying 5hmC residues in fragmented, adapter-ligated cell-free DNA to provide an affinity tag thereon, wherein the affinity tag enables removal of modified 5hmC-containing DNA from the cell-free DNA;

(b) removing the modified 5hmC-containing DNA from the cell-free DNA, leaving DNA containing unmodified 5mC residues;

(c) oxidizing the unmodified 5mC residues to give DNA containing oxidized 5mC residues selected from 5caC, 5fC, and combinations thereof;

(d) contacting the DNA containing oxidized 5mC residues with an organic borane effective to reduce, deaminate, and either decarboxylate or deformylate the oxidized 5mC residues, thereby providing DNA containing dihydrouracil residues in place of the oxidized 5mC residues;

(e) amplifying and sequencing the DNA containing dihydrouracil residues;

(f) determining a 5-methylation pattern from the sequencing results in (e).

The cell-free DNA is extracted from a body sample from a subject, where the body sample is typically whole blood, plasma, or serum, most typically plasma, but the sample may also be urine, saliva, mucosal excretions, sputum, stool, or tears. In some embodiments, the cell-free DNA is derived from a tumor. In other embodiments, the cell-free DNA is from a patient with a disease or other pathogenic condition. The cell-free DNA may or may not derive from a tumor. In step (a), it should be noted that the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In step (a), then, the “purified, fragmented cell-free DNA” comprises adapter-ligated DNA fragments. Modification of 5hmC residues in these cell-free DNA fragments with an affinity tag, as specified in step (a), is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin, e.g., streptavidin beads, magnetic streptavidin beads, etc.

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down in step (b) using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In step (c), the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, step (e) converts both 5fC residues and 5caC residues to dihydrouracil (DHU).

In some embodiments, 5-hydroxymethylcytosine residues are blocked with (3-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another organic borane to give dihydrouracil. In a variation on this embodiment, 5hmC-containing fragments are not removed in step (b). Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

There are numerous advantages to the method of this embodiment: bisulfite is unnecessary, nontoxic reagents and reactants are employed; and the process proceeds under mild conditions. In addition, the entire process can be performed in a single tube, without need for isolation of any intermediates.

In a related embodiment, the above method includes a further step: (g) identifying a hydroxymethylation pattern in the 5hmC-containing DNA removed from the cell-free DNA in step (b). This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with OGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises stool, tissue sample (e.g., skin tissue), an organ secretion, CSF, saliva, blood, or urine. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events. The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

It is contemplated that embodiments of the technology are provided in the form of a kit. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

Various cancers are predicted by various combinations of markers, e.g., as identified by statistical techniques related to specificity and sensitivity of prediction. The technology provides methods for identifying predictive combinations and validated predictive combinations for some cancers.

### Methods

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker comprising a DMR (e.g., DMR 1-331 e.g., as provided in Tables 1A, 2A, 7A, 8A, and 9) and
2) detecting melanoma, metastatic melanoma, or primary cutaneous melanoma (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker selected from a chromosomal region having an annotation selected from the group consisting of c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33, and
2) detecting melanoma (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker selected from a chromosomal region having an annotation selected from the group consisting of MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763, and
2) detecting melanoma (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker selected from a chromosomal region having an annotation selected from the group consisting of AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73, and
2) detecting melanoma (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker selected from a chromosomal region having an annotation selected from the group consisting of MAX.chr20.21080958-21081038, MAX.chr7.155259597-155259763, FOXL2NB, and HOXA9_A, and
2) detecting metastatic melanoma (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma, fine needle aspirate, deep tissue, or skin tissue) obtained from the subject with at least one reagent or series of reagents that distinguishes between methylated and non-methylated CpG dinucleotides within at least one marker selected from a chromosomal region having an annotation selected from the group consisting of MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763, and
2) detecting metastatic melanoma (e.g., afforded with a sensitivity of greater than or equal to 80% and a specificity of greater than or equal to 80%).

In some embodiments of the technology, methods are provided that comprise the following steps:

1) measuring a methylation level for one or more genes in a biological sample of a human individual through treating genomic DNA in the biological sample with a reagent that modifies DNA in a methylation-specific manner (e.g., wherein the reagent is a bisulfite reagent, a methylation-sensitive restriction enzyme, or a methylation-dependent restriction enzyme), wherein the one or more genes is selected from one of the following groups:
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, 5B and 6; Example I);
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11);
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B; Example II);
One or more markers recited in Tables 8A and 8B (Example II); and
MAX.chr20.21080958-21081038, MAX.chr7.155259597-155259763, FOXL2NB, and HOXA9_A (see, Table 5C, Example I);

2) amplifying the treated genomic DNA using a set of primers for the selected one or more genes; and

3) determining the methylation level of the one or more genes by polymerase chain reaction, nucleic acid sequencing, mass spectrometry, methylation-specific nuclease, mass-based separation, and target capture.

In some embodiments of the technology, methods are provided that comprise the following steps:

1) measuring an amount of at least one methylated marker gene in DNA from the sample, wherein the one or more genes is selected from one of the following groups:
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, 5B and 6; Example I;
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11);
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B; Example II);
One or more markers recited in Tables 8A and 8B (Example II); and
MAX.chr20.21080958-21081038, MAX.chr7.155259597-155259763, FOXL2NB, and HOXA9_A (see, Table 5C, Example I);

2) measuring the amount of at least one reference marker in the DNA; and

3) calculating a value for the amount of the at least one methylated marker gene measured in the DNA as a percentage of the amount of the reference marker gene measured in the DNA, wherein the value indicates the amount of the at least one methylated marker DNA measured in the sample.

In some embodiments of the technology, methods are provided that comprise the following steps:

1) measuring a methylation level of a CpG site for one or more genes in a biological sample of a human individual through treating genomic DNA in the biological sample with bisulfite a reagent capable of modifying DNA in a methylation-specific manner (e.g., a methylation-sensitive restriction enzyme, a methylation-dependent restriction enzyme, and a bisulfite reagent);

2) amplifying the modified genomic DNA using a set of primers for the selected one or more genes; and

3) determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
wherein the one or more genes is selected from one of the following groups:
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 (see, Tables 4, 5A, 5B and 6; Example I;
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763 (see, Tables 9, 10 and 11);
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73 (see, Tables 7A and 7B; Example II);
One or more markers recited in Tables 8A and 8B (Example II); and
MAX.chr20.21080958-21081038, MAX.chr7.155259597-155259763, FOXL2NB, and HOXA9_A (see, Table 5C, Example I).

Within any of such methods, determining the methylation level for any of such markers is accomplished with the primers recited in Table 3.

Preferably, the sensitivity for such methods is from about 70% to about 100%, or from about 80% to about 90%, or from about 80% to about 85%. Preferably, the specificity is from about 70% to about 100%, or from about 80% to about 90%, or from about 80% to about 85%.

Genomic DNA may be isolated by any means, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, skin tissue, lymphatic tissue or aspirate, brain, lung, liver, bone or other deep organ tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a skin tissue sample or a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, that distinguishes between methylated and non-methylated CpG dinucleotides within at least one marker comprising a DMR (e.g., DMR 1-331, e.g., as provided by Tables 1A, 2A, 7A, 8A, and 9).

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from DMR 1-331, e.g., as provided in Tables 1A, 2A, 7A, 8A, and 9). The method of analysis may be selected from those known in the art, including those listed herein, e.g., QuARTS and MSP as described herein.

Aberrant methylation, more specifically hypermethylation of a marker comprising a DMR (e.g., DMR 1-331, e.g., as provided by Tables 1A, 2A, 7A, 8A, and 9) is associated with melanoma.

The technology relates to the analysis of any sample associated with melanoma. For example, in some embodiments the sample comprises a tissue (e.g., skin tissue) and/or biological fluid obtained from a patient. In some embodiments, the sample comprises a secretion. In some embodiments, the sample comprises blood, serum, plasma, gastric secretions, pancreatic juice, a gastrointestinal biopsy sample, microdissected cells from a skin tissue biopsy, deep tissue biopsy, fine needle aspirate, and/or cells recovered from stool. In some embodiments, the sample comprises skin tissue. In some embodiments, the subject is human. The sample may include cells, secretions, or tissues from the ovary, breast, liver, bile ducts, pancreas, stomach, colon, skin, rectum, esophagus, small intestine, appendix, duodenum, polyps, gall bladder, anus, lymph nodes, brain, bone, and/or peritoneum. In some embodiments, the sample comprises cellular fluid, ascites, urine, feces, pancreatic fluid, fluid obtained during endoscopy, blood, mucus, or saliva. In some embodiments, the sample is a stool sample. In some embodiments, skin cells are collected using adhesive tape or other adhesive surfaces.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens

In some embodiments, the technology relates to a method for treating a patient (e.g., a patient with melanoma) (e.g., a patient with one or more of metastatic melanoma, primary cutaneous melanoma), the method comprising determining the methylation state of one or more DMR as provided herein and administering a treatment to the patient based on the results of determining the methylation state. The treatment may be administration of a pharmaceutical compound, a vaccine, an immunotherapy, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments of the technology, a method for diagnosing melanoma in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker (e.g., a DMR as disclosed herein), the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of melanoma, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) in a biological sample over time during the course of an effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with melanoma risk, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR), the chance of a given outcome (e.g., suffering from an melanoma) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR) can be established, and the degree of change in the methylation state of the biamarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with metaplasia or from donors with a melanoma. In certain embodiments of the method, a subject is identified as having metaplasia upon identifying an aberrant methylation state of one or more DMR provided herein in a biological sample obtained from the subject. In other embodiments of the method, the detection of an aberrant methylation state of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having melanoma if, when compared to a control methylation state, there is a measurable difference in the methylation state of at least one biomarker in the sample. Conversely, when no change in methylation state is identified in the biological sample, the subject can be identified as not having melanoma, not being at risk for the cancer, or as having a low risk of the cancer. In this regard, subjects having the cancer or risk thereof can be differentiated from subjects having low to substantially no cancer or risk thereof. Those subjects having a risk of developing melanoma can be placed on a more intensive and/or regular screening schedule, including endoscopic surveillance. On the other hand, those subjects having low to substantially no risk may avoid being subjected to additional testing for melanoma (e.g., invasive procedure), until such time as a future screening, for example, a screening conducted in accordance with the present technology, indicates that a risk of melanoma has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, melanoma indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including race horses), and the like.

The presently-disclosed subject matter further includes a system for diagnosing melanoma and/or a specific form of melanoma (e.g., metastatic melanoma, primary cutaneous melanoma) in a subject. The system can be provided, for example, as a commercial kit that can be used to screen for a risk of melanoma or diagnose melanoma in a subject from whom a biological sample has been collected. An exemplary system provided in accordance with the present technology includes assessing the methylation state of a DMR as provided in Tables 1A, 2A, 7A, 8A, and 9.

### EXAMPLES

### Example I

### Materials and Methods

Tissue and blood were obtained from Mayo Clinic biospecimen repositories with institutional IRB oversight. Samples were chosen with strict adherence to subject research authorization and inclusion/exclusion criteria. Cancers consisted of 21 metastatic melanomas. Controls included 15 non-neoplastic skin epidermis samples, 16 benign melanocytic nevi, and 36 whole blood derived leukocytes. Tissues were macro-dissected and histology reviewed by an expert pathologist. Samples were age matched, randomized, and blinded. DNA was purified using the QIAamp DNA Tissue Mini kit and QIAamp DNA Blood Mini kit (Qiagen, Valencia Calif.), respectively. DNA was re-purified with AMPure XP beads (Beckman-Coulter, Brea Calif.) and quantified by PicoGreen (Thermo-Fisher, Waltham Mass.). DNA integrity was assessed using qPCR.

RRBS sequencing libraries were prepared following the Meissner protocol (see, Gu et al. Nature Protocols 2011 April; 6(4):468-81) with modifications. Samples were combined in a 4-plex format and sequenced by the Mayo Genomics Facility on the Illumina HiSeq 2500 instrument (Illumina, San Diego Calif.). Reads were processed by Illumina pipeline modules for image analysis and base calling. Secondary analysis was performed using SAAP-RRBS, a Mayo developed bioinformatics suite. Briefly, reads were cleaned-up using Trim-Galore and aligned to the GRCh37/hgl9 reference genome build with BSMAP. Methylation ratios were determined by calculating C/(C+T) or conversely, G/(G+A) for reads mapping to reverse strand, for CpGs with coverage ≥10× and base quality score ≥20.

Individual CpGs were ranked by hypermethylation ratio, namely the number of methylated cytosines at a given locus over the total cytosine count at that site. For cases, the ratios were required to be ≥0.20 (20%); for tissue controls, ≤0.05 (5%) tissue vs tissue analysis; ≥0.20 (20%) tissue vs buffy coat; for buffy coat controls, ≤0.01 (1%). CpGs which did not meet these criteria were discarded. Subsequently, candidate CpGs were binned by genomic location into DMRs (differentially methylated regions) ranging from approximately 40-220 bp with a minimum cut-off of 5 CpGs per region. DMRs with excessively high CpG density (>30%) were excluded to avoid GC-related amplification problems in the validation phase. For each candidate region, a 2-D matrix was created which compared individual CpGs in a sample-to-sample fashion for both cases and controls. These CpG matrices were then compared back to the reference sequence to assess whether genomically contiguous methylation sites had been discarded during the initial filtering. From this subset of regions, final selections required coordinated and contiguous hypermethylation (in cases) of individual CpGs across the DMR sequence on a per sample level. Conversely, control samples had to have at least 10-fold less methylation than cases and the CpG pattern had to be more random and less coordinated. At least 10% of cancer samples were required to have at least a 50% hypermethylation ratio for every CpG site within the DMR.

In a separate analysis, a proprietary DMR identification pipeline and regression package was utilized to derive DMRs based on average methylation values of the CpG. The difference in average methylation percentage was compared between malignant melanoma cases, tissue controls and buffy coat controls; a tiled reading frame within 100 base pairs of each mapped CpG was used to identify DMRs where control methylation was <5%; DMRs were only analyzed if the total depth of coverage was 10 reads per subject on average and the variance across subgroups was >0. Assuming a biologically relevant increase in the odds ratio of >3× and a coverage depth of 10 reads, ≥18 samples per group were required to achieve 80% power with a two-sided test at a significance level of 5% and assuming binomial variance inflation factor of 1.

Following regression, DMRs were ranked by p-value, area under the receiver operating characteristic curve (AUC) and fold-change difference between cases and all controls. No adjustments for false discovery were made during this phase as independent validation was planned a priori.

A subset of the DMRs was chosen for further development. The criteria were primarily the logistic-derived area under the ROC curve metric which provides a performance assessment of the discriminant potential of the region. An AUC of 0.85 was chosen as the cut-off. In addition, the methylation fold-change ratio (average cancer hypermethylation ratio/average control hypermethylation ratio) was calculated and a lower limit of 10 was employed for tissue vs tissue comparisons and 20 for the tissue vs buffy coat comparisons. P values were required to be less than 0.01. DMRs had to be listed in both the average and individual CpG selection processes. Quantitative methylation specific PCR (qMSP) primers were designed for candidate regions using MethPrimer (Li LC and Dahiya R. Bioinformatics 2002 November; 18(11):1427-31) and QC checked on 20 ng (6250 equivalents) of positive and negative genomic methylation controls. Multiple annealing temperatures were tested for optimal discrimination. Validation was performed in two stages of qMSP. The first consisted of re-testing the sequenced DNA samples. This was done to verify that the DMRs were truly discriminant and not the result of over-fitting the extremely large next generation sequencing (NGS) datasets. The second utilized a larger set of independent samples: metastatic melanoma from 35 patients, primary melanoma from 26 patients, and 73 control samples (47 benign precursor lesions or normal skin, and 26 healthy buffy coat samples.

Tissues were identified as before, with expert clinical and pathological review. DNA purification was performed using the Qiagen QIAmp FFPE tissue kit. The EZ-96 DNA Methylation kit (Zymo Research, Irvine Calif.) was used for the bisulfite conversion step. 10 ng of converted DNA (per marker) was amplified using SYBR Green detection on Roche 480 LightCyclers (Roche, Basel Switzerland). Serially diluted universal methylated genomic DNA (Zymo Research) was used as a quantitation standard. A CpG agnostic ACTB (0-actin) assay was used as an input reference and normalization control. Results were expressed as methylated copies (specific marker)/copies of ACTB.

Results were analyzed logistically for individual MDMs (methylated DNA marker) performance. For combinations of markers, two techniques were used. First, the rPart technique was applied to the entire MDM set and limited to combinations of 3 MDMs, upon which an rPart predicted probability of cancer was calculated. The second approach used random forest regression (rForest) which generated 500 individual rPart models that were fit to boot strap samples of the original data (roughly ⅔ of the data for training) and used to estimate the cross-validation error (⅓ of the data for testing) of the entire MDM panel and was repeated 500 times. to avoid spurious splits that either under- or overestimate the true cross-validation metrics. Results were then averaged across the 500 iterations.

### Results

A proprietary methodology of sample preparation, sequencing, analyses, and filters was utilized to identify and narrow differentially methylated regions (DMRs) to those which would pinpoint skin cancers and excel in a clinical testing environment. From the tissue-to-tissue analysis, 124 hypermethylated malignant melanomas (MM) DMRs were identified (Tables TA and 1B). They included MM specific regions as well as those regions that targeted a more universal cancer spectrum. The tissue to leukocyte (buffy coat) analysis yielded 38 hypermethylated MM+epidermis tissue DMRs with less than 1% noise in WBCs (Tables 2A and 2B).

From the tissue and buffy marker groups, 40 candidate markers were chosen for an initial pilot. Methylation-specific PCR assays were developed and tested on two rounds of tissue samples; those that were sequenced (frozen) and larger independent cohorts (FFPE). Short amplicon primers (<150 bp) were designed to target the most discriminant CpGs within a DMR and tested on controls to ensure that fully methylated fragments amplified robustly and in a linear fashion; that unmethylated and/or unconverted fragments did not amplify. The 80 primer sequences are listed in Table 3.

The results from stage one validation were analyzed logistically to determine AUC and fold change. The analyses for the tissue and buffy coat controls were run separately. Results are highlighted in Table 4. The degree of red shading indicates the discrimination strength of the marker assay. A number of assays were 100% discriminant in MM from buffy coat samples and one was 100% in the MM vs control tissue analysis.

These results provided a rich source of highly performing candidates to take into independent sample testing. Of the original 40 assays, 32 were selected. Most fell within the AUC range of 0.90-1.00, but others were included which had extremely high fold change (FC) numbers (very little background) and/or those which exhibited complementarity with other methylated DNA markers (MDMS). All assays demonstrated high analytical performance—linearity, efficiency, sequence specificity (assessed using melt curve analysis), and strong amplification.

In round 2 validation, as in the previous step, the entire sample and marker set was run in one batch. ˜10 ng of FFPE-derived sample DNA was run per marker—350 total. MM vs normal tissue and buffy coat results for individual MDMs are listed in Table 5A. On receiver operator characteristics analyses of individual marker candidates, areas under the curve (AUCs) for the MM vs control tissue comparison ranged from 0.43 to 0.97 (Table 5B); median AUC was 0.835. At 100% specificity, a cross-validated panel of 5 MDMs (MAX.chr20.21080958-21081038, MAX.chr7.155259597-155259763 (using primer SEQ ID Nos. 47 and 48), MAX.chr7.155259597-155259763 (using primer SEQ ID Nos. 49 and 50), FOXL2NB, and HOXA9_A) yielded a sensitivity of 33/35 cases (94.3% (95% CI, 86.6-100%)) for metastatic melanoma and 22/26 cases (84.6% (95% CI, 70.7-98.5%)) for primary melanoma (Table 5C). For the MM vs buffy coat comparison, AUCs ranged from 0.80 to 0.98 and methylation fold change ratios were >20 with a median of 64 (Table 6).

Whole methylome sequencing, stringent filtering criteria, and biological validation yielded outstanding candidate MDMs for malignant melanoma. A panel of five novel MDMs (MAX.chr20.21080958-21081038, MAX.chr7.155259597-155259763 (using primer SEQ ID Nos. 47 and 48), MAX.chr7.155259597-155259763 (using primer SEQ ID Nos. 49 and 50), FOXL2NB, and HOXA9_A) assayed on tissue and undetectable in normal buffy coat achieves very high discrimination between melanoma and benign control tissues.

TABLE 1A

| | | | DMR Start-End Positions |
|---|---|---|---|
| DMR No. | Gene Annotation | Chromosome No. | (GRCh37/hg19) |
| 1 | ACTR3C | 7 | 150020206-150020574 |
| 2 | ADAM8 | 10 | 135090076-135090566 |
| 3 | ALOX12B | 17 | 7976119-7976483 |
| 4 | ALX3 | 1 | 110612988-110613158 |
| 5 | ANKRD33B | 5 | 10565042-10565297 |
| 6 | ASB2 | 14 | 94405842-94406155 |
| 7 | ATP8B1 | 18 | 55469253-55469316 |
| 8 | BANK1 | 4 | 102711871-102711992 |
| 9 | BARHL1 | 9 | 135462616-135462735 |
| 10 | BOLL | 2 | 198651427-198651480 |
| 11 | BTBD19 | 1 | 45279368-45279564 |
| 12 | C10orf55 | 10 | 75670653-75670789 |
| 13 | C14orf50 | 14 | 65016722-65016815 |
| 14 | C1QL3 | 10 | 16562465-16562672 |
| 15 | C2orf82 | 2 | 233740970-233741417 |
| 16 | FOXL2NB | 3 | 138663981-138664076 |
| 17 | C6orf132 | 6 | 42109906-42110586 |
| 18 | CCDC109B | 4 | 110480758-110481153 |
| 19 | CD8B | 2 | 87089115-87089153 |
| 20 | CLDN10 | 13 | 96204805-96204874 |
| 21 | CLIC5 | 6 | 45982945-45983289 |
| 22 | CRHBP_A | 5 | 76249301-76249698 |
| 23 | CRHBP_B | 5 | 76249969-76250202 |
| 24 | CYP26A1 | 10 | 94834101-94834349 |
| 25 | DAB1 | 1 | 58715853-58716179 |
| 26 | DMRT3 | 9 | 977078-977505 |
| 27 | F2RL1 | 5 | 76115146-76115392 |
| 28 | FAM174B_A | 15 | 93198406-93198607 |
| 29 | FAM174B_B | 15 | 93199001-93199143 |
| 30 | FGFR1 | 8 | 38323133-38323199 |
| 31 | FLJ41350 | 10 | 102986651-102986744 |
| 32 | FOXP1 | 3 | 71630795-71630964 |
| 33 | GATA6 | 18 | 19745472-19745506 |
| 34 | GFRA2 | 8 | 21647026-21647253 |
| 35 | GIPC2 | 1 | 78511659-78511979 |
| 36 | GJA1 | 6 | 121758272-121758386 |
| 37 | GNA14 | 9 | 80263496-80263854 |
| 38 | GREM1 | 15 | 33010893-33011064 |
| 39 | GSN | 9 | 124062312-124062460 |
| 40 | GSTO2 | 10 | 106034646-106034802 |
| 41 | HEYL | 1 | 40105255-40105487 |
| 42 | HIST1H3G | 6 | 26273747-26273884 |
| 43 | HLA-J | 6 | 29974345-29974746 |
| 44 | HLF | 17 | 53343188-53343401 |
| 45 | HOPX | 4 | 57522436-57522653 |
| 46 | HOXA9_A | 7 | 27209628-27209739 |
| 47 | HOXB5_A | 17 | 46674939-46675240 |
| 48 | IGFBP5 | 2 | 217559020-217559578 |
| 49 | KCNQ4_A | 1 | 41284410-41284590 |
| 50 | LAMA3 | 18 | 21269793-21270254 |
| 51 | LBX2_A | 2 | 74725933-74726331 |
| 52 | LBX2_B | 2 | 74726449-74726634 |
| 53 | LOC100128977 | 17 | 43974551-43974611 |
| 54 | LOC648809_A | 15 | 84749077-84749238 |
| 55 | MAL_A | 2 | 95692316-95692525 |
| 56 | MAL_B | 2 | 95691099-95691322 |
| 57 | MAX.chr1.208132378-208132686 | 1 | 208132378-208132686 |
| 58 | MAX.chr1.212838668-212838781 | 1 | 212838668-212838781 |
| 59 | MAX.chr1.29101688-29101792 | 1 | 29101688-29101792 |
| 60 | MAX.chr1.32238359-32238526 | 1 | 32238359-32238526 |
| 61 | MAX.chr10.22541869-22541953 | 10 | 22541869-22541953 |
| 62 | MAX.chr10.22624234-22624571 | 10 | 22624234-22624571 |
| 63 | MAX.chr10.29011068-29011271 | 10 | 29011068-29011271 |
| 64 | MAX.chr10.62492690-62492812 | 10 | 62492690-62492812 |
| 65 | MAX.chr11.14926602-14927148 | 11 | 14926602-14927148 |
| 66 | MAX.chr13.29106812-29106917 | 13 | 29106812-29106917 |
| 67 | MAX.chr13.33924444-33924575 | 13 | 33924444-33924575 |
| 68 | MAX.chr15.53097592-53097737 | 15 | 53097592-53097737 |
| 69 | MAX.chr15.53098014-53098109 | 15 | 53098014-53098109 |
| 70 | MAX.chr16.21675437-21675543 | 16 | 21675437-21675543 |
| 71 | MAX.chr17.29335857-29336076 | 17 | 29335857-29336076 |
| 72 | MAX.chr17.6559968-6560221 | 17 | 6559968-6560221 |
| 73 | MAX.chr17.73073682-73073830 | 17 | 73073682-73073830 |
| 74 | MAX.chr2.162283698-162283742 | 2 | 162283698-162283742 |
| 75 | MAX.chr2.223170699-223170942 | 2 | 223170699-223170942 |
| 76 | MAX.chr2.233285553-233285863 | 2 | 233285553-233285863 |
| 77 | MAX.chr2.237082545-237082555 | 2 | 237082545-237082555 |
| 78 | MAX.chr2.97193120-97193287 | 2 | 97193120-97193287 |
| 79 | MAX.chr2.97193478-97193562 | 2 | 97193478-97193562 |
| 80 | MAX.chr20.21491437-21491503 | 20 | 21491437-21491503 |
| 81 | MAX.chr20.3229151-3229791 | 20 | 3229151-3229791 |
| 82 | MAX.chr4.4867535-4867655 | 4 | 4867535-4867655 |
| 83 | MAX.chr5.139144115-139144199 | 5 | 139144115-139144199 |
| 84 | MAX.chr5.42952363-42952548 | 5 | 42952363-42952548 |
| 85 | MAX.chr5.42992655-42992768 | 5 | 42992655-42992768 |
| 86 | MAX.chr5.60921627-60921853 | 5 | 60921627-60921853 |
| 87 | MAX.chr5.76476081-76476350 | 5 | 76476081-76476350 |
| 88 | MAX.chr5.77268600-77268725 | 5 | 77268600-77268725 |
| 89 | MAX.chr6.157557371-157557657 | 6 | 157557371-157557657 |
| 90 | MAX.chr6.26234019-26234258 | 6 | 26234019-26234258 |
| 91 | MAX.chr6.29521537-29521696 | 6 | 29521537-29521696 |
| 92 | MAX.chr6.45631468-45631500 | 6 | 45631468-45631500 |
| 93 | MAX.chr6.99295949-99295996 | 6 | 99295949-99295996 |
| 94 | MAX.chr7.149120005-149120361 | 7 | 149120005-149120361 |
| 95 | MAX.chr7.156409579-156409711 | 7 | 156409579-156409711 |
| 96 | MAX.chr8.82543201-82543257 | 8 | 82543201-82543257 |
| 97 | ME3 | 11 | 86382754-86383237 |
| 98 | MGA | 15 | 41952429-41953195 |
| 99 | MIR155HG | 21 | 26934273-26934633 |
| 100 | MYO5A | 15 | 52821789-52821964 |
| 101 | NPR3 | 5 | 32713578-32713695 |
| 102 | NRARP | 9 | 140200138-140200258 |
| 103 | OLIG2 | 21 | 34395395-34395485 |
| 104 | OSR2 | 8 | 99952116-99952873 |
| 105 | OXT | 20 | 3052495-3052618 |
| 106 | OXTR | 3 | 8809811-8809899 |
| 107 | PLEKHA7 | 11 | 17035319-17035436 |
| 108 | PROM1_A | 4 | 16084793-16085386 |
| 109 | RGMA | 15 | 93632651-93632755 |
| 110 | RNF220_A | 1 | 44883618-44883741 |
| 111 | SIX4_A | 14 | 61188480-61188614 |
| 112 | SIX4_B | 14 | 61188901-61189191 |
| 113 | SLC5A2 | 16 | 31498792-31499039 |
| 114 | STAT4 | 2 | 192015272-192015447 |
| 115 | SULT1A1 | 16 | 28634532-28634986 |
| 116 | SYNPO | 5 | 150004289-150004715 |
| 117 | TAL1 | 1 | 47698036-47698142 |
| 118 | TLX1NB | 10 | 102881103-102881247 |
| 119 | TMEM30B | 14 | 61747319-61748246 |
| 120 | TNFRSF10C | 8 | 22960641-22960743 |
| 121 | TNRC18 | 7 | 5467547-5467777 |
| 122 | TSPAN33 | 7 | 128809044-128809129 |
| 123 | VIPR2 | 7 | 158938034-158938137 |
| 124 | ZIC1 | 3 | 147130252-147130259 |

TABLE 1B

| | | Area Under | | |
|---|---|---|---|---|
| DMR No. | Gene Annotation | Curve | Fold-Change | p-value |
| 1 | ACTR3C | 0.89 | 23.83 | 0.001078 |
| 2 | ADAM8 | 0.8867 | 51.35 | 0.006575 |
| 3 | ALOX12B | 0.95 | 21.28 | 8.69E−05 |
| 4 | ALX3 | 0.9368 | 20.17 | 2.50E−06 |
| 5 | ANKRD33B | 0.8558 | 56.29 | 0.002555 |
| 6 | ASB2 | 0.8667 | 18.82 | 0.000224 |
| 7 | ATP8B1 | 0.956 | 22.36 | 0.009388 |
| 8 | BANK1 | 0.8474 | 14.09 | 7.01E−05 |
| 9 | BARHL1 | 0.8944 | 21.63 | 5.90E−06 |
| 10 | BOLL | 0.8995 | 4.667 | 0.002879 |
| 11 | BTBD19 | 0.9283 | 16.44 | 1.08E−05 |
| 12 | C10orf55 | 0.9561 | 68.02 | 0.003481 |
| 13 | C14orf50 | 0.875 | 145.4 | 0.003345 |
| 14 | C1QL3 | 0.8481 | 19.95 | 1.01E−05 |
| 15 | C2orf82 | 0.9333 | 157.4 | 0.001918 |
| 16 | FOXL2NB | 0.98 | 17.62 | 2E−06 |
| 17 | C6orf132 | 0.9016 | 37.73 | 0.004609 |
| 18 | CCDC109B | 0.8467 | 35.52 | 0.003198 |
| 19 | CD8B | 0.8462 | 31.39 | 0.002657 |
| 20 | CLDN10 | 0.9364 | 16.41 | 0.004922 |
| 21 | CLIC5 | 0.9 | 34.97 | 0.008759 |
| 22 | CRHBP_A | 0.8833 | 9.946 | 0.000229 |
| 23 | CRHBP_B | 0.9033 | 10.97 | 0.00095 |
| 24 | CYP26A1 | 0.9298 | 18.73 | 0.000592 |
| 25 | DAB1 | 0.9433 | 33.88 | 9.19E−06 |
| 26 | DMRT3 | 0.8632 | 14.94 | 0.000788 |
| 27 | F2RL1 | 0.9397 | 36.65 | 0.002797 |
| 28 | FAM174B_A | 0.9762 | 62.26 | 1.70E−05 |
| 29 | FAM174B_B | 0.9463 | 256.7 | 0.000721 |
| 30 | FGFR1 | 0.8778 | 15.68 | 0.000343 |
| 31 | FLJ41350 | 0.913 | 43.78 | 0.002036 |
| 32 | FOXP1 | 0.9333 | 101.5 | 0.007177 |
| 33 | GATA6 | 0.9515 | 22.69 | 0.000455 |
| 34 | GFRA2 | 0.9263 | 37.03 | 0.004077 |
| 35 | GIPC2 | 0.9183 | 12.7 | 3.40E−05 |
| 36 | GJA1 | 0.86 | 58.5 | 0.00201 |
| 37 | GNA14 | 0.9296 | 33.88 | 0.000511 |
| 38 | GREM1 | 0.9373 | 30.76 | 0.000847 |
| 39 | GSN | 0.9587 | 100.8 | 8.70E−05 |
| 40 | GSTO2 | 0.94 | 11.91 | 0.000835 |
| 41 | HEYL | 0.8737 | 27.66 | 0.000189 |
| 42 | HIST1H3G | 0.9526 | 58.48 | 0.003287 |
| 43 | HLA-J | 0.8783 | 47.28 | 0.004566 |
| 44 | HLF | 0.8533 | 47.04 | 0.002383 |
| 45 | HOPX | 0.907 | 32.71 | 0.002557 |
| 46 | HOXA9_A | 0.9667 | 26.5 | 0.000298 |
| 47 | HOXB5_A | 0.8667 | 39.78 | 0.008676 |
| 48 | IGFBP5 | 0.8667 | 32.37 | 0.002762 |
| 49 | KCNQ4_A | 0.9361 | 19.46 | 9.99E−05 |
| 50 | LAMA3 | 0.9167 | 20.02 | 0.000165 |
| 51 | LBX2_A | 0.89 | 49.07 | 0.003008 |
| 52 | LBX2_B | 0.8767 | 29.9 | 0.000206 |
| 53 | LOC100128977 | 0.8533 | 20.47 | 7.32E−05 |
| 54 | LOC648809_A | 0.8556 | 22.83 | 0.008257 |
| 55 | MAL_A | 0.9228 | 19.76 | 0.005503 |
| 56 | MAL_B | 0.869 | 15.87 | 0.005458 |
| 57 | MAX.chr1.208132378-208132686 | 0.9365 | 38.3 | 4.73E−06 |
| 58 | MAX.chr1.212838668-212838781 | 0.8609 | 17.15 | 0.0008 |
| 59 | MAX.chr1.29101688-29101792 | 0.891 | 76.06 | 0.004054 |
| 60 | MAX.chr1.32238359-32238526 | 0.9228 | 25.66 | 9.17E−05 |
| 61 | MAX.chr10.22541869-22541953 | 0.9246 | 20.45 | 1.47E−05 |
| 62 | MAX.chr10.22624234-22624571 | 0.9317 | 54.86 | 0.000785 |
| 63 | MAX.chr10.29011068-29011271 | 0.9082 | 52.59 | 0.003768 |
| 64 | MAX.chr10.62492690-62492812 | 1 | 77.97 | 0.000239 |
| 65 | MAX.chr11.14926602-14927148 | 0.9967 | 40.58 | 1.49E−07 |
| 66 | MAX.chr13.29106812-29106917 | 0.9 | 34.6 | 0.001974 |
| 67 | MAX.chr13.33924444-33924575 | 0.9533 | 89.4 | 0.00172 |
| 68 | MAX.chr15.53097592-53097737 | 0.8632 | 48.66 | 5.53E−06 |
| 69 | MAX.chr15.53098014-53098109 | 0.9544 | 66.75 | 3.08E−06 |
| 70 | MAX.chr16.21675437-21675543 | 0.8988 | 60.5 | 0.001252 |
| 71 | MAX.chr17.29335857-29336076 | 0.9333 | 24.19 | 0.000405 |
| 72 | MAX.chr17.6559968-6560221 | 0.9396 | 62.27 | 0.004068 |
| 73 | MAX.chr17.73073682-73073830 | 0.955 | 36.76 | 2.19E−06 |
| 74 | MAX.chr2.162283698-162283742 | 0.9363 | 19.6 | 0.000364 |
| 75 | MAX.chr2.223170699-223170942 | 0.8967 | 86.51 | 0.00616 |
| 76 | MAX.chr2.233285553-233285863 | 0.8902 | 21.11 | 0.000109 |
| 77 | MAX.chr2.237082545-237082555 | 0.86 | 12.65 | 0.00343 |
| 78 | MAX.chr2.97193120-97193287 | 0.8933 | 39.54 | 0.000521 |
| 79 | MAX.chr2.97193478-97193562 | 0.8575 | 18.58 | 0.000535 |
| 80 | MAX.chr20.21491437-21491503 | 0.8846 | 61.09 | 0.000148 |
| 81 | MAX.chr20.3229151-3229791 | 0.9067 | 24.62 | 0.000979 |
| 82 | MAX.chr4.4867535-4867655 | 0.8929 | 7.205 | 0.00046 |
| 83 | MAX.chr5.139144115-139144199 | 0.8526 | 20.95 | 6.21E−05 |
| 84 | MAX.chr5.42952363-42952548 | 0.8467 | 10.55 | 0.00043 |
| 85 | MAX.chr5.42992655-42992768 | 0.9118 | 17.28 | 0.00023 |
| 86 | MAX.chr5.60921627-60921853 | 0.9433 | 18.5 | 2.20E−05 |
| 87 | MAX.chr5.76476081-76476350 | 0.8933 | 29.09 | 0.008773 |
| 88 | MAX.chr5.77268600-77268725 | 0.8526 | 28.45 | 8.93E−05 |
| 89 | MAX.chr6.157557371-157557657 | 0.87 | 23.15 | 0.000273 |
| 90 | MAX.chr6.26234019-26234258 | 0.8567 | 36.88 | 0.003721 |
| 91 | MAX.chr6.29521537-29521696 | 0.9684 | 45.37 | 7.01E−06 |
| 92 | MAX.chr6.45631468-45631500 | 0.8833 | 15.97 | 8.12E−06 |
| 93 | MAX.chr6.99295949-99295996 | 0.945 | 12.04 | 4.07E−08 |
| 94 | MAX.chr7.149120005-149120361 | 0.9333 | 21.53 | 1.37E−05 |
| 95 | MAX.chr7.156409579-156409711 | 0.8907 | 11.48 | 0.000132 |
| 96 | MAX.chr8.82543201-82543257 | 0.8947 | 24.02 | 1.11E−06 |
| 97 | ME3 | 0.9083 | 73.59 | 0.000717 |
| 98 | MGA | 0.87 | 19.37 | 1.86E−05 |
| 99 | MIR155HG | 0.9222 | 112.8 | 0.001734 |
| 100 | MYO5A | 0.8867 | 12.53 | 0.000599 |
| 101 | NPR3 | 0.9263 | 64.33 | 0.008733 |
| 102 | NRARP | 0.8947 | 42.84 | 0.000174 |
| 103 | OLIG2 | 0.9323 | 56.78 | 0.003995 |
| 104 | OSR2 | 0.8933 | 37.47 | 0.00343 |
| 105 | OXT | 0.9333 | 33.68 | 0.008098 |
| 106 | OXTR | 0.9431 | 123.4 | 0.00227 |
| 107 | PLEKHA7 | 0.8929 | 44.02 | 0.00434 |
| 108 | PROM1_A | 1 | 42.96 | 1.89E−06 |
| 109 | RGMA | 0.8833 | 23.15 | 0.000483 |
| 110 | RNF220_A | 0.8491 | 14.86 | 0.000128 |
| 111 | SIX4_A | 0.9433 | 111.7 | 0.000194 |
| 112 | SIX4_B | 0.8667 | 37.88 | 0.001949 |
| 113 | SLC5A2 | 0.99 | 19.33 | 6.60E−06 |
| 114 | STAT4 | 0.855 | 66.87 | 0.002125 |
| 115 | SULT1A1 | 0.8883 | 55.01 | 0.003744 |
| 116 | SYNPO | 0.89 | 81.82 | 0.003408 |
| 117 | TAL1 | 0.93 | 23.27 | 0.000142 |
| 118 | TLX1NB | 0.9317 | 51.91 | 0.000246 |
| 119 | TMEM30B | 1 | 33.4 | 0.000109 |
| 120 | TNFRSF10C | 0.8532 | 64.33 | 0.002406 |
| 121 | TNRC18 | 0.8632 | 90.35 | 0.003961 |
| 122 | TSPAN33 | 0.9489 | 159.8 | 0.008218 |
| 123 | VIPR2 | 0.9404 | 16.65 | 0.00045 |
| 124 | ZIC1 | 0.9333 | 24.87 | 5.79E−05 |

TABLE 2A

| | | | DMR Start-End Positions |
|---|---|---|---|
| DMR No. | Gene Annotation | Chromosome No. | (GRCh37/hg19) |
| 125 | ACSL5 | 10 | 114136053-114136132 |
| 126 | AMN | 14 | 103394740-103395037 |
| 127 | C17orf46_A | 17 | 43339287-43339498 |
| 128 | C17orf57_A | 17 | 45500997-45501102 |
| 129 | C1orf177 | 1 | 55266707-55266944 |
| 130 | C2CD4D_A | 1 | 151810637-151810661 |
| 131 | CBX4 | 17 | 77815562-77815704 |
| 132 | CD14 | 5 | 140012023-140012386 |
| 133 | COL2A1 | 12 | 48398305-48398375 |
| 134 | ECEL1 | 2 | 233352602-233352702 |
| 135 | FABP5 | 8 | 82192408-82192921 |
| 136 | FLJ22536_A | 6 | 21666448-21666575 |
| 137 | FRMD4A | 10 | 13933763-13933786 |
| 138 | GALNT3_A | 2 | 166650352-166650518 |
| 139 | GPR135_A | 14 | 59931111-59931142 |
| 140 | HOXB5_B | 17 | 46670918-46670995 |
| 141 | ITPKA | 15 | 41787475-41787780 |
| 142 | JSRP1 | 19 | 2253201-2253376 |
| 143 | KREMEN1_A | 22 | 29467629-29467723 |
| 144 | LMX1B | 9 | 129377720-129377822 |
| 145 | LOC648809_B | 15 | 84748833-84748932 |
| 146 | MAX.chr1.110627096-110627364 | 1 | 110627096-110627364 |
| 147 | MAX.chr1.162792353-162792404 | 1 | 162792353-162792404 |
| 148 | MAX.chr12.30975740-30975961 | 12 | 30975740-30975961 |
| 149 | MAX.chr13.20702971-20703054 | 13 | 20702971-20703054 |
| 150 | MAX.chr2.233741343-233741417 | 2 | 233741343-233741417 |
| 151 | MAX.chr2.71116033-71116122 | 2 | 71116033-71116122 |
| 152 | MAX.chr20.21080958-21081038 | 20 | 21080958-21081038 |
| 153 | MAX.chr7.155259597-155259763 | 7 | 1552596597-155259763 |
| 154 | MIR375 | 2 | 219866541-219866574 |
| 155 | MPZ_A | 1 | 161275561-161275653 |
| 156 | NFATC2_A | 20 | 50159073-50159168 |
| 157 | OCA2 | 15 | 28339956-28340185 |
| 158 | RNF207_A | 1 | 6266128-6266210 |
| 159 | RUNX2 | 6 | 45387427-45387521 |
| 160 | SCARF2 | 22 | 20785289-20785580 |
| 161 | SLC17A9 | 20 | 61585727-61585991 |
| 162 | STX16 | 20 | 57224798-57225227 |

TABLE 2B

| | | Area Under | | |
|---|---|---|---|---|
| DMR No. | Gene Annotation | Curve | Fold-Change | p-value |
| 125 | ACSL5 | 0.9637 | 119.6 | 0.000939 |
| 126 | AMN | 1 | 287.7 | 0.000602 |
| 127 | C17orf46_A | 1 | 220.5 | 7.92E−05 |
| 128 | C17orf57_A | 1 | 254.7 | 0.000177 |
| 129 | C1orf177 | 0.9552 | 86.88 | 0.000371 |
| 130 | C2CD4D_A | 1 | 182.9 | 3.88E−05 |
| 131 | CBX4 | 0.9825 | 169.4 | 7.25E−05 |
| 132 | CD14 | 0.9971 | 179 | 3.13E−05 |
| 133 | COL2A1 | 0.9624 | 349.3 | 0.000207 |
| 134 | ECEL1 | 0.954 | 94.35 | 0.005869 |
| 135 | FABP5 | 0.9778 | 93.12 | 0.001475 |
| 136 | FLJ22536_A | 0.9597 | 277.3 | 2.48E−05 |
| 137 | FRMD4A | 0.963 | 237 | 0.000311 |
| 138 | GALNT3_A | 1 | 412.1 | 0.000257 |
| 139 | GPR135_A | 1 | 121.4 | 0.000321 |
| 140 | HOXB5_B | 1 | 242.7 | 8.48E−06 |
| 141 | ITPKA | 0.9965 | 122.6 | 3.92E−06 |
| 142 | JSRP1 | 0.9861 | 147.3 | 0.000618 |
| 143 | KREMEN1_A | 0.9641 | 277.1 | 2.47E−05 |
| 144 | LMX1B | 1 | 153.7 | 7.63E−05 |
| 145 | LOC648809_B | 1 | 138.3 | 0.008842 |
| 146 | MAX.chr1.110627096-110627364 | 0.9912 | 268.8 | 7.46E−05 |
| 147 | MAX.chr1.162792353-162792404 | 0.9667 | 105.2 | 0.000223 |
| 148 | MAX.chr12.30975740-30975961 | 0.9569 | 191.3 | 1.57E−05 |
| 149 | MAX.chr13.20702971-20703054 | 0.9542 | 139.3 | 0.002903 |
| 150 | MAX.chr2.233741343-233741417 | 0.969 | 130.8 | 0.000742 |
| 151 | MAX.chr2.71116033-71116122 | 1 | 142.4 | 6.71E−06 |
| 152 | MAX.chr20.21080958-21081038 | 1 | 505.8 | 0.00042 |
| 153 | MAX.chr7.155259597-155259763 | 1 | 217.8 | 5.74E−05 |
| 154 | MIR375 | 0.9967 | 204.7 | 2.68E−06 |
| 155 | MPZ_A | 1 | 475.6 | 0.001579 |
| 156 | NFATC2_A | 0.9938 | 275.9 | 0.002118 |
| 157 | OCA2 | 0.9894 | 82.08 | 1.18E−06 |
| 158 | RNF207_A | 0.9958 | 197.1 | 0.000713 |
| 159 | RUNX2 | 0.9917 | 197.7 | 5.71E−05 |
| 160 | SCARF2 | 0.9795 | 342.6 | 0.009978 |
| 161 | SLC17A9 | 0.9622 | 118.1 | 0.00507 |
| 162 | STX16 | 1 | 1087 | 0.000123 |

TABLE 3

| | | Seq | | Seq | |
|---|---|---|---|---|---|
| DMR | | ID | Forward Primer 5′-3′ | ID | Reverse Primer 5′-3′ |
| No. | Gene Annotation | No. | Sequence | No. | Sequence |
| 12 | C10orf55 | 1 | TTAGGTGTATGGGAGG | 2 | AAAAAAAACGCGATCT |
| | | | AAGTACGGA | | CCAACGAAA |
| 15 | C2orf82 | 3 | CGTCGHUGHGGTT | 4 | ACCGAACGCGACCCC |
| | | | ATTTGCGTG | | TTTATTCG |
| 16 | FOXL2NB | 5 | TATGGATTGTACGGTA | 6 | ACGCAAACCTCTTAAC |
| | | | GTCGGGCGG | | CTTCTCTCACAAATCG |
| 21 | CLIC5 | 7 | GGCGTTGGTTTGCGG | 8 | CACCCACCAAAACTCG |
| | | | AAAGGTTAC | | AAATACGCT |
| 29 | FAM174B_B | 9 | TCGTCGTCGTTATTAT | 10 | ATAACCTACCCCGCCC |
| | | | TAATACGTT | | ACGT |
| 136 | FLJ22536_A | 11 | GAGTTTGGTAGTGGGA | 12 | CCGAAACGAACTAAAA |
| | | | GGAGATCGT | | CTACTCGAT |
| 32 | FOXP1 | 13 | GGGGCGATTTTTAGTA | 14 | CTACCCTCTCTATCCT |
| | | | AGTTTTTTTCGT | | AACCCCTCGAC |
| 138 | GALNT3_A | 15 | TCGGGATTTTTTTAGG | 16 | GAAAAAACGACCGCA |
| | | | GGTAATCGA | | AAAATTCACGTA |
| 45 | HOPX | 17 | TTGTATTTTTGTTTGCG | 18 | CCGAAAATCGATAAAA |
| | | | ACGGGGGC | | ACCCGCGAA |
| 46 | HOXA9_A | 19 | TTTTTTTTAGTTGTTCG | 20 | GACACTCCTTAAAACC |
| | | | TTCGTCGG | | ACGCGTT |
| 141 | ITPKA | 21 | GGGTTTATAAGTTCGG | 22 | CACCCAACACCTAACG |
| | | | AGGTCGA | | ACGA |
| 49 | KCNQ4_A | 23 | GGGCGAGGTTAGAGG | 24 | GCCTCCTACTAAAACT |
| | | | GGTTGTTHC | | CCAACCCCCG |
| 144 | LMX1B | 25 | ACGTCGCGTATTGTAA | 26 | CTTCCTAATACGAATC |
| | | | ATATTTTTCGTG | | AACGAATCGTCC |
| 146 | MAX.chr1.1106270 | 27 | CGAAAGATCGATCGAT | 28 | TTATAACGACGAATTC |
| | 96-110627364 | | TGTTCGACGG | | CGAAA |
| 61 | MAX.chr10.225418 | 29 | CGGTCGGAATTTCGGT | 30 | AACACTAACCGCGCCT |
| | 69-22541953 | | TTTCGC | | AATATCGCTA |
| 64 | MAX.chr10.624926 | 31 | CGGGATAGAGGCGAG | 32 | GAATATTTCCAAACAA |
| | 90-62492812 | | AGTTCGAATTC | | AAACCCCAAATCCTCG |
| 66 | MAX.chr13.291068 | 33 | CGGTTTTGATTTTAGG | 34 | TTAACTATCGAAAACG |
| | 12-29106917 | | GCGT | | ATTTCCGCGAA |
| 73 | MAX.chr17.730736 | 35 | TTTTTCGAGTCGTTTTA | 36 | GAACTCCGAACGCCG |
| | 82-73073830 | | TTTCGCGG | | CTTAAACGTA |
| 151 | MAX.chr2.7111603 | 37 | ATTTTTGATTATGGGTT | 38 | GAATTCACCTCGCCCC |
| | 3-71116122 | | TTGGTCGG | | TCGCT |
| 152 | MAX.chr20.210809 | 39 | TAAATAAAATATTGGTT | 40 | CGAACTAACGCTAAAC |
| | 58-21081038 | | TGTAGACGGACGCGG | | TCGCGCGAT |
| Me | MAX.chr5.4299265 | 41 | GGGGCGATAGTATTTA | 42 | TCACCCCAACTAAAAA |
| | 5-42992768 | | GGTCGTCGA | | AACTCCGAA |
| 86 | MAX.chr5.6092162 | 43 | TCGTAGAGGTTATCGG | 44 | AATACCCGAACCACAA |
| | 7-60921853 | | ATATAGCGA | | AACCCGCC |
| 91 | MAX.chr6.2952153 | 45 | TTGCGGAGGCGACGG | 46 | CAAAATAACCACGCGA |
| | 7-29521696 | | AGATATTATC | | ACGACGAA |
| 153 | MAX.chr7.1552595 | 47 | TCGTAGAGGGCGGAT | 48 | CGACCGAAAAACAAC |
| | 97-155259763 (v1) | | ATTAAATTAGCGG | | GTTTATTCGCT |
| 153 | MAX.chr7.1552595 | 49 | TTTTCGGCGGAGTCG | 50 | GATCTCCGCTCGCCT |
| | 97-155259763 (v2) | | GGATTTTTTC | | CGACG |
| 97 | ME3 | 51 | GTTGCGGGGTATCGG | 52 | CCTACCCTCTCCCAAA |
| | | | GTTTGATTC | | AAACGCGTC |
| 99 | MIR155HG (v1) | 53 | CGGATAGCGGAGTTTC | 54 | AAACGTCTCCTTAATT |
| | | | GAGTCGTTC | | CCCCGCGCTT |
| 99 | MIR155HG (v2) | 55 | TAAGCGCGGGGAATTA | 56 | CGAAAACGCGAAACTA |
| | | | AGGAGACGT | | AAATCGACGTA |
| 156 | NFATC2_A | 57 | GTTGGCGGAGGCGGT | 58 | CGACGTACGTCCCTA |
| | | | TCGAG | | CGAAA |
| 157 | OCA2 | 59 | ATTTGTTTTGTCGGGA | 60 | CAAATAACCCAAACTC |
| | | | AGGGGACGG | | CTATACGCA |
| 105 | OXT | 61 | CGTTTGAGAATTTTAG | 62 | CGAAACAACGAAACTA |
| | | | GAGTTGAGCGG | | AAACGCGCT |
| 158 | RNF207_A | 63 | GAGGAGAGGTAGGAG | 64 | AATAATTCCCACTCTA |
| | | | AGGTTACGG | | CGCGAA |
| 159 | RUNX2 | 65 | TCGAAAAGATAATTAA | 66 | CGAAAACCCATTTAAC |
| | | | AAATCGTACGCGT | | CAAACCGAA |
| 111 | SIX4_A | 67 | GGTGGTTCGGCGTAAT | 68 | CGTACGCCTTCCGCA |
| | | | TAAAGCGT | | AATATACGAA |
| 162 | STX16 | 69 | TTTTACGTAGAAATAAA | 70 | AAAAAACCGAAACCCC |
| | | | GGACGCGT | | AAAAACGAC |
| 117 | TAL1 (v1) | 71 | GTTCGTTTTCGATAAG | 72 | AATCCCCACTCCCTCC |
| | | | CGTTTCGGT | | GATA |
| 117 | TAL1 (v2) | 73 | AAGGTATTGTCGCGG | 74 | TAAAATAAATCATTTAA |
| | | | GTTCGTTCGT | | CCCATAATAACCGAA |
| 119 | TMEM30B (v1) | 75 | GGTTTAGGTCGATGAA | 76 | CTATCGACCAACATCG |
| | | | GGTTAGGTTCGCGTA | | CGCTACCG |
| 119 | TMEM30B (v2) | 77 | TTAGTTTCGTTTTCGTT | 78 | AAATACGACTCCCGAT |
| | | | TAGGTGCGTTG | | AACCCGCGA |
| 122 | TSPAN33 | 79 | CGGTTGTAGGGAGGA | 80 | ACGCCAAAAAACCCAA |
| | | | TTTCGAGGAAGTTC | | CGCA |

TABLE 4

| | | AUC Malignant | | | |
|---|---|---|---|---|---|
| | | Melanoma Tissue | | | |
| | | (MM) vs Benign | | | |
| | | Melanocytic | | | |
| | | Nevi Tissue (Nevi) | | | |
| | | and Normal | AUC MM | AUC MM | AUC Nevi |
| DMR No. | Gene Annotation | Tissue (Normal) | vs Buffy Coat | vs Nevi | vs Normal |
| 64 | MAX.chr10.62492690- | 0.81773 | 0.95971 | 0.81633 | 0.42381 |
| | 62492812 | | | | |
| 119 | TMEM30B (with primer | 0.80624 | 0.91941 | 0.86224 | 0.68333 |
| | SEQ ID Nos. 75 and 76) | | | | |
| 119 | TMEM30B (with primer | 0.87356 | 1 | 0.87755 | 0.49524 |
| | SEQ ID Nos: 77 and 78) | | | | |
| 46 | HOXA9_A | 0.98851 | 1 | 1 | 0.90476 |
| 12 | C10orf55 | 0.82266 | 0.88828 | 0.72959 | 0.95476 |
| 73 | MAX.chr17.73073682- | 0.9376 | 0.96703 | 0.94558 | 0.55238 |
| | 73073830 | | | | |
| 122 | TSPAN33 | 0.91461 | 0.91575 | 0.91837 | 0.35238 |
| 29 | FAM174B_B | 0.88998 | 0.90842 | 0.89116 | 0.52381 |
| 86 | MAX.chr5.60921627- | 0.85714 | 0.96154 | 0.86054 | 0.55714 |
| | 60921853 | | | | |
| 111 | SIX4_A | 0.92611 | 1 | 0.92177 | 0.40952 |
| 49 | KCNQ4_A | 0.84072 | 0.95971 | 0.88435 | 0.58571 |
| 32 | FOXP1 | 0.77833 | 0.85348 | 0.79592 | 0.54286 |
| 105 | OXT | 0.82594 | 0.9304 | 0.86054 | 0.62381 |
| 15 | C2orf82 | 0.88095 | 0.94505 | 0.87075 | 0.61429 |
| 117 | TAL1 (with primer SEQ | 0.87356 | 0.89377 | 0.89626 | 0.63571 |
| | ID Nos: 71 and 72) | | | | |
| 117 | TAL1 (with primer SEQ | 0.82266 | 0.91209 | 0.86735 | 0.64524 |
| | ID Nos: 73 and 74) | | | | |
| 61 | MAX.chr10.22541869- | 0.87521 | 0.91209 | 0.90136 | 0.65238 |
| | 22541953 | | | | |
| 99 | MIR155HG (with primer | 0.91954 | 0.92674 | 0.92177 | 0.62857 |
| | SEQ ID Nos: 53 and 54) | | | | |
| 99 | MIR155HG (with primer | 0.92447 | 0.94139 | 0.91837 | 0.4619 |
| | SEQ ID Nos: 55 and 56) | | | | |
| 85 | MAX.chr5.42992655- | 0.92939 | 0.98535 | 0.93878 | 0.61905 |
| | 42992768 | | | | |
| 97 | ME3 | 0.84401 | 0.92308 | 0.85374 | 0.38571 |
| 45 | HOPX | 0.85468 | 0.88095 | 0.85034 | 0.50952 |
| 21 | CLIC5 | 0.7619 | 0.79121 | 0.77211 | 0.48095 |
| 66 | MAX.chr13.29106812- | 0.78982 | 0.8022 | 0.80612 | 0.44762 |
| | 29106917 | | | | |
| 91 | MAX.chr6.29521537- | 0.97865 | 1 | 0.97619 | 0.53571 |
| | 29521696 | | | | |
| 16 | FOXL2NB | 0.90969 | 1 | 0.95238 | 0.6619 |
| 136 | FLJ22536_A | 0.95567 | 0.99267 | 0.95578 | 0.64762 |
| 138 | GALNT3_A | 0.82759 | 1 | 0.65646 | 0.99048 |
| 141 | ITPKA | 0.68309 | 1 | 0.59864 | 0.69524 |
| 144 | LMX1B | 0.76026 | 0.99267 | 0.59184 | 0.94286 |
| 146 | MAX.chr1.110627096- | 0.85057 | 0.85714 | 0.84354 | 0.53571 |
| | 110627364 | | | | |
| 151 | MAX.chr2.71116033- | 0.95895 | 1 | 0.93878 | 0.7619 |
| | 71116122 | | | | |
| 152 | MAX.chr20.21080958- | 0.94089 | 0.9707 | 0.96939 | 0.82857 |
| | 21081038 | | | | |
| 153 | MAX.chr7.155259597- | 1 | 1 | 1 | 0.46667 |
| | 155259763 (with primer | | | | |
| | SEQ ID Nos: 47 and 48) | | | | |
| 153 | MAX.chr7.155259597- | 0.99343 | 1 | 0.9932 | 0.58571 |
| | 155259763 (with primer | | | | |
| | SEQ ID Nos: 49 and 50) | | | | |
| 156 | NFATC2_A | 0.74713 | 0.97253 | 0.71429 | 0.7619 |
| 157 | OCA2 | 0.84893 | 1 | 0.78912 | 0.92857 |
| 158 | RNF207_A | 0.8046 | 0.96337 | 0.65646 | 0.98571 |
| 159 | RUNX2 | 0.86371 | 0.95604 | 0.87075 | 0.61429 |
| 162 | STX16 | 0.94253 | 1 | 0.88095 | 1 |

TABLE 5A
AUC—Area under the Curve (95% CI) & Median (IQR)
Marker/BTACT levels in Buffy samples

| | | | | | Cancer vs. Normal/ | | | Buffy Samples | | |
|---|---|---|---|---|---|---|---|---|---|---|
| DMR | | Cancer vs. Normal | | | Nevus | | | | 25th | 75th |
| No. | MDM | AUC | 95% CI | | AUC | 95% CI | | median | % | % |
| 46 | HOXA9_A | 0.99 | 0.97 | 1.01 | 0.97 | 0.95 | 1.09 | 0.001 | 0 | 0 |
| 16 | FOXL2NB | 0.97 | 0.94 | 1.00 | 0.96 | 0.93 | 1.00 | 0.001 | 0 | 9 |
| 138 | GALNT3_A | 0.96 | 0.92 | 1.00 | 0.75 | 0.66 | 0.84 | 0.000 | 0 | 9 |
| 152 | MAX.chr20.21080958- | 0.96 | 0.92 | 1.00 | 0.96 | 0.91 | 1.00 | 0.001 | 0 | 0 |
| | 21081038 | | | | | | | | | |
| 136 | FLJ22536_A | 0.95 | 0.91 | 0.99 | 0.93 | 0.87 | 0.98 | 0.003 | 0 | 0 |
| 146 | MAX.chr1.110627096- | 0.95 | 0.89 | 1.00 | 0.91 | 0.85 | 0.97 | 0.000 | 0 | 0 |
| | 110627364 (v2) | | | | | | | | | |
| 73 | MAX.chr17.73073682- | 0.95 | 0.91 | 1.00 | 0.95 | 0.90 | 1.00 | 0.000 | 0 | 0 |
| | 73073830 | | | | | | | | | |
| 117 | TAL1 (with primer | 0.94 | 0.89 | 0.99 | 0.93 | 0.88 | 0.98 | 0.004 | 0 | 0.01 |
| | SEQ ID Nos: 73 and 74) | | | | | | | | | |
| 151 | MAX.chr2.71116033- | 0.94 | 0.89 | 0.99 | 0.90 | 0.84 | 0.96 | 0.002 | 0 | 0.01 |
| | 71116122 | | | | | | | | | |
| 153 | MAX.chr7.155259597- | 0.94 | 0.88 | 0.99 | 0.93 | 0.87 | 0.98 | 0.000 | 0 | 0 |
| | 155259763 | | | | | | | | | |
| 156 | NFATC2_A | 0.94 | 0.88 | 0.99 | 0.83 | 0.75 | 0.90 | 0.000 | 0 | 0 |
| 158 | RNF207_A | 0.94 | 0.89 | 0.99 | 0.81 | 0.73 | 0.89 | 0.000 | 0 | 0 |
| 157 | OCA2 | 0.93 | 0.87 | 0.99 | 0.85 | 0.78 | 0.92 | 0.003 | 0 | 0 |
| 162 | STX16 | 0.93 | 0.87 | 0.99 | 0.84 | 0.76 | 0.92 | 0.002 | 0 | 0 |
| 86 | MAX.chr5.60921627- | 0.92 | 0.86 | 0.98 | 0.89 | 0.82 | 0.95 | 0.005 | 0 | 0.01 |
| | 60921853 | | | | | | | | | |
| 141 | ITPKA | 0.92 | 0.87 | 0.98 | 0.81 | 0.72 | 0.89 | 0.000 | 0 | 0 |
| 144 | LMX1B | 0.91 | 0.85 | 0.97 | 0.82 | 0.74 | 0.90 | 0.000 | 0 | 0 |
| 91 | MAX.chr6.29521537- | 0.90 | 0.83 | 0.97 | 0.88 | 0.81 | 0.95 | 0.000 | 0 | 0 |
| | 29521696 | | | | | | | | | |
| 85 | MAX.chr5.42992655- | 0.89 | 0.82 | 0.96 | 0.88 | 0.81 | 0.95 | 0.001 | 0 | 0 |
| | 42992768 | | | | | | | | | |
| 99 | MIR155HG (with primer | 0.87 | 0.80 | 0.95 | 0.88 | 0.82 | 0.95 | 0.000 | 0 | 0 |
| | SEQ ID Nos: 53 and 54) | | | | | | | | | |
| 99 | MIR155HG (with primer | 0.87 | 0.79 | 0.95 | 0.84 | 0.76 | 0.92 | 0.000 | 0 | 0 |
| | SEQ ID Nos: 55 and 56) | | | | | | | | | |
| 159 | RUNX2 | 0.87 | 0.79 | 0.95 | 0.79 | 0.71 | 0.88 | 0.001 | 0 | 0 |
| 64 | MAX.chr10.62492690- | 0.84 | 0.74 | 0.93 | 0.77 | 0.69 | 0.86 | 0.005 | 0 | 0.01 |
| | 62492812 | | | | | | | | | |
| 12 | C10orf55 | 0.84 | 0.76 | 0.93 | 0.77 | 0.69 | 0.86 | 0.000 | 0 | 0 |
| 29 | FAM174BB | 0.83 | 0.75 | 0.92 | 0.83 | 0.75 | 0.91 | 0.001 | 0 | 0 |
| 122 | TSPAN33 | 0.82 | 0.73 | 0.91 | 0.80 | 0.72 | 0.88 | 0.000 | 0 | 0 |
| 15 | C2orf82 | 0.79 | 0.70 | 0.89 | 0.78 | 0.69 | 0.87 | 0.000 | 0 | 0 |
| 73 | MAX.chr17.73073682- | 0.78 | 0.69 | 0.88 | 0.79 | 0.70 | 0.88 | 0.004 | 0 | 0 |
| | 73073830 | | | | | | | | | |
| 117 | TAL1 (with primer | 0.77 | 0.66 | 0.87 | 0.80 | 0.71 | 0.88 | 0.001 | 0 | 0 |
| | SEQ ID Nos: 71 and 72) | | | | | | | | | |
| 119 | TMEM30B (with primer | 0.69 | 0.58 | 0.81 | 0.73 | 0.63 | 0.82 | 0.002 | 0 | 0 |
| | SEQ ID Nos. 75 and 76) | | | | | | | | | |
| 105 | OXT | 0.51 | 0.36 | 0.65 | 0.51 | 0.40 | 0.62 | 0.006 | 0 | 0.01 |
| 61 | MAX.chr10.22541869- | 0.41 | 0.29 | 0.52 | 0.43 | 0.31 | 0.54 | 0.000 | 0 | 0 |
| | 22541953 | | | | | | | | | |

TABLE 5B
Random Forest Modelling--All 32 MDMs
AUC = 0.98 (Error Rate 4.63%, 0/47 = 0% Normal/Nevus, 5/61 = 8.2% Cancer)
Sensitivity @ Specificity (90, 95, 97.5, 99, 100)

| Specificity | Normal/Nevus | Sensitivity | Normal | Nevus | Metastatic | Primary |
|---|---|---|---|---|---|---|
| 90% | 42/47 | 58/61 = 95.08% | 20/20 | 22/27 | 35/35 | 23/26 |
| 95% | 44/47 | 58/61 = 95.08% | 20/20 | 24/27 | 35/35 | 23/26 |
| 97.5% | 45/47 | 58/61 = 95.08% | 20/20 | 25/27 | 35/35 | 23/26 |
| 99% | 46/47 | 58/61 = 95.08% | 20/20 | 26/27 | 35/35 | 23/26 |
| 100% | 47/47 | 57/61 = 93.44% | 20/20 | 27/27 | 34/35 | 23/26 |

TABLE 5C
Random Forest Modelling--5 MDMs (MAX.chr20.21080958-21081038.
MAX.chr7.155259597-155259763 (using primer SEQ ID Nos. 47 and 48),
MAX.chr7.155259597-155259763 (using primer SEQ ID Nos. 49 and 50),
FOXL2NB and HOXA9_A)
AUC = 0.97 (Error Rate 5.56%, 1/47 = 2.1% Normal/Nevus, 5/61 = 8.2% Cancer)
Sensitivity @ Specificity (90. 95, 97.5. 99, 100)

| Specificity | Normal/Nevus | Sensitivity | Normal | Nevus | Metastatic | Primary |
|---|---|---|---|---|---|---|
| 90% | 42/47 | 59/61 = 96.72% | 18/20 | 24/27 | 35/35 | 24/26 |
| 95% | 44/47 | 57/61 = 93.44% | 19/20 | 25/27 | 34/35 | 23/26 |
| 97.5% | 45/47 | 56/61 = 91.80% | 19/20 | 26/27 | 33/35 | 23/26 |
| 99% | 46/47 | 56/61 = 91.80% | 20/20 | 26/27 | 33/35 | 23/26 |
| 100% | 47/47 | 55/61 = 90.16% | 20/20 | 27/27 | 33/35 | 22/26 |

| | Normal and Nevus | Primary | Metastatic |
|---|---|---|---|
| | (n = 47) | (n = 26) | (n = 35) |
| | Median methylation | Median methylation | Median methylation |
| MDMs | value (IQR) | value (IQR) | value (IQR) |
| HOXA9_A | −0.19 (−0.83, 0.56) | 3.08 (2.03, 3.40) | 4.43 (3.60, 5.53) |
| FOXL2NB | 0.0 (−0.45, 0.36) | 3.02 (1.68, 3.56) | 4.32 (3.61, 5.52) |
| chr7.155259614-81 | 0.08 (−0.60, 0.57) | 2.46 (1.68, 3.06) | 4.26 (2.96, 4.85) |
| chr7.155259700-746 | −0.1 (−0.54, 0.53) | 2.63 (2.22, 3.42) | 4.64 (3.49, 5.34) |
| chr20.21080958-1038 | 0.03 (−0.29, 0.36) | 2.75 (1.94, 3.38) | 3.49 (3.04, 4.56) |

TABLE 6

| | | Cancer | | Buffy | | |
|---|---|---|---|---|---|---|
| | | vs Buffy | | Samples | | |
| DMR No. | Gene Annotation | AUC | FC | median | 25^{th} % | 75^{th} % |
| 12 | C10orf55 | 0.87 | 157.28 | 0 | 0 | 0 |
| 15 | C2orf82 | 0.82 | 40.33 | 0 | 0 | 0 |
| 16 | FOXL2NB | 0.97 | 103.01 | 0.001 | 0 | 0 |
| 29 | FAM174B_B | 0.90 | 103.81 | 0.001 | 0 | 0 |
| 136 | FLJ22536_A | 0.98 | 47.52 | 0.003 | 0 | 0 |
| 138 | GALNT3_A | 0.98 | 62.01 | 0 | 0 | 0 |
| 46 | HOXA9_A | 0.97 | 71.30 | 0.001 | 0 | 0 |
| 141 | ITPKA | 0.96 | 65.57 | 0 | 0 | 0 |
| 144 | LMX1B | 0.95 | 89.00 | 0 | 0 | 0 |
| 146 | MAX.chr1.110627096- | 0.95 | 48.81 | 0 | 0 | 0 |
| | 110627364 | | | | | |
| 61 | MAX.chr10.22541869- | 0.75 | 1076.66 | 0 | 0 | 0 |
| | 22541953 | | | | | |
| 64 | MAX.chr10.62492690- | 0.92 | 7.48 | 0.005 | 0 | 0.01 |
| | 62492812 | | | | | |
| 73 | MAX.chr17.73073682- | 0.95 | 23.71 | 0.004 | 0 | 0 |
| | 73073830 | | | | | |
| 151 | MAX.chr2.71116033- | 0.97 | 40.38 | 0.002 | 0 | 0.01 |
| | 71116122 | | | | | |
| 152 | MAX.chr20.21080958- | 0.98 | 82.25 | 0.001 | 0 | 0 |
| | 21081038 | | | | | |
| 85 | MAX.chr5.42992655- | 0.93 | 114.46 | 0.001 | 0 | 0 |
| | 42992768 | | | | | |
| 86 | MAX.chr5.60921627- | 0.93 | 18.91 | 0.005 | 0 | 0.01 |
| | 60921853 | | | | | |
| 91 | MAX.chr6.29521537- | 0.87 | 81.07 | 0 | 0 | 0 |
| | 29521696 | | | | | |
| 153 | MAX.chr7.155259597- | 0.95 | 71.71 | 0 | 0 | 0 |
| | 155259763 (with primer | | | | | |
| | SEQ ID Nos: 47 and 48) | | | | | |
| 153 | MAX.chr7.155259597- | 0.96 | 65.69 | 0 | 0 | 0 |
| | 155259763 (with primer | | | | | |
| | SEQ ID Nos: 49 and 50) | | | | | |
| 99 | MIR155HG (with primer | 0.91 | 356.84 | 0 | 0 | 0 |
| | SEQ ID Nos: 53 and 54) | | | | | |
| 99 | MIR155HG (with primer | 0.89 | 234.33 | 0 | 0 | 0 |
| | SEQ ID Nos: 55 and 56) | | | | | |
| 156 | NFATC2_A | 0.98 | 66.97 | 0 | 0 | 0 |
| 157 | OCA2 | 0.97 | 41.89 | 0.003 | 0 | 0 |
| 105 | OXT | 0.86 | 7.31 | 0.006 | 0 | 0.01 |
| 158 | RNF207_A | 0.98 | 62.29 | 0 | 0 | 0 |
| 159 | RUNX2 | 0.89 | 44.93 | 0.001 | 0 | 0 |
| 162 | STX16 | 0.97 | 41.23 | 0.002 | 0 | 0 |
| 117 | TAL1 (with primer SEQ ID | 0.88 | 19.99 | 0.001 | 0 | 0 |
| | Nos: 71 and 72) | | | | | |
| 117 | TAL1 (with primer SEQ ID | 0.95 | 28.09 | 0.004 | 0 | 0.01 |
| | Nos: 73 and 74) | | | | | |
| 119 | TMEM30BV1 | 0.80 | 26.91 | 0.002 | 0 | 0 |
| 122 | TSPAN33 | 0.80 | 103.05 | 0 | 0 | 0 |

### Example II

### Materials and Methods

Whole Genome Bisulfite Sequencing (WGBS) is an NGS approach which differs from a usual RRBS method in that the entire human genome is sequenced. RRBS is an enrichment method which uses restriction endonucleases specific to CpG rich cut sites to generate fragments from transcriptional regulatory regions of the genome. These CpG islands have been shown to exhibit differential or altered methylation profiles in numerous clinical states, most notably cancer. The benefit of RRBS is that is reduces the size of the genome to 1-2% of the total 3.2 billion nucleotides, substantially reducing cost while capturing the majority of promoters and CpG island. The downside is that is does miss a potentially substantial number of regulatory regions which either are CpG rich but do not contain the endonuclease binding sequence or those that are not part of a CpG island. Additionally, the results are somewhat biased by the performance and efficiency of the enzyme which can differ depending on the sample. WGBS uses physical shearing to chop the genome into 200-300 bp fragments which are amenable to NGS. The upside is that every fragment is theoretically sequenced, irrespective of CpG content, leading to potentially larger numbers of tumor specific biomarkers. The disadvantage is that most of the genome is not regulatory or suitable for biomarker discovery and so will be discarded. WGBS is also more costly to perform at a comparable depth of vertical coverage. The advent of higher capacity sequencers and flow cells, and the advance of NGS technology in general, has mitigated the cost to some degree—although it still remains less affordable than RRBS or other enrichment sequencing protocols.

Samples were identical to those used in the RRBS study to allow for direct comparison. Tissue and blood was obtained from Mayo Clinic biospecimen repositories with institutional IRB oversight. Samples were chosen with strict adherence to subject research authorization and inclusion/exclusion criteria. Cancers consisted of 21 metastatic melanomas. Controls included 15 non-neoplastic skin epidermis samples, 16 benign melanocytic nevi, and 36 whole blood derived leukocytes. Tissues were macro-dissected and histology reviewed by an expert pathologist. Samples were age matched, randomized, and blinded. DNA was purified using the QIAamp DNA Tissue Mini kit and QIAamp DNA Blood Mini kit (Qiagen, Valencia Calif.), respectively. DNA was re-purified with AMPure XP beads (Beckman-Coulter, Brea Calif.) and quantified by PicoGreen (Thermo-Fisher, Waltham Mass.). DNA integrity was assessed using qPCR.

Sequencing libraries were prepared following the method of Ulrich et al (Nature Protocols. 10, 475-483 (2015)) with modifications. Briefly 100 ng of DNA was sheared on a Covaris ultrasonicator to a 200 bp target size. Fragments >600 bp were size selected away using 0.6× ampure beads (Beckman). The supernatant was then brought up to 1.4× to purify the remaining fragments with a lower limit of 100 bp. Ends were repaired with the End-It DNA end-repair kit (Epicentre) and purified with 1.4× Ampure beads, followed by a-tailing using Klenow (3′-5′ exo-(NEB) and a subsequent 1.4× purification. Methylated adapters (NEXTflex Bisulfite-Seq Barcodes—Bioo Scientific) were ligated with T4 DNA Ligase (NEB), bisulfite treated twice with the Epitect 96 Kit (Qiagen), and bead purified. Libraries were then tested by SYBR Green qPCR to determine optimal enrichment cycles—and then enriched by either 10,13,16, or 18 cycles using the KAPA HiFi HotStart Uracil+ReadyMix Kit (Kapa Biosystems). Enriched libraries were bead purified at a 1:1 ratio, indexed together in groups of 24, re-purified, and quantified on the Bioanalyzer 2100 (Agilent) and using qPCR (Kapa). Concentrations ranged from 5-80 nM.

Paired end 150 cycle sequencing was performed on the Nova-Seq using 1 S4 flow cell per 24 samples (Illumina). Reads were processed by Illumina pipeline modules for image analysis and base calling. Secondary analysis was performed using a WGBS modified version of SAAP-RRBS, a Mayo developed bioinformatics suite. Briefly, reads were cleaned-up using Trim-Galore and aligned to the GRCh37/hg19 reference genome build with BSMAP. Methylation ratios were determined by calculating C/(C+T) or conversely, G/(G+A) for reads mapping to reverse strand, for CpGs with coverage ≥5× and base quality score ≥20.

A proprietary DMR identification pipeline and regression package was used to derive DMRs based on average methylation values of the CpG. The difference in average methylation percentage was compared between malignant melanoma cases, tissue controls and buffy coat controls; a tiled reading frame within 100 base pairs of each mapped CpG was used to identify DMRs where control methylation was <5%; DMRs were only analyzed if the total depth of coverage was 5 reads per subject on average and the variance across subgroups was >0.

Following regression, DMRs were ranked by p-value, area under the receiver operating characteristic curve (AUC) and fold-change difference between cases and all controls. No adjustments for false discovery were made during this phase as independent validation was planned a priori.

Individual CpGs were ranked by hypermethylation ratio, namely the number of methylated cytosines at a given locus over the total cytosine count at that site. For cases, the ratios were required to be ≥0.20 (20%); for tissue controls, ≤0.05 (5%) tissue vs tissue analysis; ≥0.30 (30%) tissue vs buffy coat; for buffy coat controls, ≤0.01 (1%). CpGs which did not meet these criteria were discarded. Subsequently, candidate CpGs were binned by genomic location into DMRs (differentially methylated regions) ranging from approximately 30-300 bp with a minimum cut-off of 5 CpGs per region. DMRs with excessively high CpG density (>30%) were excluded to avoid GC-related amplification problems in the validation phase. For each candidate region, a 2-D matrix was created which compared individual CpGs in a sample-to-sample fashion for both cases and controls. These CpG matrices were then compared back to the reference sequence to assess whether genomically contiguous methylation sites had been discarded during the initial filtering. From this subset of regions, final selections required coordinated and contiguous hypermethylation (in cases) of individual CpGs across the DMR sequence on a per sample level. Conversely, control samples had to have at least 10-fold less methylation than cases and the CpG pattern had to be more random and less coordinated.

### Results

A modified WGBS methodology of sample preparation, sequencing, in combination with proprietary analyses pipelines and filters (outlined in Methods) was used to identify and narrow differentially methylated regions (DMRs) to those which would pinpoint these skin cancers and excel in a clinical testing environment. The WGBS DMRs showed substantial (roughly 50%) overlap with RRBS DMRs and the performance between the two studies correlated very well. However, there were many high performing biomarkers which were not seen with in the RRBS data. From the tissue-to-tissue analysis, 48 hypermethylated malignant melanomas (MM) DMRs were identified (Table 7A). All had AUCs>0.90 and FC ratios >10 (Table 7B). The tissue to leukocyte (buffy coat) analysis yielded 111 hypermethylated MM+epidermis tissue DMRs with less than 1% noise in WBCs (Table 8A). AUCs for all 111 were >0.95 and FC ratios >50 (Table 8B). The DMRs included in these tables were ones that did not appear in the RRBS study.

TABLE 7A

| | | | DMR Start-End Positions |
|---|---|---|---|
| DMR No. | Gene Annotation | Chromosome No. | (GRCh37/hg19) |
| 163 | AGRN | 1 | 969279-969321 |
| 164 | BMP8B | 1 | 40236184-40236211 |
| 165 | C17orf46_B | 17 | 43339390-43339782 |
| 166 | C17orf57_B | 17 | 45500815-45500840 |
| 167 | C2CD4A | 15 | 62359116-62359191 |
| 168 | C2CD4D_B | 1 | 151811304-151811332 |
| 169 | CDYL | 6 | 4775223-4775857 |
| 170 | CMAH | 6.00E+00 | 25137865-25137971 |
| 171 | DENND2D | 1 | 111747303-111747428 |
| 172 | DLEU2 | 13 | 50701729-50701773 |
| 173 | DSCR6 | 21 | 38378747-38378780 |
| 174 | FLJ22536_B | 6 | 21666257-21667030 |
| 175 | FLJ45983 | 10 | 8097151-8097279 |
| 176 | FOXF2 | 6 | 1393820-1393871 |
| 177 | GALNT3_B | 2 | 166650395-166650909 |
| 178 | HCG4P6 | 6 | 29895017-29895100 |
| 179 | HOXA9_B | 7 | 27205761-27205801 |
| 180 | KIFC2 | 8 | 145697900-145697946 |
| 181 | LDLRAD2 | 1 | 22140884-22141031 |
| 182 | LY75 | 2 | 160760468-160760605 |
| 183 | LYL1 | 19 | 13210266-13210316 |
| 184 | LYN | 8 | 56791976-56792040 |
| 185 | MAPK13 | 6 | 36098567-36098815 |
| 186 | MAX.chr1.1072486-1072508 | 1 | 1072486-1072508 |
| 187 | MAX.chr1.32237693-32237785 | 1 | 32237693-32237785 |
| 188 | MAX.chr10.62492374-62492793 | 10 | 62492374-62492793 |
| 189 | MAX.chr11.14926535-14926715 | 11 | 14926535-14926715 |
| 190 | MAX.chr16.54970444-54970469 | 16 | 54970444-54970469 |
| 191 | MAX.chr19.16439332-16439390 | 19 | 16439332-16439390 |
| 192 | MAX.chr20.21080670-21081280 | 20 | 21080670-21081280 |
| 193 | MAX.chr4.113432264-113432298 | 4 | 113432264-113432298 |
| 194 | MAX.chr7.129425668-129425719 | 7 | 129425668-129425719 |
| 195 | MAX.chr8.82543163-82543213 | 8 | 82543163-82543213 |
| 196 | PARP15 | 3 | 122296541-122296587 |
| 197 | PRKAG2 | 7 | 151329831-151329982 |
| 198 | PROC_A | 2 | 128173619-128173670 |
| 199 | PROM1_B | 4 | 16085259-16085484 |
| 200 | PTGER4_A | 5 | 40681512-40681876 |
| 201 | PTP4A3 | 8 | 142428210-142428265 |
| 202 | SDCCAG8 | 1 | 243646466-243646488 |
| 203 | SH3PXD2A | 10 | 105453184-105453230 |
| 204 | SLC2A2 | 3 | 170746270-170746292 |
| 205 | SLC35D3 | 6 | 137241799-137241883 |
| 206 | TBR1 | 2 | 162271760-162271819 |
| 207 | TBX2 | 17 | 59481777-59481811 |
| 208 | TCP11 | 6 | 35108849-35108977 |
| 209 | TFAP2A | 6 | 10419530-10419646 |
| 210 | TRIM73 | 7 | 75028743-75028905 |

TABLE 7B

| | | Area Under | | |
|---|---|---|---|---|
| DMR No. | Gene Annotation | Curve | Fold-Change | p-value |
| 163 | AGRN | 1 | 33.01 | 0.006078 |
| 164 | BMP8B | 0.9643 | 19.48 | 0.005893 |
| 165 | C17orf46_B | 0.9333 | 25.6 | 0.004365 |
| 166 | C17orf57_B | 0.9464 | 15.15 | 0.003556 |
| 167 | C2CD4A | 0.981 | 35.75 | 0.004804 |
| 168 | C2CD4D_B | 1 | 24.7 | 0.004156 |
| 169 | CDYL | 1 | 48.54 | 0.00356 |
| 170 | CMAH | 1 | 34.77 | 2.81E−05 |
| 171 | DENND2D | 0.9333 | 17.63 | 0.004753 |
| 172 | DLEU2 | 1 | 44.81 | 0.002166 |
| 173 | DSCR6 | 0.9567 | 56.06 | 0.008127 |
| 174 | FLJ22536_B | 0.9619 | 54.14 | 0.008343 |
| 175 | FLJ45983 | 0.9333 | 14.11 | 0.009994 |
| 176 | FOXF2 | 0.9167 | 17.63 | 0.003168 |
| 177 | GALNT3_B | 1 | 112.6 | 0.002757 |
| 178 | HCG4P6 | 0.9 | 34.97 | 0.008641 |
| 179 | HOXA9_B | 1 | 34.46 | 0.001253 |
| 180 | KIFC2 | 0.9833 | 92.67 | 0.004278 |
| 181 | LDLRAD2 | 0.9286 | 39.68 | 0.003913 |
| 182 | LY75 | 0.9125 | 11.68 | 0.000547 |
| 183 | LYL1 | 0.9898 | 14.97 | 0.00264 |
| 184 | LYN | 0.9619 | 24.06 | 0.006377 |
| 185 | MAPK13 | 1 | 69.67 | 0.001806 |
| 186 | MAX.chr1.1072486-1072508 | 0.9082 | 51.23 | 0.001488 |
| 187 | MAX.chr1.32237693-32237785 | 0.9833 | 30.18 | 0.004089 |
| 188 | MAX.chr10.62492374-62492793 | 0.9583 | 11.59 | 0.001119 |
| 189 | MAX.chr11.14926535-14926715 | 0.9833 | 33.21 | 0.001753 |
| 190 | MAX.chr16.54970444-54970469 | 0.9083 | 25.38 | 0.009217 |
| 191 | MAX.chr19.16439332-16439390 | 0.981 | 25.69 | 0.003778 |
| 192 | MAX.chr20.21080670-21081280 | 0.9125 | 17.31 | 0.003164 |
| 193 | MAX.chr4.113432264-113432298 | 0.9541 | 35.56 | 0.001857 |
| 194 | MAX.chr7.129425668-129425719 | 0.9429 | 21.28 | 0.006163 |
| 195 | MAX.chr8.82543163-82543213 | 0.9238 | 23.35 | 0.007549 |
| 196 | PARP15 | 1 | 50.61 | 0.002434 |
| 197 | PRKAG2 | 0.9667 | 28.21 | 0.001717 |
| 198 | PROC_A | 0.9476 | 28.81 | 0.001883 |
| 199 | PROM1_B | 0.9375 | 40.17 | 0.004806 |
| 200 | PTGER4_A | 1 | 33 | 0.002816 |
| 201 | PTP4A3 | 0.9429 | 20.28 | 0.01 |
| 202 | SDCCAG8 | 0.9083 | 27.26 | 0.005528 |
| 203 | SH3PXD2A | 0.9241 | 53.51 | 0.009754 |
| 204 | SLC2A2 | 0.95 | 65.19 | 0.005763 |
| 205 | SLC35D3 | 0.9292 | 11.24 | 0.003726 |
| 206 | TBR1 | 0.9042 | 18.6 | 0.003899 |
| 207 | TBX2 | 0.9333 | 22.17 | 0.007544 |
| 208 | TCP11 | 0.9898 | 58.65 | 0.001706 |
| 209 | TFAP2A | 0.9087 | 38.54 | 0.007251 |
| 210 | TRIM73 | 1 | 37.94 | 0.005082 |

TABLE 8A

| | | | DMR Start-End Positions |
|---|---|---|---|
| DMR No. | Gene Annotation | Chromosome No. | (GRCh37/hg19) |
| 211 | ABHD15 | 17 | 27893358-27893434 |
| 212 | ACAP1 | 17 | 7240058-7240106 |
| 213 | ACTB | 7 | 5571593-5571712 |
| 214 | ADRBK1 | 11 | 67035439-67036211 |
| 215 | ANO9 | 11 | 441940-442058 |
| 216 | ATP6V1B1 | 2 | 71192413-71192452 |
| 217 | BEST4 | 1 | 45252148-45252191 |
| 218 | C1orf177 | 1 | 55266723-55266832 |
| 219 | CCDC140 | 2 | 223167358-223167385 |
| 220 | CCND3 | 6 | 41908118-41908158 |
| 221 | CD69 | 12 | 9917353-9917467 |
| 222 | CDC42EP1 | 22 | 37962519-37962558 |
| 223 | CHRM1 | 11 | 62693755-62693782 |
| 224 | CSK | 15 | 75069519-75069619 |
| 225 | CYTH1 | 17 | 76771324-76771584 |
| 226 | DDN | 12 | 49391122-49391209 |
| 227 | DEDD2 | 19 | 42703648-42703891 |
| 228 | DGKZ | 11 | 46367778-46367847 |
| 229 | DHH | 12 | 49483661-49483738 |
| 230 | DLG5 | 10 | 79633792-79633845 |
| 231 | DNM2 | 19 | 10870312-10870427 |
| 232 | DNMT3A | 2 | 25499764-25499804 |
| 233 | DUSP2 | 2 | 96812221-96812268 |
| 234 | ESRRG | 1 | 217308088-217308118 |
| 235 | FBRSL1 | 12 | 133065476-133065776 |
| 236 | FOXP1 | 3 | 71630818-71630894 |
| 237 | FOXP4_A | 6 | 41515929-41515972 |
| 238 | FOXP4_B | 6 | 41528310-41528503 |
| 239 | FRZB | 2 | 183731563-183731600 |
| 240 | GALNT3_C | 2 | 166650352-166650402 |
| 241 | GATA2 | 3 | 128211626-128211681 |
| 242 | GJB2 | 13 | 20767909-20768079 |
| 243 | GNG7 | 19 | 2620764-2620859 |
| 244 | GP5_A | 3 | 194117873-194117897 |
| 245 | GP5_B | 3 | 194118511-194118559 |
| 246 | GP5_C | 3 | 194118799-194118838 |
| 247 | GPR132 | 14 | 105527858-105527909 |
| 248 | GPR135_B | 14 | 59931142-59931177 |
| 249 | HAAO | 2 | 43019992-43020017 |
| 250 | HOXB5_C | 17 | 46671011-46671084 |
| 251 | HOXB5_D | 17 | 46673976-46674007 |
| 252 | HSF5 | 17 | 56565263-56565284 |
| 253 | ICAM2 | 17 | 62097597-62097723 |
| 254 | IFFO2 | 1 | 19250393-19250485 |
| 255 | KCNH3 | 12 | 49943015-49943046 |
| 256 | KCNQ4_B | 1 | 41284305-41284411 |
| 257 | KIAA0182 | 16 | 85649545-85649580 |
| 258 | KLHDC7B | 22 | 50987206-50987271 |
| 259 | KREMEN1_B | 22 | 29467754-29467795 |
| 260 | LHFPL2 | 5 | 77805840-77805876 |
| 261 | LIMD1 | 3 | 45706043-45706236 |
| 262 | LOC440925 | 2 | 171570298-171570337 |
| 263 | MAP3K5 | 6 | 137112514-137112610 |
| 264 | MAX.chr1.203256204-203256316 | 1 | 203256204-203256316 |
| 265 | MAX.chr1.203258714-203258909 | 1 | 203258714-203258909 |
| 266 | MAX.chr1.22366401-22366546 | 1 | 22366401-22366546 |
| 267 | MAX.chr1.54941184-54941237 | 1 | 54941184-54941237 |
| 268 | MAX.chr11.14927004-14927275 | 11 | 14927004-14927275 |
| 269 | MAX.chr12.30976208-30976253 | 12 | 30976208-30976253 |
| 270 | MAX.chr15.31556237-31556534 | 15 | 31556237-31556534 |
| 271 | MAX.chr19.41834559-41834634 | 19 | 41834559-41834634 |
| 272 | MAX.chr19.50003691-50003720 | 19 | 50003691-50003720 |
| 273 | MAX.chr2.69135655-69135781 | 2 | 69135655-69135781 |
| 274 | MAX.chr21.37670549-37670625 | 21 | 37670549-37670625 |
| 275 | MAX.chr22.36848164-36848205 | 22 | 36848164-36848205 |
| 276 | MAX.chr4.186050036-186050085 | 4 | 186050036-186050085 |
| 277 | MAX.chr5.42992599-42992772 | 5 | 42992599-42992772 |
| 278 | MAX.chr8.101822019-101822077 | 8 | 101822019-101822077 |
| 279 | MAX.chr8.80695857-80695919 | 8 | 80695857-80695919 |
| 280 | MAX.chr8.80803443-80803495 | 8 | 80803443-80803495 |
| 281 | MAX.chr9.139595896-139596026 | 9 | 139595896-139596026 |
| 282 | MORN3 | 12 | 122096796-122096850 |
| 283 | MPZ_B | 1 | 161275414-161275553 |
| 284 | NDRG4 | 16 | 58535208-58535288 |
| 285 | NFATC2_B | 20 | 50158872-50159008 |
| 286 | OTX1 | 2 | 63283968-63284002 |
| 287 | PAMR1 | 11 | 35547114-35547153 |
| 288 | PDLIM2_A | 8 | 22437870-22438005 |
| 289 | PDLIM2_B | 8 | 22438128-22438191 |
| 290 | PHF20 | 20 | 34356073-34356200 |
| 291 | PPFIA4 | 1 | 203044814-203044863 |
| 292 | PROC_B | 2 | 128173738-128173906 |
| 293 | PRR15 | 7 | 29603319-29603434 |
| 294 | PSTPIP1 | 15 | 77287762-77287907 |
| 295 | PTGER4_B | 5 | 40681167-40681229 |
| 296 | PTK2B | 8 | 27183885-27184171 |
| 297 | PTPN6 | 12 | 7060368-7060475 |
| 298 | RGS3 | 9 | 116342944-116343115 |
| 299 | RIN2 | 20 | 19917988-19918066 |
| 300 | RNF207_B | 1 | 6265952-6265974 |
| 301 | RNF207_C | 1 | 6266219-6266258 |
| 302 | RNF220_B | 1 | 44874701-44874823 |
| 303 | RNF44 | 5 | 175960664-175960896 |
| 304 | RPS6KA1 | 1 | 26868823-26868917 |
| 305 | RPTOR | 17 | 78638920-78639074 |
| 306 | SELPLG | 12 | 109029388-109029607 |
| 307 | SGK1 | 6 | 134561914-134562045 |
| 308 | SLC2A1 | 1 | 43397754-43398090 |
| 309 | SMARCA4 | 19 | 11159902-11160083 |
| 310 | SPTBN1 | 2 | 54785828-54785886 |
| 311 | SYN3 | 22 | 33023163-33023242 |
| 312 | SYTL1 | 1 | 27669462-27669641 |
| 313 | TFAP2E | 1 | 36042961-36043015 |
| 314 | TFEB | 6 | 41675581-41675635 |
| 315 | THAP4 | 2 | 242549862-242549967 |
| 316 | TOX | 8 | 60030355-60030405 |
| 317 | TRABD | 22 | 50629861-50630029 |
| 318 | TRH | 3 | 129693636-129693699 |
| 319 | TXNRD2 | 22 | 19879091-19879295 |
| 320 | UST | 6 | 149082648-149082945 |
| 321 | WDR66 | 12 | 122356325-122356391 |

TABLE 8B

| | | Area Under | | |
|---|---|---|---|---|
| DMR No. | Gene Annotation | Curve | Fold-Change | p-value |
| 211 | ABHD15 | 0.9667 | 7.88E+08 | 0.995 |
| 212 | ACAP1 | 1 | 1.82E+09 | 0.9929 |
| 213 | ACTB | 0.9667 | 1.51E+09 | 0.9954 |
| 214 | ADRBK1 | 1 | 198.6 | 0.0004594 |
| 215 | ANO9 | 1 | 1.27E+09 | 0.9951 |
| 216 | ATP6V1B1 | 0.9667 | 8.37E+08 | 0.9949 |
| 217 | BEST4 | 0.95 | 78.09 | 0.007705 |
| 218 | C1orf177 | 0.9643 | 7E+08 | 0.9949 |
| 219 | CCDC140 | 1 | 199.8 | 0.009637 |
| 220 | CCND3 | 1 | 8.02E+08 | 0.9956 |
| 221 | CD69 | 0.9533 | 152.9 | 0.00634 |
| 222 | CDC42EP1 | 1 | 251.6 | 9.06E−05 |
| 223 | CHRM1 | 0.9643 | 6.03E+08 | 0.9949 |
| 224 | CSK | 0.9667 | 4.6E+08 | 0.9932 |
| 225 | CYTH1 | 1 | 2.66E+09 | 0.9936 |
| 226 | DDN | 0.9667 | 1.15E+09 | 0.9952 |
| 227 | DEDD2 | 1 | 608.8 | 0.0006947 |
| 228 | DGKZ | 1 | 1.90E+09 | 0.994 |
| 229 | DHH | 1 | 5.72E+08 | 0.9937 |
| 230 | DLG5 | 0.9733 | 73.42 | 0.002512 |
| 231 | DNM2 | 1 | 104.5 | 0.001014 |
| 232 | DNMT3A | 1 | 4.06E+09 | 0.9948 |
| 233 | DUSP2 | 1 | 172.6 | 0.006556 |
| 234 | ESRRG | 0.9667 | 2.04E+08 | 0.9944 |
| 235 | FBRSL1 | 1 | 136.1 | 0.007791 |
| 236 | FOXP1 | 0.9667 | 2.16E+08 | 0.9933 |
| 237 | FOXP4_A | 0.9519 | 201.6 | 0.00375 |
| 238 | FOXP4_B | 1 | 3.88E+08 | 0.9941 |
| 239 | FRZB | 0.9667 | 1.38E+09 | 0.9949 |
| 240 | GALNT3_C | 0.9643 | 1.19E+09 | 0.9948 |
| 241 | GATA2 | 0.9667 | 7.78E+08 | 0.9948 |
| 242 | GJB2 | 1 | 1.51E+09 | 0.9952 |
| 243 | GNG7 | 0.9667 | 1.87E+09 | 0.9942 |
| 244 | GP5_A | 1 | 2.13E+09 | 0.9934 |
| 245 | GP5_B | 0.9567 | 197.3 | 0.005652 |
| 246 | GP5_C | 1 | 130.6 | 0.00432 |
| 247 | GPR132 | 1 | 1.17E+09 | 0.9941 |
| 248 | GPR135_B | 0.9643 | 6.89E+08 | 0.9958 |
| 249 | HAAO | 0.9615 | 2.6E+08 | 0.9935 |
| 250 | HOXB5_C | 0.96 | 207.2 | 0.009996 |
| 251 | HOXB5_D | 0.9933 | 222.6 | 0.004066 |
| 252 | HSF5 | 1 | 615.6 | 0.0015 |
| 253 | ICAM2 | 0.9667 | 6.27E+08 | 0.9942 |
| 254 | IFFO2 | 0.9567 | 82.21 | 0.008566 |
| 255 | KCNH3 | 1 | 116.9 | 0.000725 |
| 256 | KCNQ4_B | 0.9643 | 8.78E+08 | 0.9953 |
| 257 | KIAA0182 | 0.9556 | 112.2 | 0.008084 |
| 258 | KLHDC7B | 0.9667 | 5.54E+08 | 0.9941 |
| 259 | KREMEN1_B | 0.9667 | 8.69E+08 | 0.9939 |
| 260 | LHFPL2 | 0.95 | 105.9 | 0.005347 |
| 261 | LIMD1 | 1 | 278.2 | 0.008128 |
| 262 | LOC440925 | 1 | 1.31E+09 | 0.9948 |
| 263 | MAP3K5 | 0.9667 | 4.22E+08 | 0.9931 |
| 264 | MAX.chr1.203256204-203256316 | 0.9933 | 107.5 | 0.001409 |
| 265 | MAX.chr1.203258714-203258909 | 1 | 304.1 | 0.0091 |
| 266 | MAX.chr1.22366401-22366546 | 1 | 1.40E+09 | 0.9954 |
| 267 | MAX.chr1.54941184-54941237 | 1 | 4.76E+08 | 0.9928 |
| 268 | MAX.chr11.14927004-14927275 | 1 | 434.4 | 0.007614 |
| 269 | MAX.chr12.30976208-30976253 | 1 | 282.8 | 0.007978 |
| 270 | MAX.chr15.31556237-31556534 | 1 | 152.8 | 0.006391 |
| 271 | MAX.chr19.41834559-41834634 | 1 | 233.6 | 0.009335 |
| 272 | MAX.chr19.50003691-50003720 | 0.9667 | 1.64E+09 | 0.9955 |
| 273 | MAX.chr2.69135655-69135781 | 1 | 99.2 | 0.008221 |
| 274 | MAX.chr21.37670549-37670625 | 1 | 1.21E+09 | 0.9955 |
| 275 | MAX.chr22.36848164-36848205 | 0.9867 | 137.2 | 0.005796 |
| 276 | MAX.chr4.186050036-186050085 | 1 | 68.76 | 0.0057 |
| 277 | MAX.chr5.42992599-42992772 | 0.9667 | 2.6E+08 | 0.9937 |
| 278 | MAX.chr8.101822019-101822077 | 0.9567 | 312.5 | 0.008165 |
| 279 | MAX.chr8.80695857-80695919 | 1 | 4.45E+08 | 0.9935 |
| 280 | MAX.chr8.80803443-80803495 | 1 | 2.32E+08 | 0.9938 |
| 281 | MAX.chr9.139595896-139596026 | 0.96 | 133.8 | 0.001154 |
| 282 | MORN3 | 1 | 9.83E+08 | 0.9946 |
| 283 | MPZ_B | 1 | 8.55E+08 | 0.9943 |
| 284 | NDRG4 | 0.9667 | 4.43E+08 | 0.9929 |
| 285 | NFATC2_B | 1 | 2.06E+09 | 0.9937 |
| 286 | OTX1 | 0.9923 | 258.6 | 0.001903 |
| 287 | PAMR1 | 0.9667 | 6.26E+08 | 0.9944 |
| 288 | PDLIM2_A | 1 | 1.59E+09 | 0.9951 |
| 289 | PDLIM2_B | 1 | 389.6 | 0.008777 |
| 290 | PHF20 | 0.9643 | 1.87E+08 | 0.9943 |
| 291 | PPFIA4 | 1 | 1.66E+09 | 0.9945 |
| 292 | PROC_B | 0.95 | 188.7 | 0.002602 |
| 293 | PRR15 | 0.9567 | 122.9 | 0.002215 |
| 294 | PSTPIP1 | 1 | 308.6 | 0.008096 |
| 295 | PTGER4_B | 0.9643 | 9.54E+08 | 0.9953 |
| 296 | PTK2B | 1 | 242.6 | 0.003993 |
| 297 | PTPN6 | 1 | 4.84E+09 | 0.995 |
| 298 | RGS3 | 0.9567 | 87.52 | 0.001445 |
| 299 | RIN2 | 1 | 607.6 | 0.00234 |
| 300 | RNF207_B | 0.9643 | 1.07E+09 | 0.9941 |
| 301 | RNF207_C | 1 | 1.68E+09 | 0.9946 |
| 302 | RNF220_B | 0.9733 | 56.55 | 0.008502 |
| 303 | RNF44 | 0.9667 | 8.01E+08 | 0.9936 |
| 304 | RPS6KA1 | 1 | 206.1 | 0.0009877 |
| 305 | RPTOR | 1 | 1.05E+09 | 0.9946 |
| 306 | SELPLG | 1 | 211.9 | 0.001638 |
| 307 | SGK1 | 1 | 76.59 | 0.005985 |
| 308 | SLC2A1 | 1 | 121.9 | 0.0002754 |
| 309 | SMARCA4 | 1 | 474.8 | 0.001605 |
| 310 | SPTBN1 | 0.9667 | 4.93E+08 | 0.9946 |
| 311 | SYN3 | 0.9667 | 1.36E+09 | 0.9948 |
| 312 | SYTL1 | 0.9533 | 223.1 | 0.00105 |
| 313 | TFAP2E | 0.9933 | 208.2 | 0.004403 |
| 314 | TFEB | 1 | 9.96E+08 | 0.9938 |
| 315 | THAP4 | 0.9667 | 9.94E+08 | 0.9953 |
| 316 | TOX | 0.9667 | 1.18E+09 | 0.9949 |
| 317 | TRABD | 1 | 1.93E+09 | 0.993 |
| 318 | TRH | 1 | 7.79E+08 | 0.9939 |
| 319 | TXNRD2 | 1 | 2.90E+09 | 0.9951 |
| 320 | UST | 1 | 147.7 | 0.002184 |
| 321 | WDR66 | 0.9917 | 175.4 | 0.004255 |

### TELQAS Oligonucleotides

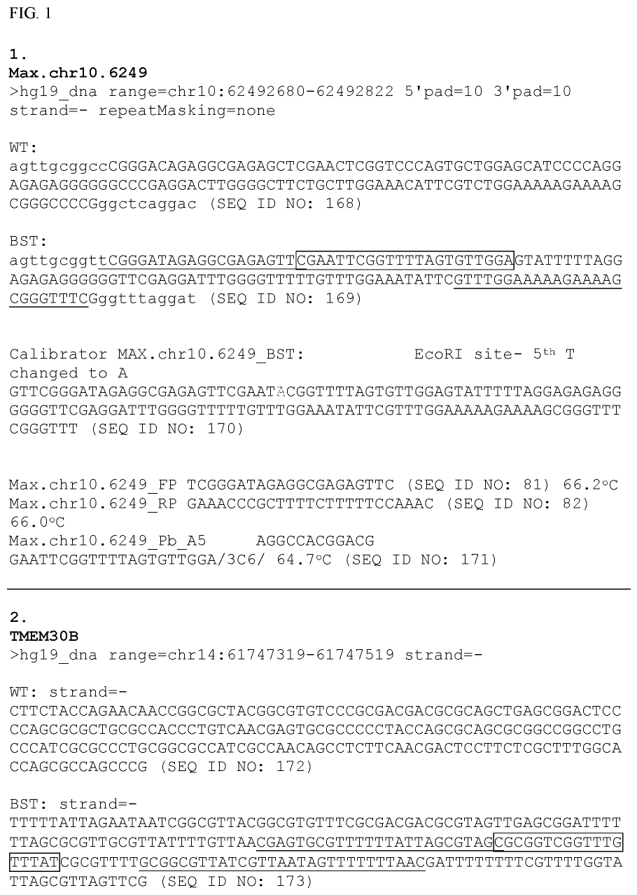

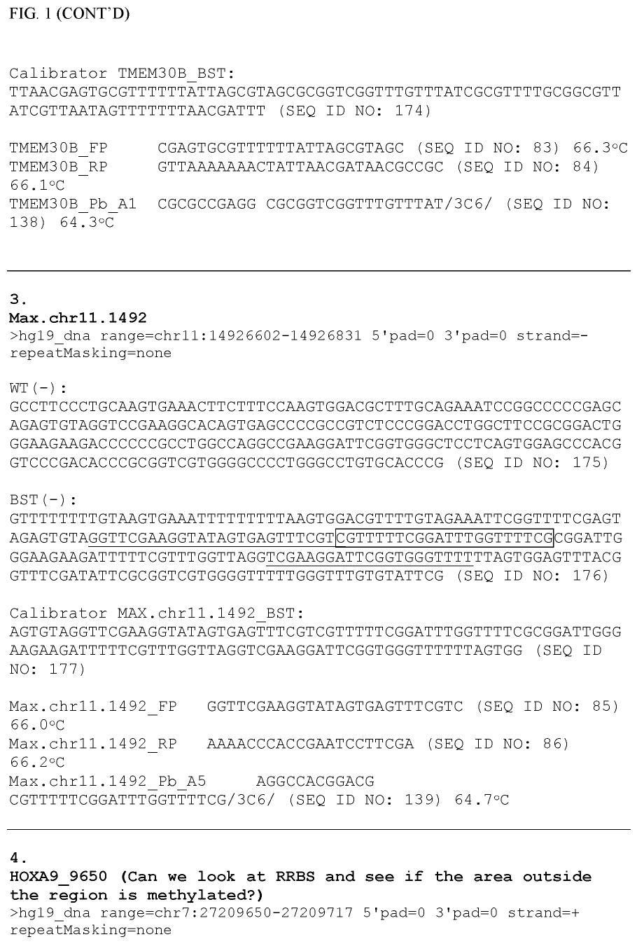

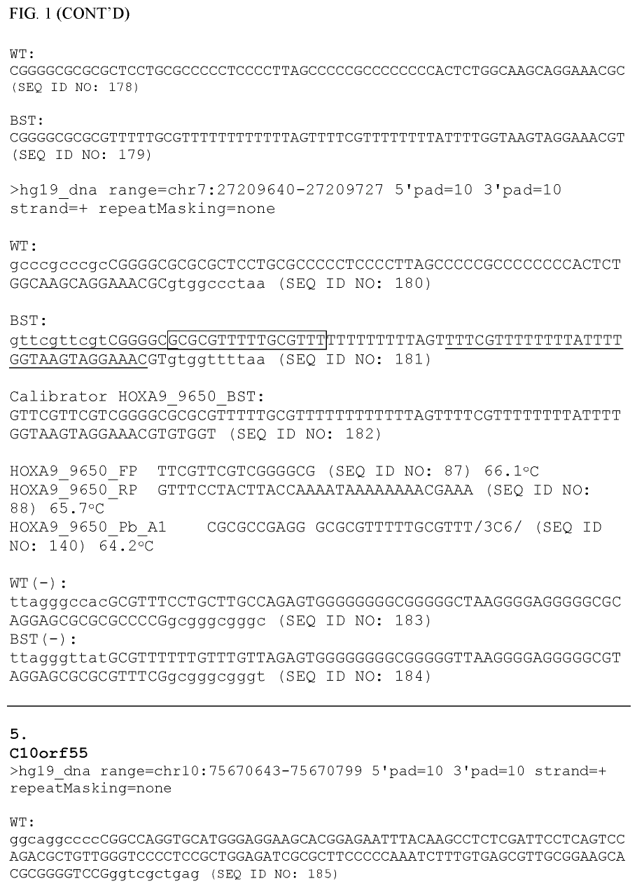

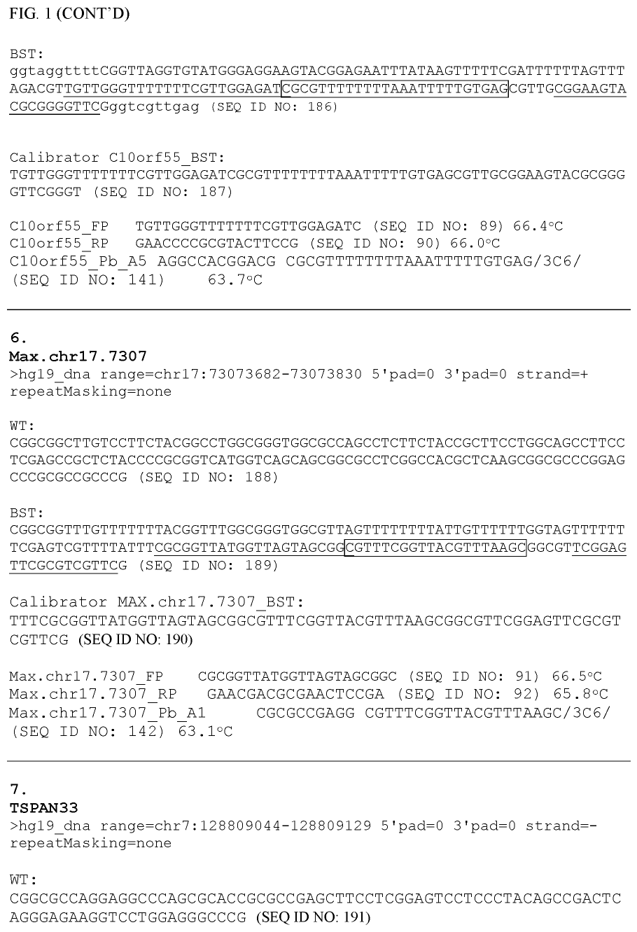

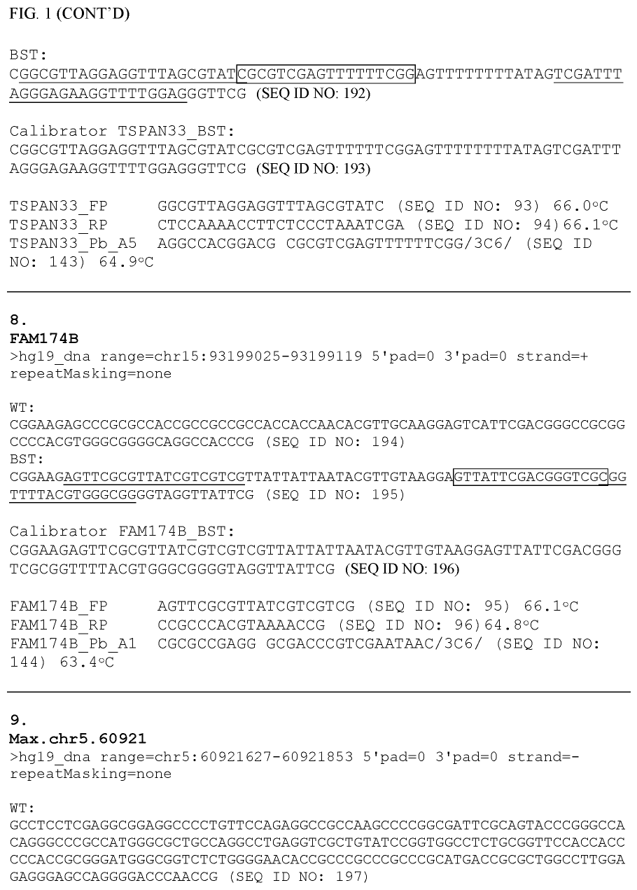

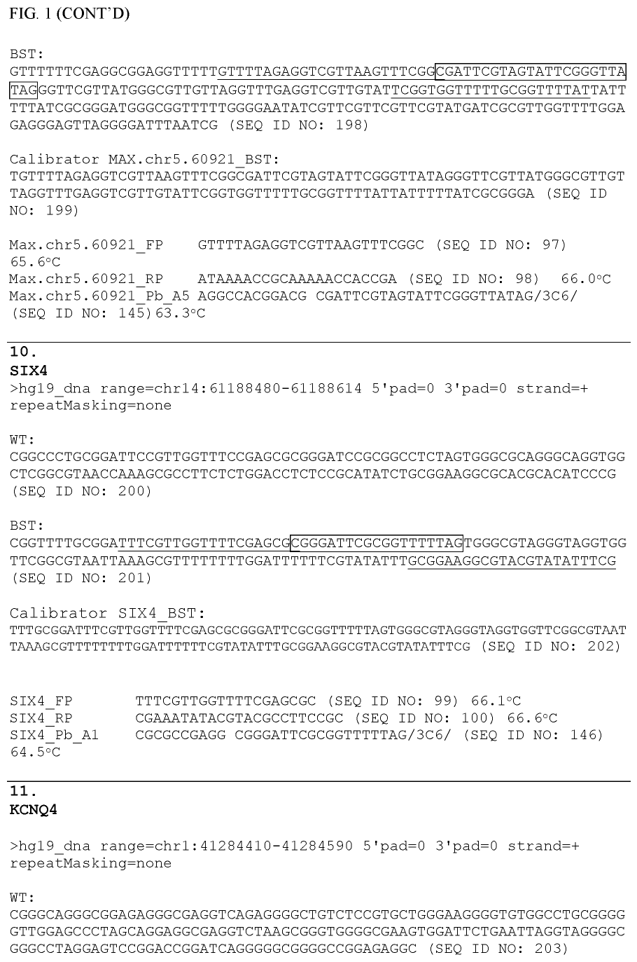

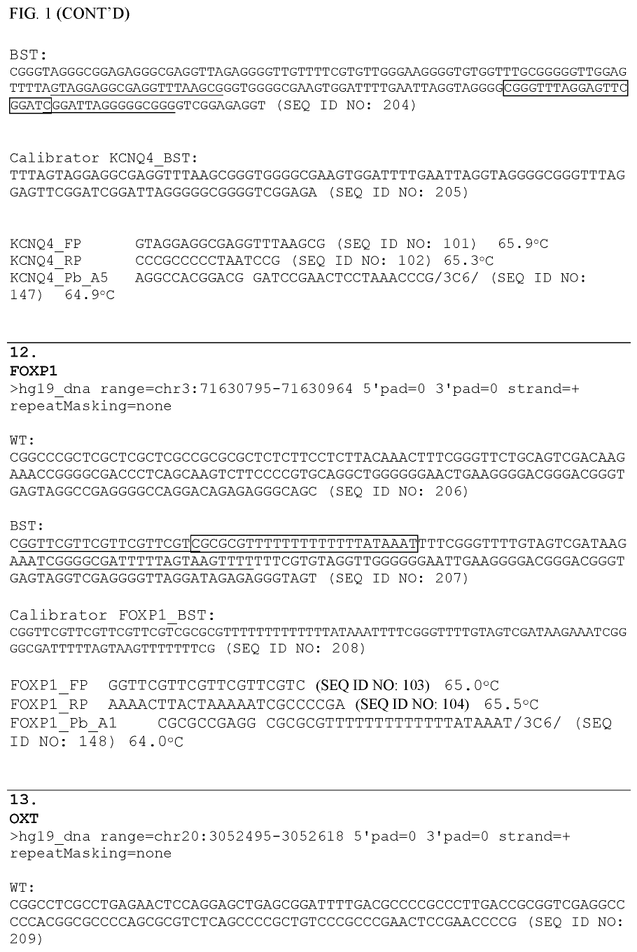

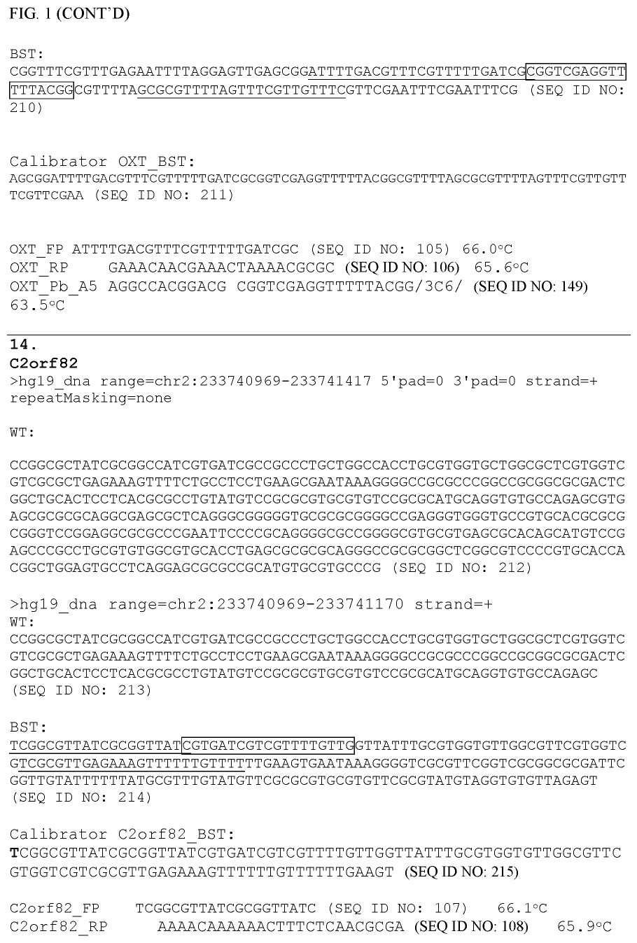

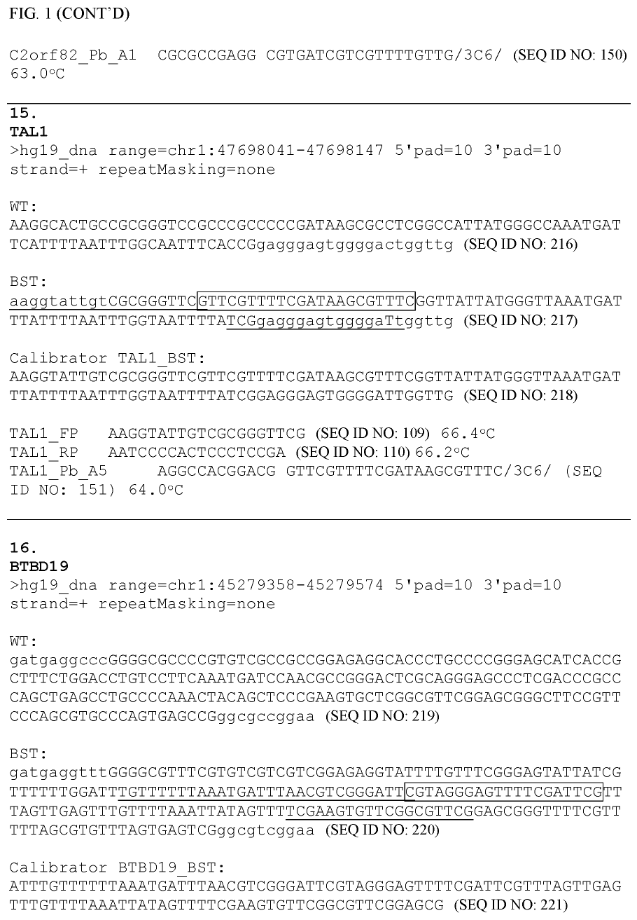

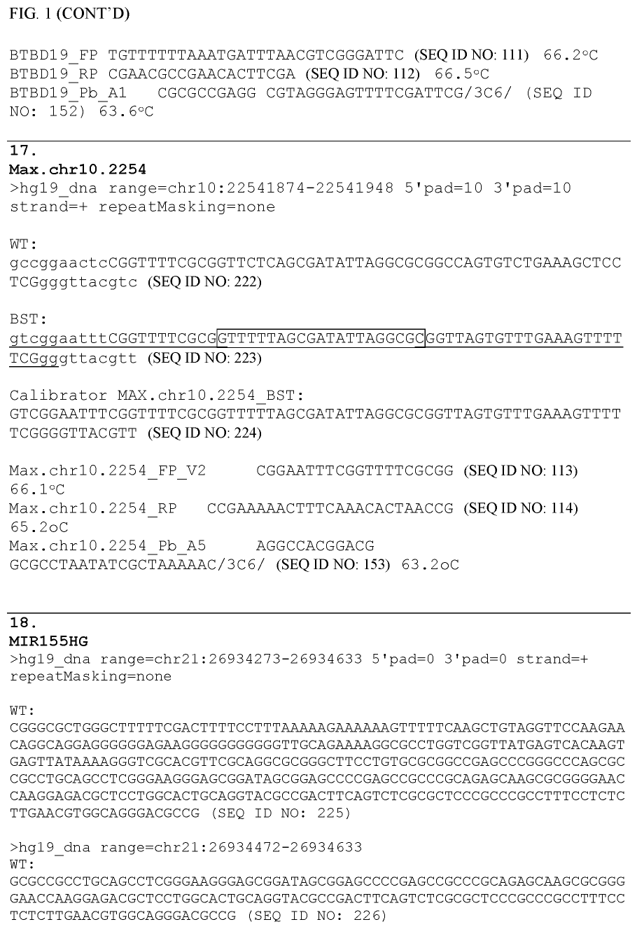

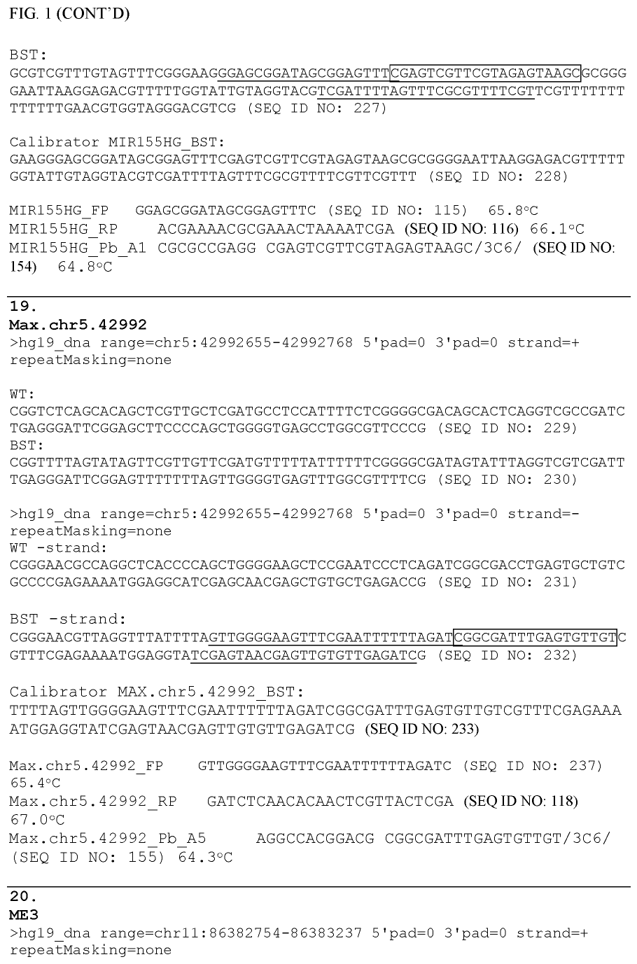

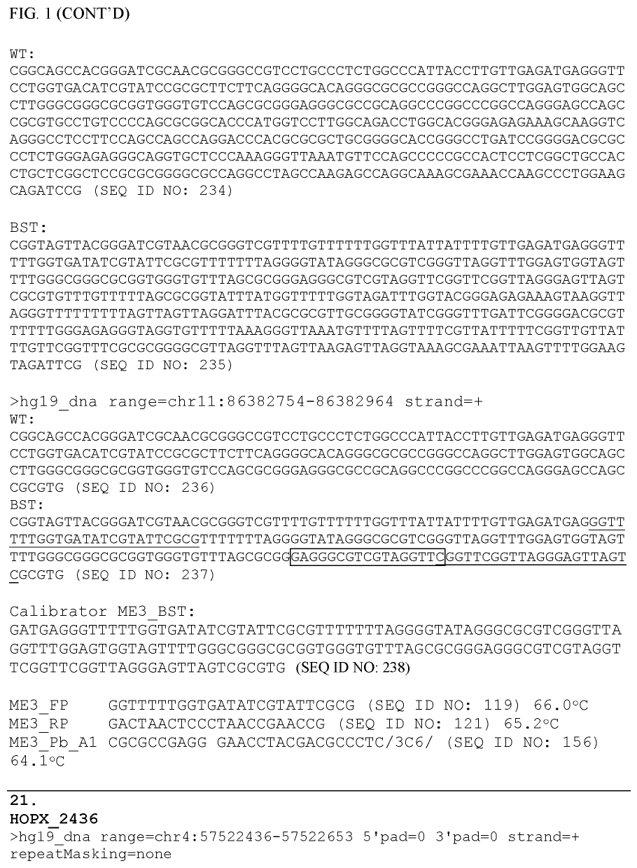

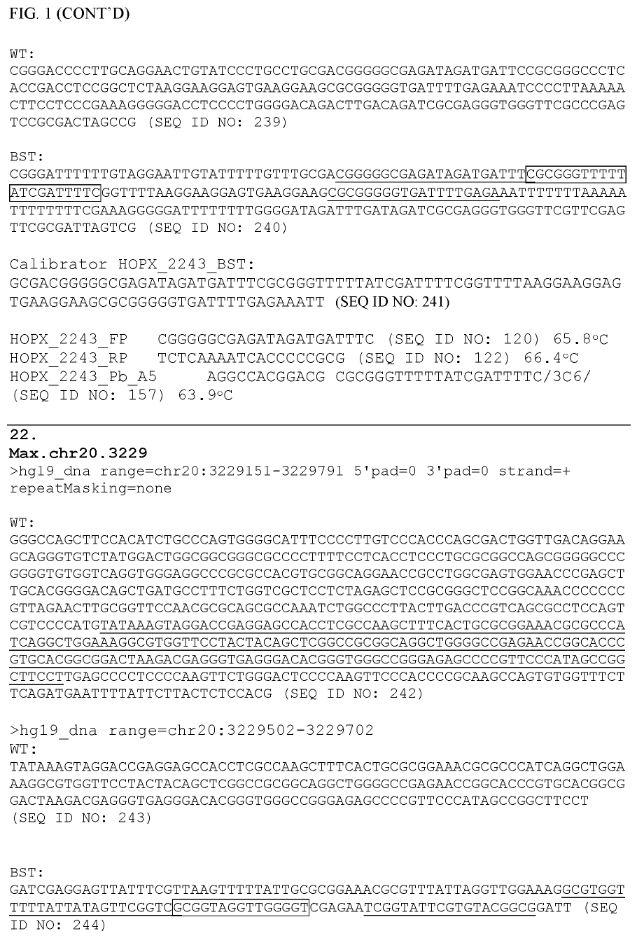

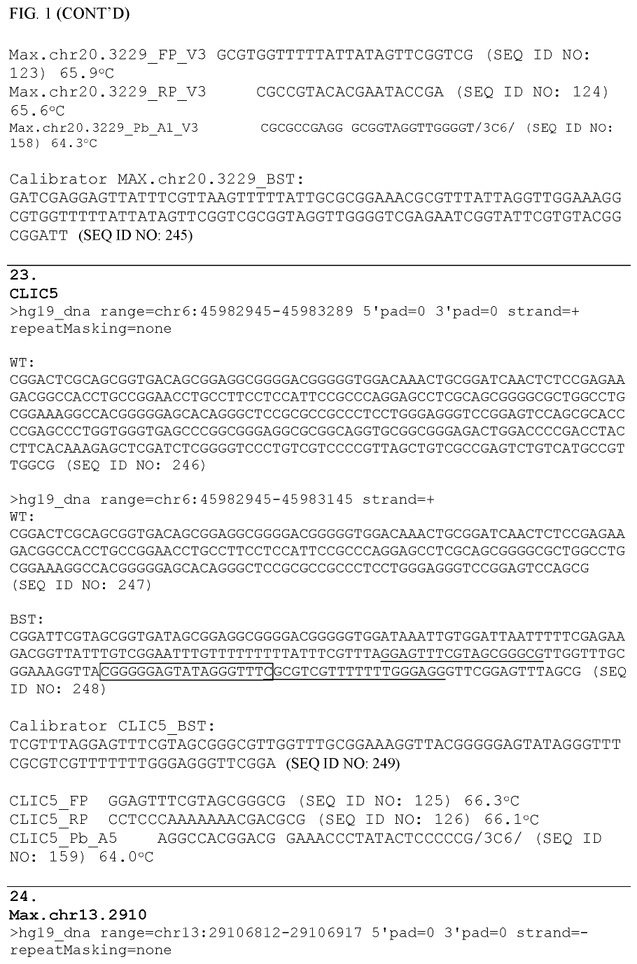

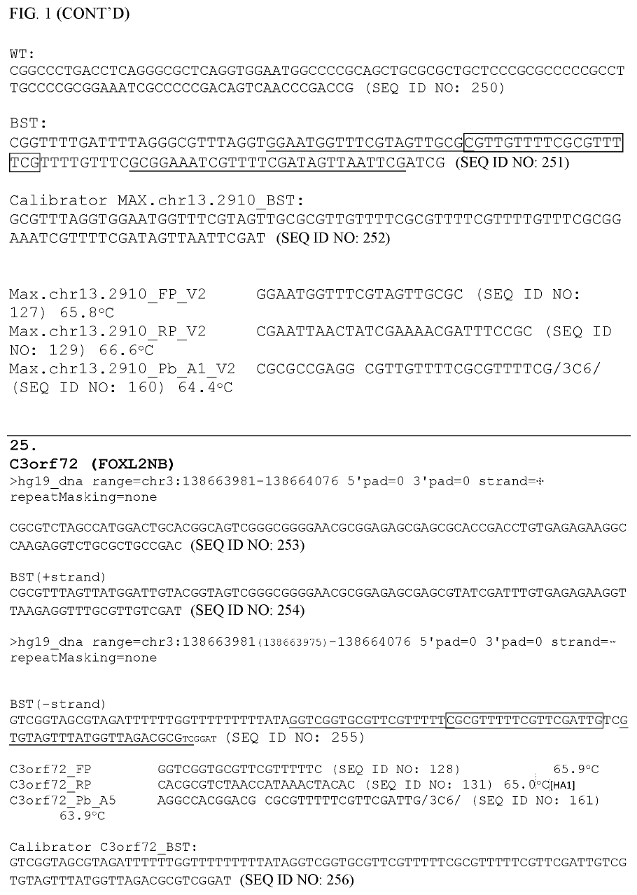

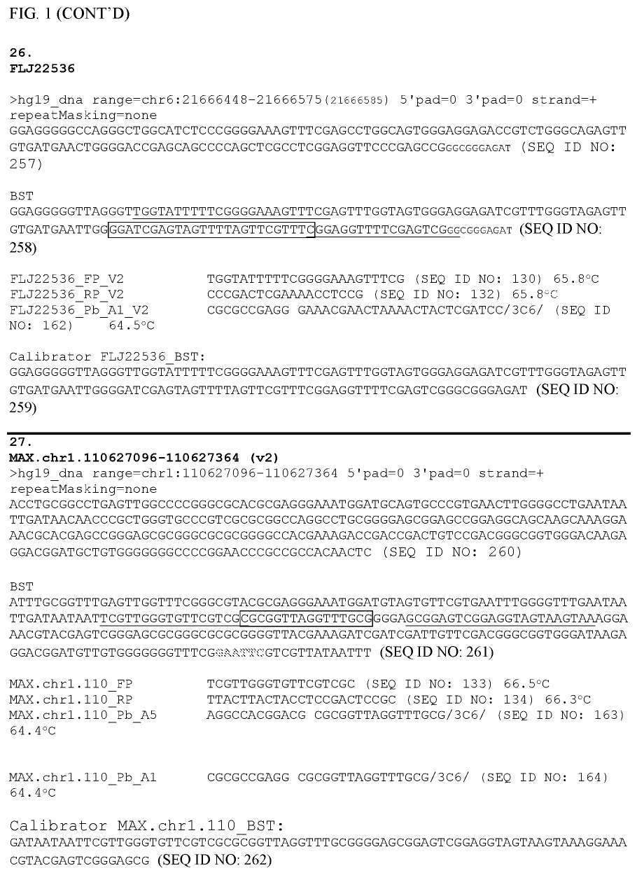

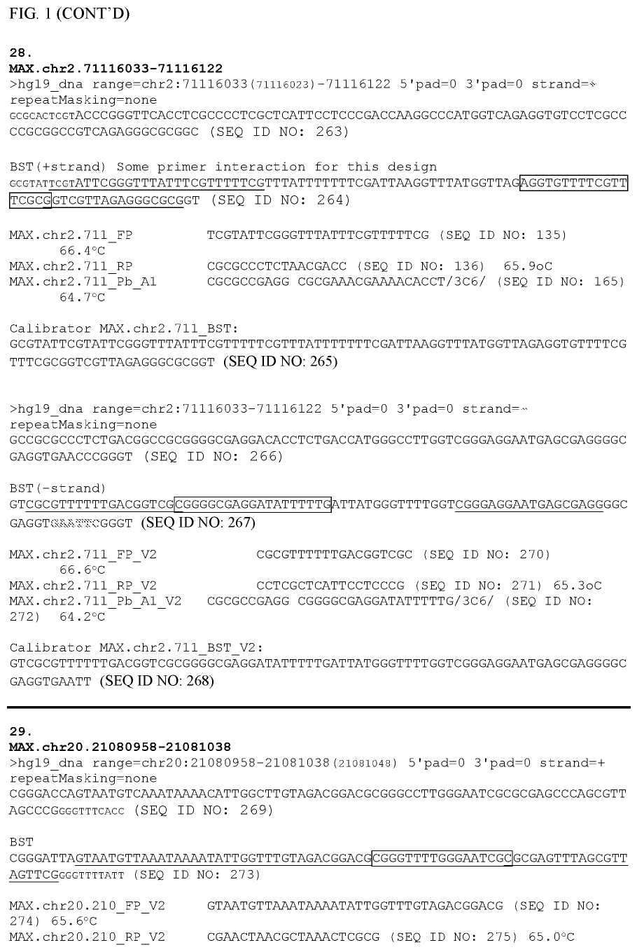

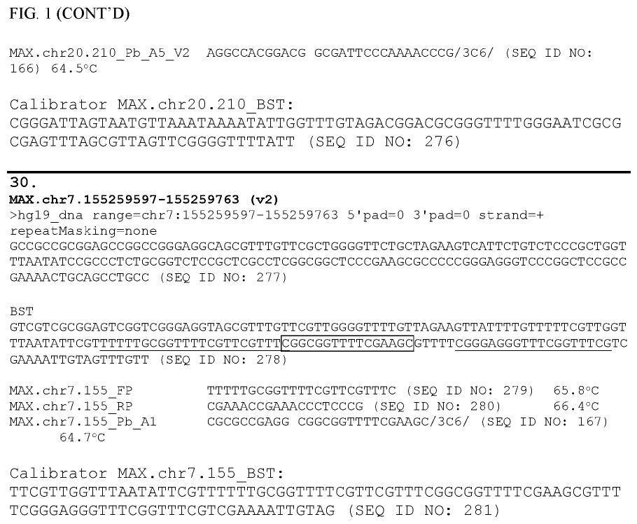

Target enrichment long-probe quantitative amplified signal (TELQAS) (see, Kisiel J B, et al., Hepatology. 2018 Aug. 31) oligos (forward invasive primer, reverse primer, flap probe) were designed to CpG motifs within several of the markers as shown in FIG. 1 and Table 9 for use in, for example, detecting the presence or absence of such markers in tissue and plasma samples. Table 10 refers to primers and Table 11 refers to probes configured for use in a TELQAS assay.

TABLE 9

| | | | DMR Start-End Positions |
|---|---|---|---|
| DMR No. | Gene Annotation | Chromosome No. | (GRCh37/hg19) |
| 322 | MAX.chr10.62492680-62492822 | 10 | 62492680-62492822 |
| 323 | TMEM30B_B | 14 | 61747319-61747519 |
| 324 | MAX.chr11:14926602-14926831 | 11 | 14926602-14926831 |
| 325 | HOXA9_9650 | 7 | 27209650-27209717 |
| 326 | C10orf55 B | 10 | 75670643-75670799 |
| 73 | MAX.chr17.73073682-73073830 | 17 | 73073682-73073830 |
| 122 | TSPAN33 | 7 | 128809044-128809129 |
| 327 | FAM174B_C | 15 | 93199025-93199119 |
| 86 | MAX.chr5.60921627-60921853 | 5 | 60921627-60921853 |
| 111 | SIX4_A | 14 | 61188480-61188614 |
| 49 | KCNQ4_A | 1 | 41284410-41284590 |
| 32 | FOXP1 | 3 | 71630795-71630964 |
| 105 | OXT | 20 | 3052495-3052618 |
| 328 | C2orf82_B | 2 | 233740969-233741417 |
| 329 | TAL1_B | 1 | 47698041-47698147 |
| 330 | BTBD19_B | 1 | 45279358-45279574 |
| 331 | MAX.chr10.22541874-22541948 | 10 | 22541874-22541948 |
| 99 | MIR155HG | 21 | 26934273-26934633 |
| 85 | MAX.chr5.42992655-42992768 | 5 | 42992655-42992768 |
| 97 | ME3 | 11 | 86382754-86383237 |
| 45 | HOPX | 4 | 57522436-57522653 |
| 81 | MAX.chr20.3229151-3229791 | 20 | 3229151-3229791 |
| 21 | CLIC5 | 6 | 45982945-45983289 |
| 66 | MAX.chr13.29106812-29106917 | 13 | 29106812-29106917 |
| 16 | FOXL2NB | 3 | 138663981-138664076 |
| 136 | FLJ22536_A | 6 | 21666448-21666575 |
| 146 | MAX.chr1.110627096-110627364 | 1 | 110627096-110627364 |
| 151 | MAX.chr2.71116033-71116122 | 2 | 71116033-71116122 |
| 152 | MAX.chr20.21080958-21081038 | 20 | 21080958-21081038 |
| 153 | MAX.chr7.155259597-155259763 | 7 | 1552596597-155259763 |

TABLE 10

| | | Seq | | Seq | |
|---|---|---|---|---|---|
| DMR | | ID | Forward Primer 5′-3′ | ID | Reverse Primer 5′-3′ |
| No. | Gene Annotation | No. | Sequence | No. | Sequence |
| 322 | MAX.chr10.62492 | 81 | TCGGGATAGAGGCGAG | 82 | GAAACCCGCTTTTCTT |
| | 680-62492822 | | AGTTC | | TTTCCAAAC |
| 323 | TMEM30B_B | 83 | CGAGTGCGTTTTTTATTA | 84 | GTTAAAAAAACTATTA |
| | | | GCGTAGC | | ACGATAACGCCGC |
| 324 | MAX.chr11:14926 | 85 | GGTTCGAAGGTATAGTG | 86 | AAAACCCACCGAATC |
| | 602-14926831 | | AGTTTCGTC | | CTTCGA |
| 325 | HOXA9_9650 | 87 | TTCGTTCGTCGGGGCG | 88 | GTTTCCTACTTACCAA |
| | | | | | AATAAAAAAAACGAAA |
| 326 | C10orf55_B | 89 | TGTTGGGTTTTTTTCGTT | 90 | GAACCCCGCGTACTT |
| | | | GGAGATC | | CCG |
| 73 | MAX.chr17.73073 | 35 | TTTTTCGAGTCGTTTTAT | 36 | GAACTCCGAACGCCG |
| | 682-73073830 | | TTCGCGG | | CTTAAACGTA |
| 73 | MAX.chr17.73073 | 91 | CGCGGTTATGGTTAGTA | 92 | GAACGACGCGAACTC |
| | 682-73073830 | | GCGGC | | CGA |
| 122 | TSPAN33 | 79 | CGGTTGTAGGGAGGATT | 80 | ACGCCAAAAAACCCA |
| | | | TCGAGGAAGTTC | | ACGCA |
| 122 | TSPAN33 | 93 | GGCGTTAGGAGGTTTAG | 94 | CTCCAAAACCTTCTC |
| | | | CGTATC | | CCTAAATCGA |
| 327 | FAM174B_C | 95 | AGTTCGCGTTATCGTCG | 96 | CCGCCCACGTAAAAC |
| | | | TCG | | CG |
| 86 | MAX.chr5.609216 | 43 | TCGTAGAGGTTATCGGA | 44 | AATACCCGAACCACA |
| | 27-60921853 | | TATAGCGA | | AAACCCGCC |
| 86 | MAX.chr5.609216 | 97 | GTTTTAGAGGTCGTTAA | 98 | ATAAAACCGCAAAAA |
| | 27-60921853 | | GTTTCGGC | | CCACCGA |
| 111 | SIX4_A | 67 | GGTGGTTCGGCGTAATT | 68 | CGTACGCCTTCCGCA |
| | | | AAAGCGT | | AATATACGAA |
| 111 | SIX4_A | 99 | TTTCGTTGGTTTTCGAG | 100 | CGAAATATACGTACG |
| | | | CGC | | CCTTCCGC |
| 49 | KCNQ4_A | 23 | GGGCGAGGTTAGAGGG | 24 | GCCTCCTACTAAAAC |
| | | | GTTGTTHC | | TCCAACCCCCG |
| 49 | KCNQ4_A | 101 | GTAGGAGGCGAGGTTTA | 102 | CCCGCCCCCTAATCC |
| | | | AGCG | | G |
| 32 | FOXP1 | 13 | GGGGCGATTTTTAGTAA | 14 | CTACCCTCTCTATCCT |
| | | | GTTTTTTTCGT | | AACCCCTCGAC |
| 32 | FOXP1 | 103 | GGTTCGTTCGTTCGTTC | 104 | AAAACTTACTAAAAAT |
| | | | GTC | | CGCCCCGA |
| 105 | OXT | 61 | CGTTTGAGAATTTTAGG | 62 | CGAAACAACGAAACT |
| | | | AGTTGAGCGG | | AAAACGCGCT |
| 105 | OXT | 105 | ATTTTGACGTTTCGTTTT | 106 | GAAACAACGAAACTA |
| | | | TGATCGC | | AAACGCGC |
| 328 | C2orf82_B | 107 | TCGGCGTTATCGCGGTT | 108 | AAAACAAAAAACTTTC |
| | | | ATC | | TCAACGCGA |
| 329 | TAL1_B | 109 | AAGGTATTGTCGCGGGT | 110 | AATCCCCACTCCCTC |
| | | | TCG | | CGA |
| 330 | BTBD19_B | 111 | TGTTTTTTAAATGATTTA | 112 | CGAACGCCGAACACT |
| | | | ACGTCGGGATTC | | TCGA |
| 331 | MAX.chr10.22541 | 113 | CGGAATTTCGGTTTTCG | 114 | CCGAAAAACTTTCAAA |
| | 874-22541948 | | CGG | | CACTAACCG |
| 99 | MIR155HG | 53 | CGGATAGCGGAGTTTCG | 54 | AAACGTCTCCTTAATT |
| | | | AGTCGTTC | | CCCCGCGCTT |
| 99 | MIR155HG | 55 | TAAGCGCGGGGAATTAA | 56 | CGAAAACGCGAAACT |
| | | | GGAGACGT | | AAAATCGACGTA |
| 99 | MIR155HG | 115 | GGAGCGGATAGCGGAG | 116 | ACGAAAACGCGAAAC |
| | | | TTTC | | TAAAATCGA |
| 85 | MAX.chr5.429926 | 41 | GTTGGGGAAGTTTCGAA | 42 | TCACCCCAACTAAAA |
| | 55-42992768 | | TTTTTTAGATC | | AAACTCCGAA |
| 85 | MAX.chr5.429926 | 116 | GGGGCGATAGTATTTAG | 117 | GATCTCAACACAACT |
| | 55-42992768 | | GTCGTCGA | | CGTTACTCGA |
| 97 | ME3 | 51 | GTTGCGGGGTATCGGGT | 52 | CCTACCCTCTCCCAA |
| | | | TTGATTC | | AAAACGCGTC |
| 97 | ME3 | 118 | GGTTTTTGGTGATATCG | 119 | GACTAACTCCCTAAC |
| | | | TATTCGCG | | CGAACCG |
| 45 | HOPX | 17 | TTGTATTTTTGTTTGCGA | 18 | CCGAAAATCGATAAA |
| | | | CGGGGGC | | AACCCGCGAA |
| 45 | HOPX | 119 | CGGGGGCGAGATAGAT | 120 | TCTCAAAATCACCCC |
| | | | GATTTC | | CGCG |
| 81 | MAX.chr20.32291 | 121 | GCGTGGTTTTTATATAG | 122 | CGCCGTACACGAATA |
| | 51-3229791 | | TTCGGTCG | | CCGA |
| 21 | CLIC5 | 7 | GGCGTTGGTTTGCGGAA | 8 | CACCCACCAAAACTC |
| | | | AGGTTAC | | GAAATACGCT |
| 21 | CLIC5 | 123 | GGAGTTTCGTAGCGGGC | 124 | CCTCCCAAAAAAACG |
| | | | G | | ACGCG |
| 66 | MAX.chr13.29106 | 33 | CGGTTTTGATTTTAGGG | 34 | TTAACTATCGAAAACG |
| | 812-29106917 | | CGT | | ATTTCCGCGAA |
| 66 | MAX.chr13.29106 | 125 | GGAATGGTTTCGTAGTT | 126 | CGAATTAACTATCGAA |
| | 812-29106917 | | GCGC | | AACGATTTCCGC |
| 16 | FOXL2NB | 5 | TATGGATTGTACGGTAG | 6 | ACGCAAACCTCTTAA |
| | | | TCGGGCGG | | CCTTCTCTCACAAATC |
| | | | | | G |
| 16 | FOXL2NB | 126 | GGTCGGTGCGTTCGTTT | 127 | CACGCGTCTAACCAT |
| | | | TTC | | AAACTACAC |
| 136 | FLJ22536_A | 11 | GAGTTTGGTAGTGGGAG | 12 | CCGAAACGAACTAAA |
| | | | GAGATCGT | | ACTACTCGAT |
| 136 | FLJ22536_A | 127 | TGGTATTTTTCGGGGAA | 128 | CCCGACTCGAAAACC |
| | | | AGTTTCG | | TCCG |
| 146 | MAX.chr1.110627 | 27 | CGAAAGATCGATCGATT | 28 | TTATAACGACGAATTC |
| | 096-110627364 | | GTTCGACGG | | CGAAA |
| 146 | MAX.chr1.110627 | 129 | TCGTTGGGTGTTCGTCG | 130 | TTACTTACTACCTCCG |
| | 096-110627364 | | C | | ACTCCGC |
| 151 | MAX.chr2.711160 | 37 | ATTTTTGATTATGGGTTT | 38 | GAATTCACCTCGCCC |
| | 33-71116122 | | TGGTCGG | | CTCGCT |
| 151 | MAX.chr2.711160 | 131 | TCGTATTCGGGTTTATTT | 132 | CGCGCCCTCTAACGA |
| | 33-71116122 | | CGTTTTTCG | | CC |
| 152 | MAX.chr20.21080 | 39 | TAAATAAAATATTGGTTT | 40 | CGAACTAACGCTAAA |
| | 958-21081038 | | GTAGACGGACGCGG | | CTCGCGCGAT |
| 152 | MAX.chr20.21080 | 133 | GTAATGTTAAATAAAATA | 134 | CGAACTAACGCTAAA |
| | 958-21081038 | | TTGGTTTGTAGACGGAC | | CTCGCG |
| | | | G | | |
| 153 | MAX.chr7.155259 | 49 | TTTTCGGCGGAGTCGGG | 50 | GATCTCCGCTCGCCT |
| | 597-155259763 | | ATTTTTTC | | CGACG |
| | (v2) | | | | |
| 153 | MAX.chr7.155259 | 135 | TTTTTGCGGTTTTCGTTC | 136 | CGAAACCGAAACCCT |
| | 597-155259763 | | GTTTC | | CCCG |
| | (v2) | | | | |

TABLE 11

| DMR | | | |
|---|---|---|---|
| No. | Gene Annotation | SEQ ID NO | Probe Sequence |
| 322 | MAX.chr10.62492680-62492822 | 137 | AGGCCACGGACG |
| | | | CGAATTCGGTllTAGTGTTGGA/3C6/ |
| 323 | TMEM30B_B | 138 | CGCGCCGAGG |
| | | | CGCGGTCGGTTTGTTTAT/3C6/ |
| 324 | MAX.chr11:14926602-14926831 | 139 | AGGCCACGGACG |
| | | | CGTTTTTCGGATTTGGTTTTCG/3C6/ |
| 325 | HOXA9_9650 | 140 | CGCGCCGAGG |
| | | | GCGCGTTTTTGCGTTT/3C6/ |
| 326 | C10orf55_B | 141 | AGGCCACGGACG |
| | | | CGCGTTTTTTTTAAATTTTTGTGAG/3C6/ |
| 73 | MAX.chr17.73073682-73073830 | 142 | CGCGCCGAGG |
| | | | CGTTTCGGTTACGTTTAAGC/3C6/ |
| 122 | TSPAN33 | 143 | AGGCCACGGACG |
| | | | CGCGTCGAGTTTTTTCGG/3C6/ |
| 327 | FAM174B_C | 144 | CGCGCCGAGG |
| | | | GCGACCCGTCGAATAAC/3C6/ |
| 86 | MAX.ch_r5.60921627-60921853 | 145 | AGGCCACGGACG |
| | | | CGATTCGTAGTATTCGGGTTATAG/3C6/ |
| 111 | SIX4_A | 146 | CGCGCCGAGG |
| | | | CGGGATTCGCGGTTTTTAG/3C6/ |
| 49 | KCNQ4_A | 147 | AGGCCACGGACG |
| | | | GATCCGAACTCCTAAACCCG/3C6/ |
| 32 | FOXP1 | 148 | CGCGCCGAGG |
| | | | CGCGCGTTTTTTTTTTTTTATAAAT/3C6/ |
| 105 | OXT | 149 | AGGCCACGGACG |
| | | | CGGTCGAGGTTTTTACGG/3C6/ |
| 328 | C2orf82_B | 150 | CGCGCCGAGG |
| | | | CGTGATCGTCGTTTTGTTG/3C6/ |
| 329 | TAL1_B | 151 | AGGCCACGGACG |
| | | | GTTCGTTTTCGATAAGCGTTTC/3C6/ |
| 330 | BTBD19_B | 152 | CGCGCCGAGG |
| | | | CGTAGGGAGTTTTCGATTCG/3C6/ |
| 331 | MAX.chr10.22541874-22541948 | 153 | AGGCCACGGACG |
| | | | GCGCCTAATATCGCTAAAAAC/3C6/ |
| 99 | MIR155HG | 154 | CGCGCCGAGG |
| | | | CGAGTCGTTCGTAGAGTAAGC/3C6/ |
| 85 | MAX.chr5.42992655-42992768 | 155 | AGGCCACGGACG |
| | | | CGGCGATTTGAGTGTTGT/3C6/ |
| 97 | ME3 | 156 | CGCGCCGAGG |
| | | | GAACCTACGACGCCCTC/3C6/ |
| 45 | HOPX | 157 | AGGCCACGGACG |
| | | | CGCGGGTTTTTATCGATTTTC/3C6/ |
| 81 | MAX.chr20.3229151-3229791 | 158 | CGCGCCGAGG |
| | | | GCGGTAGGTTGGGGT/3C6/ |
| 21 | CLIC5 | 159 | AGGCCACGGACG |
| | | | GAAACCCTATACTCCCCCG/3C6/ |
| 66 | MAX.chr13.29106812-29106917 | 160 | CGCGCCGAGG |
| | | | CGTTGTTTTCGCGTTTTCG/3C6/ |
| 16 | FOXL2NB | 161 | AGGCCACGGACG |
| | | | CGCGTTTTTCGTTCGATTG/3C6/ |
| 136 | FLJ22536_A | 162 | CGCGCCGAGG |
| | | | GAAACGAACTAAAACTACTCGATCC/3C6/ |
| 146 | MAX.chr1.110627096- | 163 | AGGCCACGGACG |
| | 110627364 | | CGCGGTTAGGTTTGCG/3C6/ |
| 146 | MAX.chr1.110627096- | 164 | CGCGCCGAGG |
| | 110627364 | | CGCGGTTAGGTTTGCG/3C6/ |
| 151 | MAX.chr2.71116033-71116122 | 165 | CGCGCCGAGG |
| | | | CGCGAAACGAAAACACCT/3C6/ |
| 152 | MAX.chr20.21080958-21081038 | 166 | AGGCCACGGACG |
| | | | GCGATTCCCAAAACCCG/3C6/ |
| 153 | MAX.chr7.155259597- | 167 | CGCGCCGAGG |
| | 155259763 | | CGGCGGTTTTCGAAGC/3C6/ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method, comprising:
measuring a methylation level for one or more genes in a biological sample of a human individual through
treating genomic DNA in the biological sample with a reagent that modifies DNA in a methylation-specific manner;
amplifying the treated genomic DNA using a set of primers for the selected one or more genes; and
determining the methylation level of the one or more genes by polymerase chain reaction, nucleic acid sequencing, mass spectrometry, methylation-specific nuclease, mass-based separation, and target capture;
wherein the one or more genes is selected from
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33; or
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73; or
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763; or
MAX.chr20.21080958-21081038, MAX.chr7.155259597-155259763, FOXL2NB, and HOXA9_A; or
One or more markers recited in Table 8A.

2. The method of claim 1, wherein the DNA is treated with a reagent that modifies DNA in a methylation-specific manner.

3. The method of claim 2, wherein the reagent comprises one or more of a methylation-sensitive restriction enzyme, a methylation-dependent restriction enzyme, and a bisulfite reagent.

4. The method of claim 3, wherein the DNA is treated with a bisulfite reagent to produce bisulfite-treated DNA.

5. The method of claim 1, wherein the measuring comprises multiplex amplification.

6. The method of claim 1, wherein measuring the amount of at least one methylated marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

7. The method of claim 1, wherein the sample comprises one or more of a plasma sample, a blood sample, a fine needle aspirate sample, or a tissue sample (e.g., skin tissue).

8. The method of claim 1, wherein the set of primers for the selected one or more genes is recited in Table 3 or 10.

9. The method of claim 1, wherein the method is used for detecting the presence or absence of melanoma in the biological sample from the human.

10. A method of characterizing a sample, comprising:
a) measuring an amount of at least one methylated marker gene in DNA from the sample, wherein the at least one methylated marker gene is one or more genes selected from
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33; or
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763, or
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73; or
MAX.chr20.21080958-21081038, MAX.chr7.155259597-155259763, FOXL2NB, and HOXA9_A; or
One or more markers recited in Table 8A;
b) measuring the amount of at least one reference marker in the DNA; and
c) calculating a value for the amount of the at least one methylated marker gene measured in the DNA as a percentage of the amount of the reference marker gene measured in the DNA, wherein the value indicates the amount of the at least one methylated marker DNA measured in the sample.

11. The method of claim 10, wherein the at least one reference marker comprises one or more reference marker selected from B3GALT6 DNA, ZDHHC1 DNA, β-actin DNA, and non-cancerous DNA.

12. The method of claim 10, wherein the sample comprises one or more of a plasma sample, a blood sample, a fine needle aspirate sample, or a tissue sample (e.g., skin tissue).

13. The method of claim 10, wherein the DNA is extracted from the sample.

14. The method of claim 10, wherein the DNA is treated with a reagent that modifies DNA in a methylation-specific manner.

15. The method of claim 14, wherein the reagent comprises one or more of a methylation-sensitive restriction enzyme, a methylation-dependent restriction enzyme, and a bisulfite reagent.

16. The method of claim 15, wherein the DNA is treated with a bisulfite reagent to produce bisulfite-treated DNA.

17. The method of claim 15, wherein the modified DNA is amplified using a set of primers for the selected one or more genes.

18. The method of claim 17, wherein the set of primers for the selected one or more genes is recited in Table 3 or 10.

19. The method of claim 10, wherein measuring amounts of a methylated marker gene comprises using one or more of polymerase chain reaction, nucleic acid sequencing, mass spectrometry, methylation-specific nuclease, mass-based separation, and target capture.

20. The method of claim 19, wherein the measuring comprises multiplex amplification.

21. The method of claim 19, wherein measuring the amount of at least one methylated marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

22. The method of claim 10, wherein the method is used for detecting the presence or absence of melanoma in the biological sample from the human.

23. A method of characterizing a sample, comprising:
a) measuring an amount of at least one methylated marker gene in DNA from the sample, wherein the at least one methylated marker gene is one or more genes selected from:
MAX.chr20.21080958-21081038, MAX.chr7.155259597-155259763, FOXL2NB, and HOXA9_A, or
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763;
b) measuring the amount of at least one reference marker in the DNA; and
c) calculating a value for the amount of the at least one methylated marker gene measured in the DNA as a percentage of the amount of the reference marker gene measured in the DNA, wherein the value indicates the amount of the at least one methylated marker DNA measured in the sample.

24. The method of claim 23, wherein the at least one reference marker comprises one or more reference marker selected from B3GALT6 DNA, ZDHHC1 DNA, R-actin DNA, and non-cancerous DNA.

25. The method of claim 23, wherein the sample comprises one or more of a plasma sample, a blood sample, a fine needle aspirate sample, or a tissue sample (e.g., skin tissue).

26. The method of claim 23, wherein the DNA is extracted from the sample.

27. The method of claim 23, wherein the DNA is treated with a reagent that modifies DNA in a methylation-specific manner.

28. The method of claim 27, wherein the reagent comprises one or more of a methylation-sensitive restriction enzyme, a methylation-dependent restriction enzyme, and a bisulfite reagent.

29. The method of claim 28, wherein the DNA is treated with a bisulfite reagent to produce bisulfite-treated DNA.

30. The method of claim 27, wherein the modified DNA is amplified using a set of primers for the selected one or more genes.

31. The method of claim 30, wherein the set of primers for the selected one or more genes is recited in Table 3 or 10.

32. The method of claim 10, wherein measuring amounts of a methylated marker gene comprises using one or more of polymerase chain reaction, nucleic acid sequencing, mass spectrometry, methylation-specific nuclease, mass-based separation, and target capture.

33. The method of claim 32, wherein the measuring comprises multiplex amplification.

34. The method of claim 32, wherein measuring the amount of at least one methylated marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

35. The method of claim 23, wherein the method is used for detecting the presence or absence of metastatic melanoma in the biological sample from the human.

36. A method for characterizing a biological sample comprising:
(a) measuring a methylation level of a CpG site for one or more genes selected from
c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33; or
MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763; or
AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73; or
MAX.chr20.21080958-21081038, MAX.chr7.155259597-155259763, FOXL2NB, and HOXA9_A; or
One or more markers recited in Table 8A
in a biological sample of a human individual through treating genomic DNA in the biological sample with bisulfite;
amplifying the bisulfite-treated genomic DNA using a set of primers for the selected one or more genes; and
determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
(b) comparing the methylation level to a methylation level of a corresponding set of genes in control samples without melanoma; and
(c) determining that the individual has melanoma when the methylation level measured in the one or more genes is higher than the methylation level measured in the respective control samples.

37. The method of claim 36 wherein the set of primers for the selected one or more genes is recited in Table 3 or 10.

38. The method of claim 36, wherein the biological sample is a plasma sample, a blood sample, a fine needle aspirate sample, or a tissue sample (e.g., skin tissue).

39. The method of claim 36, wherein the one or more genes is described by the genomic coordinates shown in Table 1A, 2A, 7A, 8A, and/or 9.

40. The method of claim 36, wherein said CpG site is present in a coding region or a regulatory region.

41. The method of claim 36, wherein said measuring the methylation level a CpG site for one or more genes comprises a determination selected from the group consisting of determining the methylation score of said CpG site and determining the methylation frequency of said CpG site.

42. A method for characterizing a biological sample comprising:
(a) measuring a methylation level of a CpG site for one or more genes selected from MAX.chr20.21080958-21081038, MAX.chr7.155259597-155259763, FOXL2NB, and HOXA9_A
in a biological sample of a human individual through
treating genomic DNA in the biological sample with bisulfite;
amplifying the bisulfite-treated genomic DNA using a set of primers for the selected one or more genes; and
determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
(b) comparing the methylation level to a methylation level of a corresponding set of genes in control samples without clear cell melanoma; and
(c) determining that the individual has metastatic melanoma when the methylation level measured in the one or more genes is higher than the methylation level measured in the respective control samples.

43. The method of claim 42 wherein the set of primers for the selected one or more genes is recited in Table 3.

44. The method of claim 42, wherein the biological sample is a plasma sample, a blood sample, a fine needle aspirate sample, or a tissue sample (e.g., skin tissue).

45. The method of claim 42, wherein the one or more genes is described by the genomic coordinates shown in Table TA and/or 2A.

46. The method of claim 42, wherein said CpG site is present in a coding region or a regulatory region.

47. The method of claim 42, wherein said measuring the methylation level a CpG site for one or more genes comprises a determination selected from the group consisting of determining the methylation score of said CpG site and determining the methylation frequency of said CpG site.

48. A system for characterizing a sample obtained from a human subject, the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a melanoma-associated methylation state.

49. The system of claim 48 wherein the sample comprises a nucleic acid comprising a DMR.

50. The system of claim 48 further comprising a component for isolating a nucleic acid.

51. The system of claim 48 further comprising a component for collecting a sample.

52. The system of claim 48 wherein the sample is a stool sample, a tissue sample, a skin tissue sample, a fine needle aspirate sample, a blood sample, a serum sample, a plasma sample, or a urine sample.

53. The system of claim 48 wherein the database comprises nucleic acid sequences comprising a DMR.

54. The system of claim 48 wherein the database comprises nucleic acid sequences from subjects who do not have melanoma.

55. A kit comprising:
1) a bisulfite reagent; and
2) a control nucleic acid comprising a sequence from a DMR selected from a group consisting of DMR 1-331 from Table 1A, 2A, 7A, 8A, and 9, and having a methylation state associated with a subject who does not have melanoma.

56. A kit comprising a bisulfite reagent and an oligonucleotide according to SEQ ID NOS 1-80.

57. A kit comprising a sample collector for obtaining a sample from a subject; reagents for isolating a nucleic acid from the sample; a bisulfite reagent; and an oligonucleotide according to SEQ ID NOS 1-80.

58. The kit according to claim 57 wherein the sample is a stool sample, a tissue sample, a skin tissue sample, a fine needle aspirate sample, a blood sample, a serum sample, a plasma sample, or a urine sample.

59. A composition comprising a nucleic acid comprising a DMR and a bisulfite reagent.

60. A composition comprising a nucleic acid comprising a DMR and an oligonucleotide according to SEQ ID NOS 1-167.

61. A composition comprising a nucleic acid comprising a DMR and a methylation-sensitive restriction enzyme.

62. A composition comprising a nucleic acid comprising a DMR and a polymerase.

63. A method of screening for a melanoma in a sample obtained from a subject, the method comprising:
1) assaying a methylation state of a marker in a sample obtained from a subject; and
2) identifying the subject as having melanoma when the methylation state of the marker is different than a methylation state of the marker assayed in a subject that does not have a neoplasm,
wherein the marker comprises a base in a differentially methylated region (DMR) as provided in Tables 1A, 2A, 7A, 8A, and 9.

64. The method of claim 63 comprising assaying a plurality of markers.

65. The method of claim 63 comprising assaying 2 to 11 markers.

66. The method of claim 63 comprising assaying 12 to 560 markers.

67. The method of claim 63 wherein assaying the methylation state of the marker in the sample comprises determining the methylation state of one base.

68. The method of claim 63 wherein assaying the methylation state of the marker in the sample comprises determining the extent of methylation at a plurality of bases.

69. The method of claim 63 wherein the methylation state of the marker comprises an increased or decreased methylation of the marker relative to a normal methylation state of the marker.

70. The method of claim 63 wherein the methylation state of the marker comprises a different pattern of methylation of the marker relative to a normal methylation state of the marker.

71. The method of claim 63 comprising assaying a methylation state of a forward strand or assaying a methylation state of a reverse strand.

72. The method of claim 63 wherein the marker is a region of 100 or fewer bases.

73. The method of claim 63 wherein the marker is a region of 500 or fewer bases.

74. The method of claim 63 wherein the marker is a region of 1000 or fewer bases.

75. The method of claim 63 wherein the marker is a region of 5000 or fewer bases.

76. The method of claim 63 wherein the marker is one base.

77. The method of claim 63 wherein the marker is in a high CpG density promoter.

78. The method of claim 63 wherein the sample is a stool sample, a tissue sample, a skin tissue sample, a blood sample, a fine needle aspirate sample, or a urine sample.

79. The method of claim 63 wherein the assaying comprises using methylation specific polymerase chain reaction, nucleic acid sequencing, mass spectrometry, methylation specific nuclease, mass-based separation, or target capture.

80. The method of claim 63 wherein the assaying comprises use of a methylation specific oligonucleotide.

81. A method for characterizing a biological sample comprising:
measuring a methylation level of a CpG site for one or more of MAX.chr20.21080958-21081038, MAX.chr7.155259597-155259763, FOXL2NB, and HOXA9_A in a biological sample of a human individual through
treating genomic DNA in the biological sample with bisulfite;
amplifying the bisulfite-treated genomic DNA using primers specific for a CpG site for MAX.chr20.21080958-21081038, primers specific for a CpG site for MAX.chr7.155259597-155259763, primers specific for a CpG site for FOXL2NB, and primers specific for a CpG site for HOXA9_A,
wherein the primers specific for MAX.chr7.155259597-155259763 are capable of binding an amplicon bound by SEQ ID Nos: 47 and 48 or 49 and 50, wherein the amplicon bound by SEQ ID Nos: 47 and 48 or 49 and 50 is at least a portion of a genetic region comprising chromosome 7 coordinates 155259597-155259763,
wherein the primers specific for MAX.chr20.21080958-21081038 are capable of binding an amplicon bound by SEQ ID Nos: 39 and 40, wherein the amplicon bound by SEQ ID Nos: 39 and 40 is at least a portion of a genetic region comprising chromosome 20 coordinates 21080958-21081038,
wherein the primers specific for FOXL2NB are capable of binding an amplicon bound by SEQ ID Nos: 5 and 6, wherein the amplicon bound by SEQ ID Nos: 5 and 6 is at least a portion of a genetic region comprising chromosome 3 coordinates 138663981-138664076; and
wherein the primers specific for HOXA9_A are capable of binding an amplicon bound by SEQ ID Nos: 19 and 20, wherein the amplicon bound by SEQ ID Nos: 19 and 20 is at least a portion of a genetic region comprising chromosome 7 coordinates 27209628-27209739;
determining the methylation level of the CpG site for the one or more of MAX.chr20.21080958-21081038, MAX.chr7.155259597-155259763, FOXL2NB, and HOXA9_A by methylation-specific PCR, quantitative methylation-specific PCR, methylation-sensitive DNA restriction enzyme analysis, quantitative bisulfite pyrosequencing, or bisulfite genomic sequencing PCR.

82. The method of claim 81, wherein the biological sample is a blood sample, a tissue sample, a fine needle aspirate sample, or a skin tissue sample.

83. The method of claim 81, wherein said CpG site is present in a coding region or a regulatory region.

84. The method of claim 81, wherein said measuring the methylation level of the CpG site for the one or more of MAX.chr20.21080958-21081038, MAX.chr7.155259597-155259763, FOXL2NB, and HOXA9_A comprises a determination selected from the group consisting of determining the methylation score of said CpG site and determining the methylation frequency of said CpG site.

85. A method for characterizing a biological sample comprising:
measuring a methylation level of a CpG site for one or more markers selected from c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33 in a biological sample of a human individual through
treating genomic DNA in the biological sample with bisulfite;
amplifying the bisulfite-treated genomic DNA using primers specific for a CpG site for the one or more markers,
wherein the primers specific for each marker are capable of binding an amplicon bound by the respective primer sequences recited in Table 3, wherein the amplicon bound by the respective primer sequences is at least a portion of a genetic region comprising the respective chromosomal region recited in Table 1A or Table 2A;
determining the methylation level of the CpG site for the one or more markers by methylation-specific PCR, quantitative methylation-specific PCR, methylation-sensitive DNA restriction enzyme analysis, quantitative bisulfite pyrosequencing, or bisulfite genomic sequencing PCR.

86. The method of claim 85, wherein the biological sample is a blood sample, a tissue sample, a fine needle aspirate sample, or a skin tissue sample.

87. The method of claim 85, wherein said CpG site is present in a coding region or a regulatory region.

88. The method of claim 85, wherein said measuring the methylation level of the CpG site for the one or more markers comprises a determination selected from the group consisting of determining the methylation score of said CpG site and determining the methylation frequency of said CpG site.

89. A method for characterizing a biological sample comprising:
measuring a methylation level of a CpG site for one or more markers recited in Table 8A in a biological sample of a human individual through
treating genomic DNA in the biological sample with bisulfite;
amplifying the bisulfite-treated genomic DNA using primers specific for a CpG site for the one or more markers,
wherein the primers specific for each marker are capable of binding an amplicon bound by the respective primer sequences recited in Table 3, wherein the amplicon bound by the respective primer sequences is at least a portion of a genetic region comprising the respective chromosomal region recited in Table 8A;
determining the methylation level of the CpG site for the one or more markers by methylation-specific PCR, quantitative methylation-specific PCR, methylation-sensitive DNA restriction enzyme analysis, quantitative bisulfite pyrosequencing, or bisulfite genomic sequencing PCR.

90. The method of claim 89, wherein the biological sample is a blood sample, a tissue sample, a fine needle aspirate sample, or a skin tissue sample.

91. The method of claim 89, wherein said CpG site is present in a coding region or a regulatory region.

92. The method of claim 89, wherein said measuring the methylation level of the CpG site for the one or more markers comprises a determination selected from the group consisting of determining the methylation score of said CpG site and determining the methylation frequency of said CpG site.

93. A method for preparing a deoxyribonucleic acid (DNA) fraction from a biological sample of a human individual useful for analyzing one or more genetic loci involved in one or more chromosomal aberrations, comprising:
(a) extracting genomic DNA from a biological sample of a human individual;
(b) producing a fraction of the extracted genomic DNA by:
(i) treating the extracted genomic DNA with bisulfite;
(ii) amplifying the bisulfite-treated genomic DNA using separate primers specific for CpG sites for one or more markers recited in Tables 1A, 2A, 7A, 8A, and 9;
(c) analyzing one or more genetic loci in the produced fraction of the extracted genomic DNA by measuring a methylation level of the CpG site for each of the one or more markers.

94. The method of claim 93, wherein measuring a methylation level of the CpG site for each of the one or more markers is determined by methylation-specific PCR, quantitative methylation-specific PCR, methylation-sensitive DNA restriction enzyme analysis, or bisulfite genomic sequencing PCR.

95. The method of claim 93, wherein amplifying the bisulfite-treated genomic DNA using primers specific for a CpG site for each of the one or more markers is a set of primers that specifically binds at least a portion of a genetic region for the marker as shown in Tables 1A, 2A, 7A, 8A, and/or 9.

96. The method of claim 93, wherein the biological sample is a stool sample, a tissue sample, an organ secretion sample, a CSF sample, a saliva sample, a blood sample, a plasma sample, or a urine sample.

97. The method of claim 93, wherein each of the analyzed one or more genetic loci is associated with melanoma and/or a subtype of melanoma.

98. The method of claim 93, wherein the one or more markers are selected from c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33.

99. The method of claim 93, wherein the one or more markers are selected from AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73.

100. The method of claim 93, wherein the one or more markers are selected from MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763.

101. The method of claim 93, wherein the one or more markers are selected from MAX.chr20.21080958-21081038, MAX.chr7.155259597-155259763, FOXL2NB, and HOXA9_A.

102. A method for preparing a deoxyribonucleic acid (DNA) fraction from a biological sample of a human individual useful for analyzing one or more DNA fragments involved in one or more chromosomal aberrations, comprising:
(a) extracting genomic DNA from a biological sample of a human individual;
(b) producing a fraction of the extracted genomic DNA by:
(i) treating the extracted genomic DNA with bisulfite;
(ii) amplifying the bisulfite-treated genomic DNA using separate primers specific for CpG sites for one or more markers recited in Tables 1A and 2A;
(c) analyzing one or more DNA fragments in the produced fraction of the extracted genomic DNA by measuring a methylation level of the CpG site for each of the one or more markers.

103. The method of claim 102, wherein measuring a methylation level of the CpG site for each of the one or more markers is determined by methylation-specific PCR, quantitative methylation-specific PCR, methylation-sensitive DNA restriction enzyme analysis, or bisulfite genomic sequencing PCR.

104. The method of claim 102, wherein amplifying the bisulfite-treated genomic DNA using primers specific for a CpG site for each of the one or more markers is a set of primers that specifically binds at least a portion of a genetic region for the marker as shown in Tables 1A and/or 2A.

105. The method of claim 102, wherein the biological sample is a stool sample, a tissue sample, an organ secretion sample, a CSF sample, a saliva sample, a blood sample, a plasma sample, or a urine sample.

106. The method of claim 102, wherein each of the analyzed DNA fragments is associated with melanoma and/or a subtype of melanoma.

107. The method of claim 102, wherein the one or more markers are selected from c10orf55, c2orf82, FOXL2NB, CLIC5, FAM174B_B, FLJ22536_A, FOXP1, GALNT3_A, HOPX, HOXA9_A, ITPKA, KCNQ4_A, LMX1B, MAX.chr1.110627096-110627364, MAX.chr10.22541869-22541953, MAX.chr10.62492690-62492812, MAX.chr13.29106812-29106917, MAX.chr17.73073682-73073830, MAX.chr2.71116033-71116122, MAX.chr20.21080958-21081038, MAX.chr5.42992655-42992768, MAX.chr5.60921627-60921853, MAX.chr6.29521537-29521696, MAX.chr7.155259597-155259763, ME3, MIR155HG, NFATC2_A, OCA2, OXT, RNF207_A, RUNX2, SIX4_A, STX16, TAL1, TMEM30B, and TSPAN33.

108. The method of claim 102, wherein the one or more markers are selected from AGRN, BMP8B, c17orf46_B, c17orf57_B, C2CD4A, C2CD4D_B, CDYL, CMAH, DENND2D, DLEU2, DSCR6, FLJ22536_B, FLJ45983, FOXF2, GALNT3_B, HCG4P6, HOXA9_B, KIFC2, LDLRAD2, LY75, LYL1, LYN, MAPK13, MAX.chr1.1072486-1072508, MAX.chr1.32237693-32237785, MAX.chr10.62492374-62492793, MAX.chr11.14926535-14926715, MAX.chr16.54970444-54970469, MAX.chr19.16439332-16439390, MAX.chr20.21080670-21081280, MAX.chr4.113432264-113432298, MAX.chr7.129425668-129425719, MAX.chr8.82543163-82543213, PARP15, PRKAG2, PROC_A, PROM1_B, PTGER4_A, PTP4A3, SDCCAG8, SH3PXD2A, SLC2A2, SLC35D3, TBR1, TBX2, TCP11, TFAP2A, and TRIM73.

109. The method of claim 102, wherein the one or more markers are selected from MAX.chr10.62492680-62492822, TMEM30B_B, MAX.chr11:14926602-14926831, HOXA9_9650, C10orf55_B, MAX.chr17.73073682-73073830, TSPAN33, FAM174B_C, MAX.chr5.60921627-60921853, SIX4_A, KCNQ4_A, FOXP1, C2orf82_B, TAL1_B, BTBD19_B, MAX.chr10.22541874-22541948, ME3, HOPX, MAX.chr20.3229151-3229791, CLIC5, MAX.chr13.29106812-29106917, FOXL2NB, FLJ22536_A, MAX.chr1.110627096-110627364, MAX.chr2.71116033-71116122, MAX.chr20.21080958-21081038, and MAX.chr7.155259597-155259763.

110. The method of claim 102, wherein the one or more markers are selected from MAX.chr20.21080958-21081038, MAX.chr7.155259597-155259763, FOXL2NB, and HOXA9_A.
